(19) United States
(12) Patent Application Publication
(10) Pub. No.: US 2026/0227412 A1
(43) Pub. Date: Aug. 6, 2026

(54) USE OF IFN-I ACTIVITY AS A BIOMARKER FOR TLR INHIBITOR TREATMENT

(71) Applicant: MERCK PATENT GMBH, Darmstadt (DE)

(72) Inventors: Jamie Shaw, Billerica, MA (US); Andrew Bender, Billerica, MA (US); Ankita Deshmukh, Billerica, MA (US); Bharat Vaidyanathan, Billerica, MA (US)

(21) Appl. No.: 19/127,833

(22) PCT Filed: Nov. 8, 2023
(86) PCT No.: PCT/EP2023/081191

§ 371 Date: May 7, 2025

Related U.S. Application Data
(60) Provisional application No. 63/382,964, filed on Nov. 9, 2022.
(60) Provisional application No. 63/495,232, filed on Apr. 10, 2023.
(60) Provisional application No. 63/584,630, filed on Sep. 22, 2023.

(51) Int. Cl. G01N 33/68; A61K 31/4545; A61K 31/4709; A61K 31/5377; A61K 31/573

(52) U.S. Cl. CPC G01N 33/6866; A61K 31/4545; A61K 31/4709; A61K 31/5377; A61K 31/573; G01N 2333/555; G01N 2800/52

(57) ABSTRACT

The present invention provides for the use of IFN-I activity as a predictive biomarker for the treatment of patients with toll-like receptor (TLR) inhibitors and related uses and methods.

### TECHNICAL FIELD OF THE INVENTION

The present invention provides for the use of IFN-I activity as a predictive biomarker for the treatment of patients with toll-like receptor (TLR) inhibitors and related uses and methods.

### BACKGROUND OF THE INVENTION

The TLR family comprises several members with different specificities and is part of the cellular pathogen pattern recognition system, which has evolved as a defense against a variety of infections. The functional expression of selected TLRs in tissues is highly different. Some receptors are located at the cell surface such as TLR4 (stimulated by E. coli lipopolysaccharide LPS), e.g., on epithelial cells, whereas others, such as TLR3, 7, 8 and 9, are located at endosomal membranes of specific immune cells. The latter are all activated by nucleic acids but recognize various types of them. For instance, TLR9 is activated by single stranded DNA containing CpG subsequences, TLR7 and 8 are activated by single stranded RNA, and TLR3 is activated by double-stranded RNA. The activation of the TLRs triggers various downstream signaling cascades, including the signaling via nuclear factor -kB (NF-kB), interferon (IFN) response factors (IRFs), and mitogen-activated protein (MAP) kinases, to result in the transcription of various immune response genes, including inflammatory cytokines, stimulatory immune cytokines, chemokines and co-stimulatory molecules (Farrugia and Baron, Int J Inflam. 2017; 2017:8391230).

TLRs have been implicated in various autoimmune and inflammatory diseases, with the clearest example being the role played by TLR7 in the pathogenesis of systemic lupus erythematosus (Barrat and Coffman, Immunol Rev, 223:271-283, 2008). TLR7 has also been implicated in systemic sclerosis, myositis and rheumatoid arthritis (Duffy and O'Reilly, Immunotargets Ther. 2016; 5:69-80). TLR8 in turn has inter alia been associated with rheumatoid arthritis and systemic sclerosis (Duffy and O'Reilly, loc. cit).

Type I interferons (IFN-I) are also part of the innate immune response against pathogens. They form part of one of several parallel signaling cascades that are triggered, e.g., by TLR7 and TLR8. Apart from TLRs, IFN-I can be induced by an array of other host pattern recognition receptors following the recognition of pathogen components, including Rig-I-like receptors (RLRs), NOD-like receptors (NLRs) and DNA sensors. The released IFN-I then binds to the IFN-α receptor (IFNAR), triggering a signaling cascade that leads to the expression of interferon-stimulated genes (ISGs). Just like TLRs, IFN-I signaling has also been implicated in various diseases, in particular, autoimmune diseases and IFN-I activity has been explored as a possible biomarker for such diseases by measuring the expression of one or more genes that are modulated by IFN-I. For some treatments, a correlation between clinical response to the treatment and the IFN-I activity has been observed, albeit in some cases a positive correlation was observed whereas in other cases a negative correlation was observed (Psarras et al., Rheumatology (Oxford), 2017 Oct. 1;56 (10): 1662-1675).

There remains a need to identify patients that are more likely to respond to treatment with TLR inhibitors.

### SUMMARY OF THE INVENTION

The present invention relates to the use of IFN-I activity as a predictive biomarker for treatment outcome to therapy with TLR inhibitors.

In one aspect, the invention provides a method for predicting therapeutic effectiveness of a TLR inhibitor in an individual with a disease comprising determining the IFN-I activity in a sample from the individual, wherein the IFN-I activity in the sample indicates the therapeutic effectiveness of the TLR inhibitor.

In another aspect, the invention provides a method for predicting the suitability of an individual with a disease to initiate treatment with a TLR inhibitor comprising determining the IFN-I activity in a sample from the individual, wherein the IFN-I activity in the sample indicates the suitability of the individual to initiate the treatment.

In another aspect, the invention provides a method for predicting the suitability of an individual with a disease who is receiving treatment with a TLR inhibitor to continue the treatment comprising determining the IFN-I activity in a sample from the individual, wherein the IFN-I activity in the sample indicates the suitability of the individual to continue the treatment.

In another aspect, the invention provides a TLR inhibitor for use in a method of treating a disease in an individual comprising administering the TLR inhibitor to the individual, wherein treatment is based upon IFN-I activity in a sample from the individual.

IFN-I activity may either be assessed directly or indirectly, e.g., by determining the expression of an IFN-I signature of an individual. Such IFN-I signature may comprise one or more genes selected from the group consisting of BST2, CMPK2, CXCL10, EPSTII, GBP5, HERC5, HERC6, IFI6, IFI27, IFI44, IFI44L, IFIH1, IFIT1, IFIT2, IFIT3, IRF7, ISG15, LY6E, MX1, MX2, OAS1, OAS2, OAS3, OASL, PKR, RSAD2, SIGLECI, STATI, TNFSF10 and USP18. For instance, it may comprise HERC5, IFI27, IFIT1 and RSAD2.

In some embodiments, the TLR inhibitor is a TLR7 and/or TLR8 inhibitor. For instance, the TLR7 and/or TLR8 inhibitor could be selected from the group consisting of 5-[(3R,5S)-3-amino-5-(trifluoromethyl) piperidin-1-yl]quinoline-8-carbonitrile; (3R,5S)-1-(8-methoxy-1,7-naphthyridin-5-yl)-5-methylpiperidin-3-amine; 2-{4-[2-(7,8-dimethyl[1,2,4]triazolo[1,5-a]pyridin-6-yl)-3-(propan-2-yl)-1H-indol-5-yl]piperidin-1-yl}acetamide; rel-(2R,6R)-4-(8-cyanoquinolin-5yl)-N-((3R,4S)-4-fluoropyrrolidin-3-yl)-6-methylmorpholine-2-carboxamide hydrochloride; (S)—N-(4-((5-(1,6-dimethyl-1H-pyrazolo[3,4-b] pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide; and (R)—N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide or a pharmaceutically acceptable salt of any of these compounds.

### BRIEF DESCRIPTION OF THE DRAWINGS

FIG. 1 shows a) the correlation, as indicated by the correlation coefficient r, between the IFN-I signature scores of various IFN-I signatures as determined for a population of SLE patients and b) an exemplary graph plotting the IFN-I signature scores of the Dx_4 IFN-I signature against the IFN-I signature scores of the EMD_9 IFN-I signature as determined for said SLE patient population.

FIG. 2 shows the cumulative distribution function of a) time to recovery and b) time to clinical deterioration in unstratified patient populations receiving either placebo or 50 mg or 100 mg enpatoran BID (safety analysis set).

FIG. 3 shows the cumulative distribution function of time to recovery for patients with a) high and b) low IFN-I signature scores at baseline and receiving either placebo or 50 mg or 100 mg enpatoran BID.

FIG. 4 shows that CMPD2 reverses the ability of IFN-α pre-treatment to reduce Dex potency. (A) Dose response curve of Dex impacting IL-6 in PBMCs with/without IFN-α pretreatment at 16 hours post-R848 stimulation. (B)Representative bar graph of IL-6 secretion following treatment with 41 nM Dex and/or 7.8 nM CMPD2 in the presence of absence of IFN-α pre-treatment. (C) Quantification of synergy scores from Combenefit Loewe matrix plots (area under the curve) of Dex and CMPD2 interactions for IL-6 inhibition in R848-stimulated PBMCs either untreated or pre-treated with IFNa at 16 hours post-stimulation. Figures (A-C) show pooled data from 5 donors. In (A) and (B), the data are normalized to the R848 stimulation control. Paired t-test: ** p<0.005.

FIG. 5 shows that CMPD2 reverses the ability of IFN-α pre-treatment to reduce Dex potency. Dex dose response curves impacting IL-6 either alone or with different doses of CMPD2 at 16 hours post-R848 stimulation in the absence (A) or presence (B) of IFN-α pre-treatment. Combenefit analysis showing Loewe matrix plots of Dex and CMPD2 interactions for IL-6 inhibition in R848 stimulated cells at 16 hours post-stimulation in the absence (B) or presence (D) of IFNa pre treatment. For (A) and (C), the data are normalized to the R848 control and pooled from 5 donors.

FIG. 6 shows TLR7/8 activation in human PBMCs by patient-derived immune complexes. IgG was isolated from plasma samples from HC and patients with SLE, LN, IBM, PM and DM and combined with necrotic cell lysate to form immune complexes that were used to stimulate healthy donor PBMCs. A) After 24 hours of treatment, supernatants were collected from the PBMCs and IFN-α was measured by AlphaLISA. Means from 2-4 experiments for each IgG are shown and each symbol represents an IgG sample. B) For samples that had stimulating activity, the PBMCs were pre-treated with enpatoran for 30 minutes before the immune complex was added to the cells and IFN-α was measured following 24 hours of treatment. All IgG samples were tested with 2-4 healthy donor PBMCs. DM dermatomyositis, HC healthy control, IBM inclusion body myositis, IC immune complex, IFN-α interferon-alpha, IgG immunoglobulin G, PBMCs peripheral blood mononuclear cells, PM polymyositis, SLE systemic lupus erythematosus, TLR7/8 toll-like receptor 7/8.

FIG. 7 shows gene expression changes induced by patient-derived immune complexes. IgG was isolated from plasma samples from HC and patients with SLE, LN, IBM, PM and DM and then combined with necrotic cell lysate to form immune complexes that were used to stimulate healthy donor PBMCs. After 24 hours of treatment, the cells were collected and analyzed by NanoString to measure changes in gene expression. A) Heat map shows the Log2 FC compared to HC IgG samples. Each column represents a separate IgG sample, and the patient group is indicated by shading. The samples that stimulated IFN-α protein production are indicated by black the black bar above the columns. B) An IFN-I signature score was calculated using the ISGs indicated in the heat map and scores were plotted for each individual sample. Data are averaged from independent experiments run with 2 PBMC donors for each IgG sample. DM dermatomyositis, FC fold change, HC healthy control, IBM inclusion body myositis, IFN-α interferon-alpha, IgG immunoglobulin G, ISGs interferon-stimulated genes, PBMCs peripheral blood mononuclear cells, PM polymyositis, SLE systemic lupus erythematosus.

### DETAILED DESCRIPTION

Each of the embodiments described herein can be combined with any other embodiment described herein not inconsistent with the embodiment with which it is combined. Furthermore, unless incompatible in a given context, wherever a compound is stipulated which is capable of ionization (e.g., protonation or deprotonation), the definition of said compound includes any pharmaceutically acceptable salts thereof. Accordingly, the phrase “or a pharmaceutically acceptable salt thereof” is implicit in the description of all compounds described herein.

The present invention may be understood more readily by reference to the detailed description above and below of the particular and preferred embodiments of the invention and the examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art. So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

### General Definitions

“A”, “an”, and “the” include plural referents unless the context clearly dictates otherwise. As such, the terms “a” (or “an”), “one or more”, and “at least one” are used interchangeably herein.

The term “about” when used to modify a numerically defined parameter refers to any minimal alteration in such parameter that does not change the overall effect, e.g., the efficacy of the agent in treatment of a disease or disorder. In some embodiments, the term “about” means that the parameter may vary by as much as 10% below or above the stated numerical value for that parameter.

The term “around” when used in relationship to a value, e.g., the upper limit of a range or a reference value, such as the reference IFN-I activity or the reference IFN-I signature score, refers to any value that is reasonably close to the value referred to. For instance, any value above the 90th or 95th percentile. In some embodiments, the term “around” means the exact value.

“Administering” or “administration of” a drug to a patient (and grammatical equivalents of this phrase) refers to direct administration, which may be administration to a patient by a medical professional or may be self-administration, and/or indirect administration, which may be the act of prescribing a drug, e.g., a physician who instructs a patient to self-administer a drug or provides a patient with a prescription for a drug is administering the drug to the patient. It is understood that the therapeutic agents mentioned herein, such as the TLR inhibitor, are administered in a therapeutically effective amount.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy, e.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

“Combination treatment” or “in combination with” as used herein denotes any form of concurrent, parallel, simultaneous, sequential or intermittent treatment with at least two distinct treatment modalities (i.e., compounds, components, targeted agents, therapeutic agents or therapies). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or dosage forms) or separately (e.g., on the same day or on different days and in any order as according to an appropriate dosing protocol for the separate compositions, formulations or dosage forms) in a manner and dosing regimen prescribed by a medical care taker or according to a regulatory agency. In general, each treatment modality will be administered at a dose and/or on a time schedule determined for that treatment modality. Optionally, four or more modalities may be used in a combination treatment. Additionally, the combination treatments provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“IFN-I activity” refers to the level of activity of the type I interferons. In some embodiments, the IFN-I activity refers to the level of signaling activity of the type I interferons as reflected, e.g., by the level of expression of ISGs.

An “IFN-I signature” refers to one or more genes whose expression is modulated by IFN-I and whose expression pattern is reflective of IFN-I activity.

An “IFN-I signature score” refers to the arithmetic mean of the normalized expression levels of the genes in an IFN-I signature.

An “IFN-I signature expression pattern” refers to the expression levels of the genes in an IFN-I signature.

“Patient”, “subject” and “individual” are used interchangeably herein to refer to a mammal in need of treatment for a disease or disorder. Generally, the “patient”, “subject” or “individual” is a human diagnosed or at risk for suffering from one or more symptoms of a disease or disorder. In certain embodiments a “patient”, “subject” or “individual” may refer to a non-human mammal, such as a non-human primate, a dog, cat, rabbit, pig, mouse, or rat, or animals used, e.g., in screening, characterizing, and evaluating drugs and therapies.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

A “prediction” as referred to herein of, e.g., the therapeutic effectiveness of a TLR inhibitor, the suitability of an individual with a disease to initiate treatment with a TLR inhibitor or the suitability of an individual with a disease who is receiving treatment with a TLR inhibitor to continue the treatment only provides an indication about the likelihood of a treatment outcome with a TLR inhibitor but does not predict the treatment outcome with certainty. For instance, an individual for which the TLR inhibitor is predicted to be therapeutically effective just has a higher likelihood of therapeutic effectiveness, whereas an individual for which the TLR inhibitor is predicted not to be therapeutically effective just has a lower likelihood of therapeutic effectiveness. Similarly, an individual that is predicted to be suitable to initiate treatment with a TLR inhibitor just has a higher likelihood that the treatment in such individual with a TLR inhibitor will be therapeutically effective, whereas an individual that is predicted not to be suitable to initiate treatment with a TLR inhibitor just has a lower likelihood that the treatment in such individual with a TLR inhibitor will be therapeutically effective. Similarly, an individual that is predicted to be suitable to continue treatment with a TLR inhibitor just has a higher likelihood that the treatment in such individual with a TLR inhibitor will be therapeutically effective, whereas an individual that is predicted not to be suitable to continue treatment with a TLR inhibitor just has a lower likelihood that the treatment in such individual with a TLR inhibitor will be therapeutically effective.

A “reference IFN-I activity”, “reference IFN-I signature expression pattern” or “reference IFN-I signature score” allows to identify patients that are more likely to respond to treatment with a TLR inhibitor and/or patients that are less likely to respond to treatment with a TLR inhibitor, e.g., based on a comparison of the reference IFN-I activity, reference IFN-I signature expression pattern or reference IFN-I signature score with the IFN-I activity, IFN-I signature expression pattern or IFN-I signature score of such patients, respectively. Which IFN-I activities, IFN-I signature expression patterns or IFN-I signature scores indicate a which treatment outcome depends on how the corresponding reference was defined. For instance, the reference IFN-I activity or reference IFN-I signature score could be defined to divide the patients between those that are more likely to respond to treatment with a TLR inhibitor from those that are less likely to respond. In such case, those patients having an IFN-I activity above the reference IFN-I activity or an IFN-I signature score above the reference IFN-I signature score are more likely to respond to treatment with a TLR inhibitor than those patients having an IFN-I activity below the reference IFN-I activity or an IFN-I signature score below the reference IFN-I signature score. Similarly, a reference IFN-I activity or reference IFN-I signature score that is characteristic of the patient population that is more likely to respond to the TLR inhibitor treatment, e.g., the arithmetic mean or median of the IFN-I activity or IFN-I signature score in this patient population, may be defined and a patient having an IFN-I activity or IFN-I signature score around or above the respective reference is determined to be more likely to respond to the treatment (and vice versa). Similarly, for instance, a patient having an expression of the genes of the IFN-I signature similar to the expression pattern of these genes that is characteristic of the patient population that is more likely to respond to treatment with a TLR inhibitor, i.e. a reference IFN-I signature expression pattern, is likewise more likely to respond to the TLR treatment and vice versa. It is understood that the IFN-I signature of the reference IFN-I signature expression pattern or the IFN-I signature on which the reference IFN-I signature score is based is identical to the IFN-I signature that is used for determining the IFN-I signature expression pattern or the IFN-I signature score of the patient whose treatment outcome shall be predicted according to the methods or uses of the invention. The person skilled in the art is well aware about how to define such reference IFN-I activity, reference IFN-I signature expression pattern or reference IFN-I signature score. For instance, it may be derived from a post-hoc analysis of a patient population, wherein the IFN-I activity, IFN-I signature expression pattern or IFN-I signature score of the different patients before treatment is compared to the treatment outcome of the different patients to then divide the population into patients where treatment was more effective and patients where treatment was less effective and, e.g., define a threshold value for the IFN-I activity or IFN-I signature score that divides these patient populations or define an IFN-I activity, IFN-I signature score or IFN-I signature expression pattern for either or both of these patient populations that is characteristic for these. Such defined IFN-I activity, IFN-I signature expression pattern or IFN-I signature score may then serve as as the reference IFN-I activity, reference IFN-I signature expression pattern and reference IFN-I signature score, respectively. Similarly, the reference IFN-I activity, reference IFN-I signature expression pattern or reference IFN-I signature score may be derived, e.g., from a healthy population, as such population may be regarded as a baseline or as a population having low IFN-I activity, since the IFN-I pathway is generally not very active in a healthy individual. Accordingly, the IFN-I activity, IFN-I signature expression pattern or IFN-I signature score of the different individuals in such healthy population can be determined and the IFN-I activity or IFN-I signature score that is, e.g., around the upper limit of the determined ranges or corresponding to the arithmetic mean or median may then be defined as the reference IFN-I activity or reference IFN-I signature score, respectively. Similarly, the expression pattern of the genes in the IFN-I signature that is characteristic for such population may be defined as the reference IFN-I signature expression pattern. In some embodiments, having “high” IFN-I activity means an IFN-I activity above a reference IFN-I activity, e.g., as reflected by an IFN-I signature score above a reference IFN-I signature score, and having “low” IFN-I activity means an IFN-I activity below a reference IFN-I activity, e.g., as reflected by an IFN-I signature score below a reference IFN-I signature score.

A “sample” as referred to herein is any biological sample from an individual that allows the determination of IFN-I activity, e.g., the determination of an IFN-I signature expression pattern or IFN-I signature score. The sample may, for instance, refer to fluids, cells and tissues of an individual. It may refer to a blood sample and extracted RNA of an individual.

A “small molecule” is a chemical, usually organic, compound with low molecular weight, such as ≤1000 daltons or ≤900 daltons.

“Therapeutically effective amount” of a therapeutic agent refers to an amount effective, at dosages and for periods of time necessary, that, when administered to a patient, will have the intended therapeutic effect, e.g., alleviation, amelioration, palliation, or elimination of one or more manifestations of the disease or disorder in the patient, or any other clinical result in the course of treating the patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of a therapeutic agent to elicit a desired response in the individual. A therapeutically effective amount is also one in which any toxic or detrimental effects of a therapeutic agent are outweighed by the therapeutically beneficial effects.

“Therapeutic effectiveness” refers to a favorable treatment response, e.g., alleviation, amelioration of one or more symptoms of a disease; diminishment of extent of disease; delay or slowing of disease progression; amelioration, palliation, or stabilization of the disease state; or other beneficial results. For instance, therapeutic effectiveness may refer to the time to recovery from a certain disease, e.g., COVID-19.

“TLR inhibitor” refers to a compound that inhibits the activity of one or more members of the human TLR family of proteins through direct interaction between the TLR inhibitor and the TLR. The TLR inhibitor may function, for instance, by stabilizing the TLR in its resting, inactive state. In some embodiments, the TLR inhibitor inhibits the activity of human TLR7 (also referred to as a “TLR7 inhibitor”). In some embodiments, the TLR inhibitor inhibits the activity of human TLR8 (also referred to as a “TLR8 inhibitor”). In some embodiments, the TLR inhibitor inhibits the activity of human TLR7 and/or TLR8 (also referred to as a “TLR7 and/or TLR8 inhibitor”). In some embodiments, the TLR inhibitor inhibits the activity of human TLR7 and TLR8 (also referred to as a “TLR7 and TLR8 inhibitor”). In some embodiments, the TLR inhibitor selectively inhibits human TLR7 and/or TLR8. The TLR inhibitor may, for example, be a small molecule, a nucleic acid, such as an oligonucleotide, or a polypeptide, such as an antibody. In some embodiments, the TLR inhibitor is a small molecule. Possible effects of the inhibition of the TLR pathway include the suppression of inflammatory processes. Inhibition in this context need not be complete or 100%. Instead, inhibition means reducing, decreasing or abrogating the activity of the TLR pathway or inflammatory processes, respectively. Inhibition may be 10%, 20%, 30%, 40%, 50%, 60%, 70%, 80%, 90%, 95% or 100%, or a statistically significant inhibition when compared to a control. In some embodiments, the IC50 value of the TLR inhibitor for the inhibition of a TLR is below 10 μM, below 2 μM, below 1 μM, below 250 nM, below 100 nM, below 50 nM or below 25 nM. In some embodiments, the IC50 value is determined in HEK293 cells. In an exemplary protocol, HEK293 cells are stably transfected with either TLR7 or TLR8 and an NF-kappaB-luciferase reporter gene. For testing the TLR inhibitor, cells are seeded in 384-well black, clear bottom plates and after an overnight incubation at 37° C. and 5% CO2, TLR inhibtor dilutions are added in duplicate. The cells are then stimulated with 10 μM R848 or 30 μM R848 for testing in HEK TLR7 or HEK TLR8 cells respectively. Following incubation for 5 hours at 37° C. and 5% CO2, SteadyGlo substrate reagent (Promega, Madison, WI) is added to each well and luminescence is measured, e.g., using the Perkin Elmer Envision Multilabel Reader.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a disease; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, a plurality of items, structural elements, compositional elements, and/or materials may be presented in a common list for convenience. However, these lists should be construed as though each member of the list is individually identified as a separate and unique member.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Chemical Compound Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Exemplary aliphatic groups are linear or branched, substituted or unsubstituted C₁-C₈ alkyl, C₂-C₅ alkenyl, C₂-C₅ alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, or phosphorus (including, any oxidized form of nitrogen, sulfur, or phosphorus; the quaternized form of any basic nitrogen or; a substitutable nitrogen of a heterocyclic ring, for example N(as in 3,4-dihydro-2H-pyrrolyl), NH(as in pyrrolidinyl)_{0r NR}⁺ (as in N-substituted pyrrolidinyl)).

The term “unsaturated”, as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” is used interchangeably with the term “aryl ring”. In certain embodiments of the present invention, “aryl” refers to an aromatic ring system. Exemplary aryl groups are phenyl, biphenyl, naphthyl, anthracyl and the like, which optionally includes one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group is optionally mono- or bicyclic. The term “heteroaryl” is used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen is N(as in 3,4-dihydro-2H-pyrrolyl), NH(as in pyrrolidinyl), or +NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group is optionally mono-or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, certain compounds contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

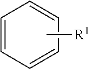

refers to at least

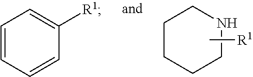

refers to at least

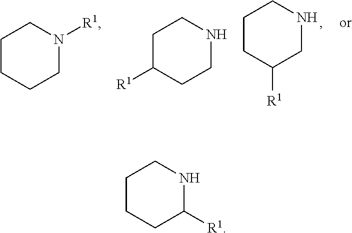

Unless otherwise indicated, an “optionally substituted” group has a suitable substituent at each substitutable position of the group, and when more than one position in any given structure is substituted with more than one substituent selected from a specified group, the substituent is either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently deuterium; halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which are optionally substituted with R^{∘}; —(CH₂)₀₋₄O(CH₂)₀₋₁Ph which is optionally substituted with R^{∘}; —CH—CHPh, which is optionally substituted with R^{∘}; —(CH₂)₀₋₄O(CH₂)₀₋₁-pyridyl which is optionally substituted with R^{∘}; —NO₂; —CN; —N₃; —(CH₂)₀₋₄N(R^{∘}₂; —(CH₂)₀₋₄N(R^{∘}C(O)R^{∘}; —N(R^{∘}C(S)R^{∘}; —(CH₂)₀₋₄N(R^{∘}C(O)NR^{∘}₂; —N(R^{∘}C(S)NR^{∘}₂; —(CH₂)₀₋₄N(R^{∘}C(O)OR^{∘}; —N(R^{∘}N(R^{∘}C(O)R^{∘}; —N(R^{∘}N(R^{∘}C(O)NR^{∘}₂; —N(R^{∘}N(R^{∘}C(O)OR^{∘}; —(CH₂)₀₋₄C(O)R^{∘}; C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}2; —C(S)NR^{∘}₂; —C(S)SR^{∘}; —SC(S) SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘}S(O)₂NR^{∘}₂; —N(R^{∘}S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})hd2; SiR^{∘}₃; —(C₁₋₄ straight or branched alkylene)O—N(R^{∘})₂; or —(C₁₋₄ straight or branched) alkylene) C(O)O—N(R^{∘})₂, wherein each R^{∘}is optionally substituted as defined below and is independently hydrogen, C₁₋₆ aliphatic, CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono-or bicyclic ring having 0-4 heteroatoms independently selected from nitrogen, oxygen, or sulfur, which is optionally substituted as defined below.

Suitable monovalent substituents on R^{∘}(or the ring formed by taking two independent occurrences of R^{∘}together with their intervening atoms), are independently deuterium, halogen, —(CH₂)₀₋₂R^{•}, -(haloR^{•}, —(CH₂)₀₋₂OH, —(CH₂)₀₋₂OR^{•}, —(CH₂)₀₋₂CH(OR^{•}₂; —O(haloR^{•}, —CN, —N₃, (CH₂)₀₋₂C(O)R^{•}, (CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O) SR′, —(C₁₋₄ straight or branched alkylene) C(O)OR′, or —SSR′ wherein each R^{∘}is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘}include ═O and ═S.

Suitable divalent substituents on a saturated carbon atom of an “optionally substituted” group include the following: ═O, —S, —NNR*₂, ═NNHC(O)R*, ═NNHC(O)OR*, ═NNHS(O)₂R*, ═NR*, ═NOR*, —O(C(R*₂))₂-3O—, or —S(C(R*₂))₂₋₃S—, wherein each independent occurrence of R* is selected from hydrogen, C₁₋₆ aliphatic which is substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which is optionally substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}, —OH, —OR^{•}, —O(haloR^{•}, —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{t}, —NR^{t}₂, —C(O)R^{t}, —C(O)OR^{t}, —C(O) C(O)R^{t}, —C(O)CH₂C(O)R^{•}, —S(O)₂R^{t}, —S(O)₂NR+₂, C(S)NR₁₂, C(NH)NR+₂, or —N(R^{t})S(O)₂R^{t}; wherein each R^{t} is independently hydrogen, C₁₋₆ aliphatic which is optionally substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{T}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{t} are independently halogen, —R^{•}, -(haloR^{•}, —OH, OR)—O (haloR^{•}, —CN, —C(O)OH, —C(O)OR′, —NH₂, —NHR′, —NR^{∘}2, or —NO₂, wherein each R^{∘}is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

In certain embodiments, the terms “optionally substituted”, “optionally substituted alkyl,” “optionally substituted “optionally substituted alkenyl,” “optionally substituted alkynyl”, “optionally substituted carbocyclic,” “optionally substituted aryl”, “optionally substituted heteroaryl,” “optionally substituted heterocyclic,” and any other optionally substituted group as used herein, refer to groups that are substituted or unsubstituted by independent replacement of one, two, or three or more of the hydrogen atoms thereon with typical substituents including, but not limited to:
—F, —C₁, —Br, -I, deuterium,
—OH, protected hydroxy, alkoxy, oxo, thiooxo,
—NO₂, —CN, CF₃, N₃,
—NH₂, protected amino, —NH alkyl, —NH alkenyl, —NH alkynyl, —NH cycloalkyl, —NH-aryl, —NH-heteroaryl, —NH-heterocyclic, -dialkylamino, -diarylamino, -diheteroarylamino,
—O-alkyl, —O-alkenyl, —O-alkynyl, —O-cycloalkyl, —O-aryl, —O-heteroaryl, —O-heterocyclic, —C(O)-alkyl, —C(O)-alkenyl, —C(O)-alkynyl, —C(O)-carbocyclyl, —C(O)-aryl, —C(O)-heteroaryl, —C(O)-heterocyclyl,
CONH₂, —CONH-alkyl, —CONH-alkenyl, —CONH-alkynyl, —CONH-carbocyclyl, -CONH-aryl, —CONH-heteroaryl, —CONH-heterocyclyl, —OCO₂-alkyl, —OCO₂-alkenyl, —OCO₂-alkynyl, —OCO₂-carbocyclyl, —OCO₂-aryl, —OCO₂-heteroaryl, —OCO₂-heterocyclyl, —OCONH2, —OCONH-alkyl, —OCONH-alkenyl, —OCONH-alkynyl, —OCONH-carbocyclyl, —OCONH-aryl, —OCONH-heteroaryl, —OCONH-heterocyclyl, —NHC(O)-alkyl, —NHC(O)-alkenyl, —NHC(O)-alkynyl, —NHC(O)-carbocyclyl, —NHC(O)-aryl, —NHC(O)-heteroaryl, —NHC(O)-heterocyclyl, —NHCO2-alkyl, —NHCO2-alkenyl, —NHCO₂-alkynyl, —NHCO₂-carbocyclyl, —NHCO₂-aryl, —NHCO₂-heteroaryl, —NHCO2-heterocyclyl, —NHC(O)NH₂, —NHC(O)NH-alkyl, —NHC(O)NH-alkenyl, —NHC(O)NH-alkenyl, —NHC(O)NH-carbocyclyl, —NHC(O)NH-aryl, —NHC(O)NH-heteroaryl, —NHC(O)NH-heterocyclyl, NHC(S)NH₂, —NHC(S)NH-alkyl, —NHC(S)NH-alkenyl, —NHC(S)NH-alkynyl, —NHC(S)NH-carbocyclyl, —NHC(S)NH-aryl, —NHC(S)NH-heteroaryl, —NHC(S)NH-heterocyclyl, —NHC(NH)NH₂, —NHC(NH)NH—alkyl, —NHC(NH)NH--alkenyl, —NHC(NH)NH— alkenyl, —NHC(NH)NH-carbocyclyl, —NHC(NH)NH-aryl, —NHC(NH)NH-heteroaryl, —NHC(NH)NH-heterocyclyl, —NHC(NH)-alkyl, —NHC(NH)-alkenyl, —NHC(NH)-alkenyl, —NHC(NH)-carbocyclyl, —NHC(NH)-aryl, —NHC(NH)-heteroaryl, —NHC(NH)-heterocyclyl, —C(NH)NH-alkyl, —C(NH)NH-alkenyl, —C(NH)NH-alkynyl, —C(NH)NH-carbocyclyl, —C(NH)NH-aryl, —C(NH)NH-heteroaryl, —C(NH)NH-heterocyclyl, —S(O)-alkyl, —S(O)-alkenyl, —S(O)-alkynyl, —S(O)-carbocyclyl, —S(O)-aryl, —S(O)-heteroaryl, —S(O)-heterocyclyl, —SO₂NH₂, —SO₂NH-alkyl, —SO₂NH-alkenyl, —SO₂NH-alkynyl, —SO₂NH-carbocyclyl, —SO₂NH-aryl, —SO₂NH-heteroaryl, —SO₂NH-heterocyclyl, —NHSO₂-alkyl, —NHSO₂-alkenyl, —NHSO₂-alkynyl, —NHSO₂-carbocyclyl, —NHSO₂-aryl, —NHSO₂-heteroaryl, —NHSO₂-heterocyclyl,
—CH₂NH₂, —CH₂SO₂CH₃,
mono-, di-, or tri-alkyl silyl,
alkyl, -alkenyl, -alkynyl, -aryl, -arylalkyl, -heteroaryl, -heteroarylalkyl, -heterocycloalkyl,
cycloalkyl, -carbocyclic, -heterocyclic, polyalkoxyalkyl, polyalkoxy, -methoxymethoxy, -methoxyethoxy, —SH, —S-alkyl, —S-alkenyl, —S-alkynyl, —S-carbocyclyl, —S-aryl, —S-heteroaryl, -S-heterocyclyl, or methylthiomethyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted and compounds referred to herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds described herein are within the scope of the invention.

Additionally, unless otherwise stated, structures depicted and compounds referred to herein are also meant to include compounds that differ only in the presence of one or more isotopically enriched atoms. For example, compounds having the present structures including the replacement of hydrogen by deuterium or tritium, or the replacement of a carbon by a ¹³C-or ¹⁴C-enriched carbon are within the scope of this invention. In some embodiments, the group comprises one or more deuterium atoms.

There is furthermore intended that, unless otherwise stated, compounds referred to herein include isotope-labeled forms thereof. An isotope-labeled form of a compound referred to herein is identical to this compound apart from the fact that one or more atoms of the compound have been replaced by an atom or atoms having an atomic mass or mass number which differs from the atomic mass or mass number of the atom which usually occurs naturally. Examples of isotopes which are readily commercially available and which can be incorporated into a compound by well-known methods include isotopes of hydrogen, carbon, nitrogen, oxygen, phosphorus, fluo-rine and chlorine, for example ²H, ³H, ¹³C, ¹⁴C, ¹⁵N, ¹⁸O, ¹⁷O, ³¹P, ³²P, ³⁵S, ¹⁸F and ³⁶CI, respectively. A compound referred to herein, a prodrug, thereof or a pharmaceutically acceptable salt of either which contains one or more of the above-mentioned isotopes and/or other isotopes of other atoms is intended to be part of the present invention. An isotope-labeled compound can be used in a number of beneficial ways. For example, an isotope-labeled compound into which, for example, a radioisotope, such as 3H or ¹⁴C, has been incorporated, is suitable for medicament and/or substrate tissue distribution assays. These radioisotopes, i.e. tritium (3H) and carbon-14 (¹⁴° C.), are particularly preferred owing to simple preparation and excellent detectability. Incorporation of heavier isotopes, for example deuterium (2H), into a compound has therapeutic advantages owing to the higher metabolic stability of this isotope-labeled compound. Higher metabolic stability translates directly into an increased in vivo half-life or lower dosages, which under most circumstances would represent a preferred embodiment of the present invention. An isotope-labeled compound can usually be prepared by carrying out the procedures disclosed in the synthesis schemes and the related description, in the example part and in the preparation part in the present text, replacing a non-isotope-labeled reactant by a readily available isotope-labeled reactant.

Deuterium (H) can also be incorporated into a compound referred to herein for the purpose in order to manipulate the oxidative metabolism of the compound by way of the primary kinetic isotope effect. The primary kinetic isotope effect is a change of the rate for a chemical reaction that results from exchange of isotopic nuclei, which in turn is caused by the change in ground state energies necessary for covalent bond formation after this isotopic exchange. Exchange of a heavier isotope usually results in a lowering of the ground state energy for a chemical bond and thus causes a reduction in the rate in rate-limiting bond breakage. If the bond breakage occurs in or in the vicinity of a saddle-point region along the coordinate of a multi-product reaction, the product distribution ratios can be altered substantially. For explanation: if deuterium is bonded to a carbon atom at a non-exchangeable position, rate differences of k_{M}/k_{D}=2-7 are typical. If this rate difference is successfully applied to a compound that is susceptible to oxidation, the profile of this compound in vivo can be drastically modified and result in improved pharmacokinetic properties.

When discovering and developing therapeutic agents, the person skilled in the art is able to optimize pharmacokinetic parameters while retaining desirable in vitro properties. It is reasonable to assume that many compounds with poor pharmacokinetic profiles are susceptible to oxidative metabolism. In vitro liver microsomal assays currently available provide valuable information on the course of oxidative metabolism of this type, which in turn permits the rational design of deuterated compounds with improved stability through resistance to such oxidative metabolism. Significant improvements in the pharmacokinetic profiles of compounds are thereby obtained, and can be expressed quantitatively in terms of increases in the in vivo half-life (t/2), concentration at maximum therapeutic effect (Cₘₐₓ), area under the dose response curve (AUC), and F; and in terms of reduced clearance, dose and materials costs.

The following is intended to illustrate the above: a compound which has multiple potential sites of attack for oxidative metabolism, for example benzylic hydrogen atoms and hydrogen atoms bonded to a nitrogen atom, is prepared as a series of analogues in which various combinations of hydrogen atoms are replaced by deuterium atoms, so that some, most or all of these hydrogen atoms have been replaced by deuterium atoms. Half-life determinations enable favorable and accurate determination of the extent of the extent to which the improvement in resistance to oxidative metabolism has improved. In this way, it is determined that the half-life of the parent compound can be extended by up to 100% as the result of deuterium-hydrogen exchange of this type.

Deuterium-hydrogen exchange in a compound can also be used to achieve a favorable modification of the metabolite spectrum of the starting compound in order to diminish or eliminate undesired toxic metabolites. For example, if a toxic metabolite arises through oxidative carbon-hydrogen (C—H) bond cleavage, it can reasonably be assumed that the deuterated analogue will greatly diminish or eliminate production of the unwanted metabolite, even if the particular oxidation is not a rate-determining step. Further information on the state of the art with respect to deuterium-hydrogen exchange may be found, for example in Hanzlik et al., J. Org. Chem. 55, 3992-3997, 1990, Reider et al., J. Org. Chem. 52, 3326-3334, 1987, Foster, Adv. Drug Res. 14, 1-40, 1985, Gillette et al, Biochemistry 33 (10) 2927-2937, 1994, and Jarman et al. Carcinogenesis 16 (4), 683-688, 1993.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### Methods of Use

The present invention relates to the use of IFN-I activity as a predictive biomarker for the treatment of an individual suffering from a disease or disorder with a TLR inhibitor. In particular, it has been found that a TLR inhibitor is more likely to be therapeutically effective in an individual having high IFN-I activity. The IFN-I activity may be determined, e.g., indirectly by measuring the IFN-I expression level with a PBMC assay as outlined in Example 4 or with an IFN-I signature that is reflective of IFN-I activity. Such IFN-I signature may then serve to determine an IFN-I signature score or IFN-I signature expression pattern and through comparison to a reference IFN-I signature score or reference IFN-I signature expression pattern, respectively, it can be determined whether the individual has high or low IFN-I activity. Such biomarker information may be used, for instance, to predict the suitability of a patient to initially receive treatment with a TLR inhibitor, to predict the suitability of a patient to continue to receive treatment with a TLR inhibitor and to predict the likelihood of clinical benefits when a patient is treated with a TLR inhibitor.

Moreover, the present inventors found that the administration of a TLR inhibitor increased potency of glucocorticosteroids even in the context of IFN-α pre-treatment.

Thus, provided herein are methods that are based upon determining IFN-I activity for predicting therapeutic effectiveness of a TLR inhibitor in a patient, for predicting the suitability of an individual with a disease to initiate treatment with a TLR inhibitor, for predicting the suitability of a patient to continue treatment with a TLR inhibitor and/or for treating patients with a TLR inhibitor. Also, provided herein are methods that are based upon determining IFN-I activity for predicting therapeutic effectiveness of a TLR inhibitor and a corticosteroid in a patient, for predicting the suitability of an individual with a disease to initiate treatment with a TLR inhibitor and a corticosteroid, for predicting the suitability of a patient to continue treatment with a TLR inhibitor and a corticosteroid and/or for treating patients with a TLR inhibitor in combination with a corticosteroid.

Provided herein is a method for predicting therapeutic effectiveness of a TLR inhibitor in an individual with a disease comprising determining the IFN-I activity in a sample from the individual, wherein the IFN-I activity in the sample indicates the therapeutic effectiveness of the TLR inhibitor. In some embodiments, a TLR inhibitor is predicted to be therapeutically effective if the individual has high IFN-I activity. In some embodiments, the IFN-I activity is compared to a reference IFN-I activity and the TLR inhibitor is predicted to be therapeutically effective in the individual if the IFN-I activity of the individual is higher than the reference IFN-I activity and predicted not to be therapeutically effective if the IFN-I activity of the individual is lower than the reference IFN-I activity. In some embodiments, the reference IFN-I activity is chosen to divide those individuals in which therapeutic effectiveness is more likely from those individuals in which therapeutic effectiveness is less likely and, thus, therapeutic effectiveness is predicted for an individual if the IFN-I activity of the individual is higher than the reference IFN-I activity and predicted not to be therapeutically effective if the IFN-I activity of the individual is lower than the reference IFN-I activity. In some embodiments, the reference IFN-I activity is indicative for therapeutic effectiveness and, thus, therapeutic effectiveness is predicted for an individual if the IFN-I activity of the individual is around or above the reference IFN-I activity. In some embodiments, the reference IFN-I activity is indicative for therapeutic ineffectiveness and, thus, therapeutic ineffectiveness is predicted for an individual if the IFN-I activity of the individual is around or below the reference IFN-I activity.

Provided herein is a method for predicting therapeutic effectiveness of a TLR inhibitor in an individual with a disease comprising determining an IFN-I signature expression pattern in a sample from the individual, wherein the IFN-I signature expression pattern in the sample indicates the therapeutic effectiveness of the TLR inhibitor. In some embodiments, the IFN-I signature expression pattern is compared to one or more reference IFN-I signature expression patterns. In some embodiments, the reference IFN-I signature expression pattern is indicative for therapeutic effectiveness and, thus, therapeutic effectiveness is predicted by a correlation between the determined IFN-I signature expression pattern and a reference IFN-I signature expression pattern and a lack of therapeutic effectiveness is predicted by the absence of a correlation between the determined IFN-I signature expression pattern and the reference IFN-I signature expression pattern. In some embodiments, the reference IFN-I signature expression pattern is indicative for the lack of therapeutic effectiveness and, thus, therapeutic effectiveness is predicted by a lack of correlation between the determined IFN-I signature expression pattern and a reference IFN-I signature expression pattern and a lack of therapeutic effectiveness is predicted by a correlation between the determined IFN-I signature expression pattern and the reference IFN-I signature expression pattern. In some embodiments, there is a first reference IFN-I signature expression pattern that is indicative for the therapeutic effectiveness of the TLR inhibitor and a second reference IFN-I signature expression pattern that is indicative for the lack of therapeutic effectiveness of the TLR inhibitor and the TLR inhibitor is predicted to be therapeutically effective in the individual if there is a correlation between the determined IFN-I signature expression pattern and the first reference IFN-I signature expression pattern and the TLR inhibitor is predicted not to be therapeutically effective in the individual if there is a correlation between the determined IFN-I signature expression pattern and the second reference IFN-I signature expression pattern.

Also provided herein is a method for predicting therapeutic effectiveness of a TLR inhibitor in an individual with a disease comprising determining an IFN-I signature score in a sample from the individual, wherein the IFN-I signature score in the sample indicates therapeutic effectiveness. In some embodiments, the TLR inhibitor is predicted to be therapeutically effective in the individual if the determined IFN-I signature score is higher than a reference IFN-I signature score and predicted not to be therapeutically effective in the individual if the determined IFN-I signature score is lower than the reference IFN-I signature score. In some embodiments, the reference IFN-I signature score is chosen to divide those individuals in which therapeutic effectiveness is more likely from those individuals in which therapeutic effectiveness is less likely and, thus, therapeutic effectiveness is predicted for an individual if the IFN-I signature score of the individual is higher than the reference IFN-I signature score and predicted not to be therapeutically effective if the IFN-I signature score of the individual is lower than the reference IFN-I signature score. In some embodiments, the reference IFN-I signature score is indicative for therapeutic effectiveness and, thus, therapeutic effectiveness is predicted for an individual if the IFN-I signature score of the individual is around or above the reference IFN-I signature score. In some embodiments, the reference IFN-I signature score is indicative for therapeutic ineffectiveness and, thus, therapeutic ineffectiveness is predicted for an individual if the IFN-I signature score of the individual is around or below the reference IFN-I signature score.

In some embodiments of any method for predicting therapeutic effectiveness of a TLR inhibitor in an individual with a disease disclosed herein, if therapeutic effectiveness were predicted in such individual, the TLR inhibitor is administered to the individual.

Provided herein is a method for predicting the suitability of an individual with a disease to initiate treatment with a TLR inhibitor comprising determining the IFN-I activity in a sample from the individual, wherein the IFN-I activity in the sample indicates the suitability of the individual to initiate the treatment. In some embodiments, an individual having high IFN-I activity is assessed to be suitable for initiating the treatment. In some embodiments, the IFN-I activity is compared to a reference IFN-I activity and the individual is assessed to be suitable to initiate the treatment if the IFN-I activity of the individual is higher than the reference IFN-I activity and assessed to be unsuitable to initiate the treatment if the IFN-I activity of the individual is lower than the reference IFN-I activity. In some embodiments, the reference IFN-I activity is chosen to divide those individuals in which therapeutic effectiveness is more likely from those individuals in which therapeutic effectiveness is less likely and, thus, an individual is predicted to be suitable to initiate treatment with a TLR inhibitor if the IFN-I activity of the individual is higher than the reference IFN-I activity and predicted not to be suitable if the IFN-I activity of the individual is lower than the reference IFN-I activity. In some embodiments, the reference IFN-I activity is indicative for therapeutic effectiveness and, thus, an individual is predicted to be suitable to initiate treatment with a TLR inhibitor if the IFN-I activity of the individual is around or above the reference IFN-I activity. In some embodiments, the reference IFN-I activity is indicative for therapeutic ineffectiveness and, thus, an individual is predicted not to be suitable to initiate treatment with a TLR inhibitor if the IFN-I activity of the individual is around or below the reference IFN-I activity.

Provided herein is a method for predicting the suitability of an individual with a disease to initiate treatment with a TLR inhibitor comprising determining an IFN-I signature expression pattern in a sample from the individual, wherein the IFN-I signature expression pattern in the sample indicates the suitability of the individual to initiate the treatment. In some embodiments, the IFN-I signature expression pattern is compared to one or more reference IFN-I signature expression patterns. In some embodiments, the reference IFN-I signature expression pattern is indicative for the suitability to initiate treatment with a TLR inhibitor and, thus, the suitability to initiate treatment is indicated by a correlation between the determined IFN-I signature expression pattern and a reference IFN-I signature expression pattern and a lack of suitability to initiate treatment is indicated by the absence of a correlation between the determined IFN-I signature expression pattern and the reference IFN-I signature expression pattern. In some embodiments, the reference IFN-I signature expression pattern is indicative for the lack of suitability to initiate treatment with a TLR inhibitor and, thus, suitability to initiate treatment is indicated by a lack of correlation between the determined IFN-I signature expression pattern and a reference IFN-I signature expression pattern and a lack of suitability to initiate treatment is indicated by a correlation between the determined IFN-I signature expression pattern and the reference IFN-I signature expression pattern. In some embodiments, there is a first reference IFN-I signature expression pattern that is indicative for the suitability of the individual to initiate the treatment with the TLR inhibitor and a second reference IFN-I signature expression pattern that is indicative for the unsuitability of the individual to initiate the treatment with the TLR inhibitor and the individual is assessed to be suitable to initiate the treatment if there is a correlation between the determined IFN-I signature expression pattern and the first reference IFN-I signature expression pattern and the individual is assessed to be unsuitable to initiate the treatment if there is a correlation between the determined IFN-I signature expression pattern and the second reference IFN-I signature expression pattern.

Also provided herein is a method for predicting the suitability of an individual with a disease to initiate treatment with a TLR inhibitor comprising determining an IFN-I signature score in a sample from the individual, wherein the IFN-I signature score in the sample indicates the suitability of the individual to initiate the treatment. In some embodiments, the individual is assessed to be suitable to initiate the treatment if the determined IFN-I signature score is higher than a reference IFN-I signature score and unsuitable to initiate the treatment if the determined IFN-I signature score is lower than the reference IFN-I signature score. In some embodiments of any method for predicting the suitability of an individual with a disease to initiate treatment with a TLR inhibitor disclosed herein, if such individual is assessed to be suitable for initiating the treatment, the TLR inhibitor is administered to the individual. In some embodiments, the reference IFN-I signature score is chosen to divide those individuals in which therapeutic effectiveness is more likely from those individuals in which therapeutic effectiveness is less likely and, thus, an individual is predicted to be suitable to initiate treatment with a TLR inhibitor if the IFN-I signature score of the individual is higher than the reference IFN-I signature score and predicted not to be suitable if the IFN-I signature score of the individual is lower than the reference IFN-I signature score. In some embodiments, the reference IFN-I signature score is indicative for therapeutic effectiveness and, thus, an individual is predicted to be suitable to initiate treatment with a TLR inhibitor if the IFN-I signature score of the individual is around or above the reference IFN-I signature score. In some embodiments, the reference IFN-I signature score is indicative for therapeutic ineffectiveness and, thus, an individual is predicted not to be suitable to initiate treatment with a TLR inhibitor if the IFN-I signature score of the individual is around or below the reference IFN-I signature score.

Provided herein is a method for predicting the suitability of an individual with a disease who is receiving treatment with a TLR inhibitor to continue the treatment comprising determining the IFN-I activity in a sample from the individual, wherein the IFN-I activity in the sample indicates the suitability of the individual to continue the treatment. In some embodiments, an individual having high IFN-I activity is assessed to be suitable to continue the treatment. In some embodiments, the IFN-I activity is compared to a reference IFN-I activity and the individual is assessed to be suitable to continue the treatment if the IFN-I activity of the individual is higher than the reference IFN-I activity and unsuitable to continue the treatment if the IFN-I activity of the individual is lower than the reference IFN-I activity. In some embodiments, the reference IFN-I activity is chosen to divide those individuals in which therapeutic effectiveness is more likely from those individuals in which therapeutic effectiveness is less likely and, thus, an individual is predicted to be suitable to continue treatment with a TLR inhibitor if the IFN-I activity of the individual is higher than the reference IFN-I activity and predicted not to be suitable if the IFN-I activity of the individual is lower than the reference IFN-I activity. In some embodiments, the reference IFN-I activity is indicative for therapeutic effectiveness and, thus, an individual is predicted to be suitable to continue treatment with a TLR inhibitor if the IFN-I activity of the individual is around or above the reference IFN-I activity. In some embodiments, the reference IFN-I activity is indicative for therapeutic ineffectiveness and, thus, an individual is predicted not to be suitable to continue treatment with a TLR inhibitor if the IFN-I activity of the individual is around or below the reference IFN-I activity.

Provided herein is a method for predicting the suitability of an individual with a disease who is receiving treatment with a TLR inhibitor to continue the treatment comprising determining an IFN-I signature expression pattern in a sample from the individual, wherein the IFN-I signature expression pattern in the sample indicates the suitability of the individual to continue the treatment. In some embodiments, the IFN-I signature expression pattern is compared to one or more reference IFN-I signature expression patterns. In some embodiments, the reference IFN-I signature expression pattern is indicative for the suitability to continue treatment with a TLR inhibitor and, thus, suitability to continue the treatment is indicated by a correlation between the determined IFN-I signature expression pattern and a reference IFN-I signature expression pattern and a lack of suitability to continue the treatment is indicated by the absence of a correlation between the determined IFN-I signature expression pattern and the reference IFN-I signature expression pattern. In some embodiments, the reference IFN-I signature expression pattern is indicative for the lack of suitability to continue treatment with a TLR inhibitor and, thus, suitability to continue the treatment is indicated by a lack of correlation between the determined IFN-I signature expression pattern and a reference IFN-I signature expression pattern and a lack of suitability to continue the treatment is indicated by a correlation between the determined IFN-I signature expression pattern and the reference IFN-I signature expression pattern. In some embodiments, there is a first reference IFN-I signature expression pattern that is indicative for the suitability of the individual to continue the treatment with the TLR inhibitor and a second reference IFN-I signature expression pattern that is indicative for the unsuitability of the individual to continue the treatment with the TLR inhibitor and the individual is assessed to be suitable to continue the treatment if there is a correlation between the determined IFN-I signature expression pattern and the first reference IFN-I signature expression pattern and the individual is assessed to be unsuitable to continue the treatment if there is a correlation between the determined IFN-I signature expression pattern and the second reference IFN-I signature expression pattern.

Also provided herein is a method for predicting the suitability of an individual with a disease who is receiving treatment with a TLR inhibitor to continue said treatment comprising determining an IFN-I signature score in a sample from the individual, wherein the IFN-I signature score in the sample indicates the suitability of the individual to continue the treatment. In some embodiments, the individual is assessed to be suitable to continue the treatment if the determined IFN-I signature score is higher than a reference IFN-I signature score and unsuitable to continue the treatment if the determined IFN-I signature score is lower than the reference IFN-I signature score. In some embodiments of any method for predicting the suitability of an individual with a disease who is receiving treatment with a TLR inhibitor to continue the treatment disclosed herein, if such individual is assessed to be suitable for continuing the treatment, the TLR inhibitor is administered to the individual. In some embodiments, the reference IFN-I signature score is chosen to divide those individuals in which therapeutic effectiveness is more likely from those individuals in which therapeutic effectiveness is less likely and, thus, an individual is predicted to be suitable to continue treatment with a TLR inhibitor if the IFN-I signature score of the individual is higher than the reference IFN-I signature score and predicted not to be suitable if the IFN-I signature score of the individual is lower than the reference IFN-I signature score. In some embodiments, the reference IFN-I signature score is indicative for therapeutic effectiveness and, thus, an individual is predicted to be suitable to continue treatment with a TLR inhibitor if the IFN-I signature score of the individual is around or above the reference IFN-I signature score. In some embodiments, the reference IFN-I signature score is indicative for therapeutic ineffectiveness and, thus, an individual is predicted not to be suitable to continue treatment with a TLR inhibitor if the IFN-I signature score of the individual is around or below the reference IFN-I signature score.

Provided herein is a TLR inhibitor for use in a method of treating a disease in an individual comprising administering the TLR inhibitor to the individual, wherein treatment is based upon IFN-I acitivity in a sample from the individual. In some embodiments, an individual having high IFN-I activity is administered the TLR inhibitor. In some embodiments, the IFN-I activity is compared to a reference IFN-I activity and the individual is administered the TLR inhibitor if the IFN-I activity of the individual is higher than the reference IFN-I activity. In some embodiments, the reference IFN-I activity is chosen to divide those individuals in which therapeutic effectiveness is more likely from those individuals in which therapeutic effectiveness is less likely and, thus, an individual is administered the TLR inhibitor if the IFN-I activity of the individual is higher than the reference IFN-I activity and not administered the TLR inhibitor if the IFN-I activity of the individual is lower than the reference IFN-I activity. In some embodiments, the reference IFN-I activity is indicative for therapeutic effectiveness and, thus, an individual is administered the TLR inhibitor if the IFN-I activity of the individual is around or above the reference IFN-I activity. In some embodiments, the reference IFN-I activity is indicative for therapeutic ineffectiveness and, thus, an individual is not administered the TLR inhibitor if the IFN-I activity of the individual is around or below the reference IFN-I activity.

Provided herein is a TLR inhibitor for use in a method of treating a disease in an individual having high IFN-I activity comprising administering the TLR inhibitor to the individual. Provided herein is a TLR inhibitor for use in a method of treating a disease in an individual comprising administering the TLR inhibitor to the individual, wherein treatment is based upon an IFN-I signature expression pattern in a sample from the individual. In some embodiments, the IFN-I signature expression pattern is compared to one or more reference IFN-I signature expression patterns. In some embodiments, the reference IFN-I signature expression pattern is indicative for the administration of the TLR inhibitor and, thus, the TLR inhibitor is administered if there is a correlation between the determined IFN-I signature expression pattern and a reference IFN-I signature expression pattern and the TLR inhibitor is not administered if there is no correlation between the determined IFN-I signature expression pattern and the reference IFN-I signature expression pattern. In some embodiments, the reference IFN-I signature expression pattern is indicative for not administering the TLR inhibitor and, thus, the TLR inhibitor is administered if there is a lack of correlation between the determined IFN-I signature expression pattern and the TLR inhibitor is not administered if there is a correlation between the determined IFN-I signature expression pattern and the reference IFN-I signature expression pattern. In some embodiments, there is a first reference IFN-I signature expression pattern that is indicative for the administration of the TLR inhibitor and a second reference IFN-I signature expression pattern that is indicative for not administering the TLR inhibitor and the TLR inhibitor is administered if there is a correlation between the determined IFN-I signature expression pattern and the first reference IFN-I signature expression pattern and the TLR inhibitor is not administered if there is a correlation between the determined IFN-I signature expression pattern and the second reference IFN-I signature expression pattern.

Also provided herein is a TLR inhibitor for use in a method of treating a disease in an individual comprising administering the TLR inhibitor to the individual, wherein treatment is based upon an IFN-I signature score in a sample from the individual. In some embodiments, the TLR inhibitor is administered to the individual if the IFN-I signature score in the sample of the individual is higher than a reference IFN-I signature score. Also provided herein is a TLR inhibitor for use in a method of treating a disease in an individual comprising determining an IFN-I signature score in a sample from the individual and administering a TLR inhibitor to the individual if the IFN-I signature score is higher than a reference IFN-I signature score. In some embodiments, the reference IFN-I signature score is chosen to divide those individuals in which therapeutic effectiveness is more likely from those individuals in which therapeutic effectiveness is less likely and, thus, an individual is administered the TLR inhibitor if the IFN-I signature score of the individual is higher than the reference IFN-I signature score and not administered the TLR inhibitor if the IFN-I signature score of the individual is lower than the reference IFN-I signature score. In some embodiments, the reference IFN-I signature score is indicative for therapeutic effectiveness and, thus, an individual is administered the TLR inhibitor if the IFN-I signature score of the individual is around or above the reference IFN-I signature score. In some embodiments, the reference IFN-I signature score is indicative for therapeutic ineffectiveness and, thus, an individual is administered the TLR inhibitor if the IFN-I signature score of the individual is around or below the reference IFN-I signature score.

Also provided herein is a TLR inhibitor for use in a method of treating a disease in an individual comprising selecting a patient having an IFN-I signature score above a reference IFN-I signature score and administering the TLR inhibitor to the individual. Also provided herein is a TLR inhibitor for use in a method of treating a disease in an individual comprising selecting a patient having an IFN-I signature score above a reference IFN-I signature score, which is chosen to divide those individuals in which therapeutic effectiveness is more likely from those individuals in which therapeutic effectiveness is less likely, and administering the TLR inhibitor to the individual. Also provided herein is a TLR inhibitor for use in a method of treating a disease in an individual comprising administering the TLR inhibitor to the individual, wherein the individual has a higher IFN-I signature score in a sample from the individual as compared to a reference IFN-I signature score. Also provided herein is a TLR inhibitor for use in a method of treating a disease in an individual comprising administering the TLR inhibitor to the individual, wherein the individual has a higher IFN-I signature score in a sample from the individual as compared to a reference IFN-I signature score, which is chosen to divide those individuals in which therapeutic effectiveness is more likely from those individuals in which therapeutic effectiveness is less likely.

Provided herein is a method of treating a disease in an individual comprising administering a TLR inhibitor to the individual, wherein treatment is based upon IFN-I acitivity in a sample from the individual. In some embodiments, an individual having high IFN-I activity is administered the TLR inhibitor. In some embodiments, the IFN-I activity is compared to a reference IFN-I activity and the individual is administered the TLR inhibitor if the IFN-I activity of the individual is higher than the reference IFN-I activity. Provided herein is a method of treating a disease in an individual having high IFN-I activity comprising administering a TLR inhibitor to the individual. Provided herein is a method of treating a disease in an individual comprising administering a TLR inhibitor to the individual, wherein treatment is based upon an IFN-I signature expression pattern in a sample from the individual. In some embodiments, the IFN-I signature expression pattern is compared to one or more reference IFN-I signature expression patterns. In some embodiments, the reference IFN-I signature expression pattern is indicative for the administration of the TLR inhibitor and, thus, the TLR inhibitor is administered if there is a correlation between the determined IFN-I signature expression pattern and a reference IFN-I signature expression pattern and the TLR inhibitor is not administered if there is no correlation between the determined IFN-I signature expression pattern and the reference IFN-I signature expression pattern. In some embodiments, the reference IFN-I signature expression pattern is indicative for not administering the TLR inhibitor and, thus, the TLR inhibitor is administered if there is a lack of correlation between the determined IFN-I signature expression pattern and the TLR inhibitor is not administered if there is a correlation between the determined IFN-I signature expression pattern and the reference IFN-I signature expression pattern. In some embodiments, there is a first reference IFN-I signature expression pattern that is indicative for the administration of the TLR inhibitor and a second reference IFN-I signature expression pattern that is indicative for not administering the TLR inhibitor and the TLR inhibitor is administered if there is a correlation between the determined IFN-I signature expression pattern and the first reference IFN-I signature expression pattern and the TLR inhibitor is not administered if there is a correlation between the determined IFN-I signature expression pattern and the second reference IFN-I signature expression pattern. Also provided herein is a method of treating a disease in an individual comprising administering a TLR inhibitor to the individual, wherein treatment is based upon an IFN-I signature score in a sample from the individual. In some embodiments, the TLR inhibitor is administered to the individual if the IFN-I signature score in the sample of the individual is higher than a reference IFN-I signature score. Also provided herein is a method of treating a disease in an individual comprising determining an IFN-I signature score in a sample from the individual and administering a TLR inhibitor to the individual if the IFN-I signature score is higher than a reference IFN-I signature score. Also provided herein is a method of treating a disease in an individual comprising selecting a patient having an IFN-I signature score above a reference IFN-I signature score and administering a TLR inhibitor to the individual. Also provided herein is a method of treating a disease in an individual comprising administering a TLR inhibitor to the individual, wherein the individual has a higher IFN-I signature score in a sample from the individual as compared to a reference IFN-I signature score.

Provided herein is the use of a TLR inhibitor to treat a disease in an individual comprising administering the TLR inhibitor to the individual, wherein treatment is based upon IFN-I acitivity in a sample from the individual. In some embodiments, an individual having high IFN-I activity is administered the TLR inhibitor. In some embodiments, the IFN-I activity is compared to a reference IFN-I activity and the individual is administered the TLR inhibitor if the IFN-I activity of the individual is higher than the reference IFN-I activity. Provided herein is the use of a TLR inhibitor to treat a disease in an individual having high IFN-I activity comprising administering the TLR inhibitor to the individual. Provided herein is the use of a TLR inhibitor to treat a disease in an individual comprising administering the TLR inhibitor to the individual, wherein treatment is based upon an IFN-I signature expression pattern in a sample from the individual. In some embodiments, the IFN-I signature expression pattern is compared to one or more reference IFN-I signature expression patterns. In some embodiments, the reference IFN-I signature expression pattern is indicative for the administration of the TLR inhibitor and, thus, the TLR inhibitor is administered if there is a correlation between the determined IFN-I signature expression pattern and a reference IFN-I signature expression pattern and the TLR inhibitor is not administered if there is no correlation between the determined IFN-I signature expression pattern and the reference IFN-I signature expression pattern. In some embodiments, the reference IFN-I signature expression pattern is indicative for not administering the TLR inhibitor and, thus, the TLR inhibitor is administered if there is a lack of correlation between the determined IFN-I signature expression pattern and the TLR inhibitor is not administered if there is a correlation between the determined IFN-I signature expression pattern and the reference IFN-I signature expression pattern. In some embodiments, there is a first reference IFN-I signature expression pattern that is indicative for the administration of the TLR inhibitor and a second reference IFN-I signature expression pattern that is indicative for not administering the TLR inhibitor and the TLR inhibitor is administered if there is a correlation between the determined IFN-I signature expression pattern and the first reference IFN-I signature expression pattern and the TLR inhibitor is not administered if there is a correlation between the determined IFN-I signature expression pattern and the second reference IFN-I signature expression pattern. Also provided herein is the use of a TLR inhibitor to treat a disease in an individual comprising administering a TLR inhibitor to the individual, wherein treatment is based upon an IFN-I signature score in a sample from the individual. In some embodiments, the TLR inhibitor is administered to the individual if the IFN-I signature score in the sample of the individual is higher than a reference IFN-I signature score. Also provided herein is the use of a TLR inhibitor to treat a disease in an individual comprising determining an IFN-I signature score in a sample from the individual and administering the TLR inhibitor to the individual if the IFN-I signature score is higher than a reference IFN-I signature score. Also provided herein is the use of a TLR inhibitor to treat a disease in an individual comprising selecting a patient having an IFN-I signature score above a reference IFN-I signature score and administering the TLR inhibitor to the individual. Also provided herein is the use of a TLR inhibitor to treat a disease in an individual comprising administering the TLR inhibitor to the individual, wherein the individual has a higher IFN-I signature score in a sample from the individual as compared to a reference IFN-I signature score.

Provided herein is the use of a TLR inhibitor for the manufacture of a medicament to treat a disease in an individual comprising administering the TLR inhibitor to the individual, wherein treatment is based upon IFN-I acitivity in a sample from the individual. In some embodiments, an individual having high IFN-I activity is administered the TLR inhibitor. In some embodiments, the IFN-I activity is compared to a reference IFN-I activity and the individual is administered the TLR inhibitor if the IFN-I activity of the individual is higher than the reference IFN-I activity. Provided herein is the use of a TLR inhibitor for the manufacture of a medicament to treat a disease in an individual having high IFN-I activity comprising administering the TLR inhibitor to the individual. Provided herein is the use of a TLR inhibitor for the manufacture of a medicament to treat a disease in an individual comprising administering the TLR inhibitor to the individual, wherein treatment is based upon an IFN-I signature expression pattern in a sample from the individual. In some embodiments, the IFN-I signature expression pattern is compared to one or more reference IFN-I signature expression patterns. In some embodiments, the reference IFN-I signature expression pattern is indicative for the administration of the TLR inhibitor and, thus, the TLR inhibitor is administered if there is a correlation between the determined IFN-I signature expression pattern and a reference IFN-I signature expression pattern and the TLR inhibitor is not administered if there is no correlation between the determined IFN-I signature expression pattern and the reference IFN-I signature expression pattern. In some embodiments, the reference IFN-I signature expression pattern is indicative for not administering the TLR inhibitor and, thus, the TLR inhibitor is administered if there is a lack of correlation between the determined IFN-I signature expression pattern and the TLR inhibitor is not administered if there is a correlation between the determined IFN-I signature expression pattern and the reference IFN-I signature expression pattern. In some embodiments, there is a first reference IFN-I signature expression pattern that is indicative for the administration of the TLR inhibitor and a second reference IFN-I signature expression pattern that is indicative for not administering the TLR inhibitor and the TLR inhibitor is administered if there is a correlation between the determined IFN-I signature expression pattern and the first reference IFN-I signature expression pattern and the TLR inhibitor is not administered if there is a correlation between the determined IFN-I signature expression pattern and the second reference IFN-I signature expression pattern. Also provided herein is the use of a TLR inhibitor for the manufacture of a medicament to treat a disease in an individual comprising administering the TLR inhibitor to the individual, wherein treatment is based upon an IFN-I signature score in a sample from the individual. Also provided herein is the use of a TLR inhibitor for the manufacture of a medicament to treat a disease in an individual comprising determining an IFN-I signature score in a sample from the individual and administering the TLR inhibitor to the individual if the IFN-I signature score is higher than a reference IFN-I signature score. Also provided herein is the use of a TLR inhibitor for the manufacture of a medicament to treat a disease in an individual comprising selecting a patient having an IFN-I signature score above a reference IFN-I signature score and administering the TLR inhibitor to the individual. Also provided herein is the use of a TLR inhibitor for the manufacture of a medicament to treat a disease in an individual comprising administering the TLR inhibitor to the individual, wherein the individual has a higher IFN-I signature score in a sample from the individual as compared to a reference IFN-I signature score.

The present disclosure also provides the following uses of a TLR inhibitor in combination with a corticosteroid.

Provided herein are a TLR inhibitor and a corticosteroid, such as a TLR7 and/or TLR8 inhibitor and a glucocorticosteroid, for use in a method of treating a disease in an individual having high IFN-I activity, wherein the method comprises administering the TLR inhibitor and the corticosteroid to the individual. Provided herein are a TLR inhibitor and a corticosteroid, such as a TLR7 and/or TLR8 inhibitor and a glucocorticosteroid, for use in a method of treating a disease in an individual having an IFN-I activity that is higher than a reference IFN-I activity, wherein the method comprises administering the TLR inhibitor and the corticosteroid to the individual. Provided herein is a TLR inhibitor and a corticosteroid, such as a TLR7 and/or TLR8 inhibitor and a glucocorticosteroid, for use in a method of treating a disease in an individual comprising (i) determining the IFN-I activity of the individual, (ii) comparing the determined IFN-I activity to a reference IFN-I activity and (iii), depending on the result of the comparison, administering the TLR inhibitor and the corticosteroid to the individual. In some embodiments, the reference IFN-I activity is chosen to divide those individuals in which therapeutic effectiveness of the TLR inhibitor is more likely from those individuals in which therapeutic effectiveness of the TLR inhibitor is less likely and, thus, an individual is administered the TLR inhibitor and the corticosteroid if the IFN-I activity of the individual is higher than the reference IFN-I activity and not administered the TLR inhibitor and the corticosteroid if the IFN-I activity of the individual is lower than the reference IFN-I activity. In some embodiments, the reference IFN-I activity is indicative for therapeutic effectiveness of the TLR inhibitor and, thus, an individual is administered the TLR inhibitor and the corticosteroid if the IFN-I activity of the individual is around or above the reference IFN-I activity. In some embodiments, the reference IFN-I activity is indicative for therapeutic ineffectiveness of the TLR inhibitor and, thus, an individual is not administered the TLR inhibitor and the corticosteroid if the IFN-I activity of the individual is around or below the reference IFN-I activity. Provided herein is a TLR inhibitor and a corticosteroid, such as a TLR7 and/or TLR8 inhibitor and a glucocorticosteroid, for use in a method of treating a disease in an individual comprising administering the TLR inhibitor and the corticosteroid to the individual, wherein the individual has a higher IFN-I signature score than a reference IFN-I signature score. Provided herein is a TLR inhibitor and a corticosteroid, such as a TLR7 and/or TLR8 inhibitor and a glucocorticosteroid, for use in a method of treating a disease in an individual comprising (i) determining the IFN-I signature score of the individual, (ii) comparing the determined IFN-I signature score to a reference IFN-I signature score and (iii), depending on the result of the comparison, administering the TLR inhibitor and the corticosteroid to the individual. In some embodiments, the reference IFN-I signature score is chosen to divide those individuals in which therapeutic effectiveness of the TLR inhibitor is more likely from those individuals in which therapeutic effectiveness of the TLR inhibitor is less likely and, thus, an individual is administered the TLR inhibitor and the corticosteroid if the IFN-I signature score of the individual is higher than the reference IFN-I signature score and not administered the TLR inhibitor and the corticosteroid if the IFN-I signature score of the individual is lower than the reference IFN-I signature score. In some embodiments, the reference IFN-I signature score is indicative for therapeutic effectiveness of the TLR inhibitor and, thus, an individual is administered the TLR inhibitor and the corticosteroid if the IFN-I signature score of the individual is around or above the reference IFN-I signature score. In some embodiments, the reference IFN-I signature score is indicative for therapeutic ineffectiveness of the TLR inhibitor and, thus, an individual is not administered the TLR inhibitor and the corticosteroid if the IFN-I signature score of the individual is around or below the reference IFN-I signature score. In some embodiments, the IFN-I activity or IFN-I signature score of the treated individual is determined in a sample from the individual. In some embodiments, a reduced effective amount (including, but not limited to, dosage volume, dosage concentration, and/or total drug dose administered) of the corticosteroid is administered when administered together with the TLR inhibitor.

Provided herein is a method of treating a disease in an individual having high IFN-I activity comprising administering a TLR inhibitor and a corticosteroid, such as a TLR7 and/or

TLR8 inhibitor and a glucocorticosteroid, to the individual. Provided herein is a method of treating a disease in an individual having an IFN-I activity that is higher than a reference IFN-I activity, wherein the method comprises administering a TLR inhibitor and a corticosteroid, such as a TLR7 and/or TLR8 inhibitor and a glucocorticosteroid, to the individual. Provided herein is a method of treating a disease in an individual comprising administering a TLR inhibitor and a corticosteroid, such as a TLR7 and/or TLR8 inhibitor and a glucocorticosteroid, to the individual, wherein the individual has a higher IFN-I signature score than a reference IFN-I signature score. In some embodiments, the IFN-I activity or IFN-I signature score of the treated individual is determined in a sample from the individual. In some embodiments, a reduced effective amount (including, but not limited to, dosage volume, dosage concentration, and/or total drug dose administered) of the corticosteroid is administered when administered together with the TLR inhibitor.

Provided herein is the use of a TLR inhibitor and a corticosteroid, such as a TLR7 and/or TLR8 inhibitor and a glucocorticosteroid, to treat a disease in an individual having high IFN-I activity comprising administering the TLR inhibitor and the corticosteroid to the individual. Provided herein is the use of a TLR inhibitor and a corticosteroid, such as a TLR7 and/or TLR8 inhibitor and a glucocorticosteroid, to treat a disease in an individual having an IFN-I activity that is higher than a reference IFN-I activity, wherein the method comprises administering the TLR inhibitor and the corticosteroid to the individual. Provided herein is the use of a TLR inhibitor and a corticosteroid, such as a TLR7 and/or TLR8 inhibitor and a glucocorticosteroid, to treat a disease in an individual comprising administering the TLR inhibitor and the corticosteroid to the individual, wherein the individual has a higher IFN-I signature score than a reference IFN-I signature score. In some embodiments, the IFN-I activity or IFN-I signature score of the treated individual is determined in a sample from the individual. In some embodiments, a reduced effective amount (including, but not limited to, dosage volume, dosage concentration, and/or total drug dose administered) of the corticosteroid is administered when administered together with the TLR inhibitor.

Provided herein is the use of a TLR inhibitor and a corticosteroid, such as a TLR7 and/or TLR8 inhibitor and a glucocorticosteroid, for the manufacture of a medicament to treat a disease in an individual having high IFN-I activity comprising administering the TLR inhibitor and the corticosteroid to the individual. Provided herein is the use of a TLR inhibitor and a corticosteroid, such as a TLR7 and/or TLR8 inhibitor and a glucocorticosteroid, for the manufacture of a medicament to treat a disease in an individual having an IFN-I activity that is higher than a reference IFN-I activity, wherein the method comprises administering the TLR inhibitor and the corticosteroid to the individual. Provided herein is the use of a TLR inhibitor and a corticosteroid, such as a TLR7 and/or TLR8 inhibitor and a glucocorticosteroid, for the manufacture of a medicament to treat a disease in an individual comprising administering the TLR inhibitor and the corticosteroid to the individual, wherein the individual has a higher IFN-I signature score than a reference IFN-I signature score. In some embodiments, the IFN-I activity or IFN-I signature score of the treated individual is determined in a sample from the individual. In some embodiments, a reduced effective amount (including, but not limited to, dosage volume, dosage concentration, and/or total drug dose administered) of the corticosteroid is administered when administered together with the TLR inhibitor.

In some embodiments of any of the methods or uses herein that relate to the IFN-I activity in an individual, determining the IFN-I activity comprises (i) obtaining a sample from the individual; (ii) measuring the IFN-I activity in the sample; and (iii) normalizing the IFN-I activity.

In some embodiments of any of the methods or uses herein that relate to the IFN-I signature expression pattern in an individual, determining the IFN-I signature expression pattern comprises (i) obtaining a sample from the individual; (ii) measuring the expression level for each gene in the IFN-I signature in the sample; and (iii) normalizing each of the gene expression levels to obtain the IFN-I signature expression pattern.

In some embodiments of any of the methods or uses herein that relate to the IFN-I signature score in an individual, determining the IFN-I signature score comprises (i) obtaining a sample from the individual; (ii) measuring the expression level for each gene in the IFN-I signature in the sample; (iii) normalizing each of the gene expression levels; and (iii) calculating the arithmetic mean of the normalized gene expression levels to obtain the IFN-I signature score.

In certain embodiments of any of the methods or uses disclosed herein, the sample is a blood sample or a tissue sample. In certain embodiments of any of the methods or uses disclosed herein, the sample comprises peripheral blood mononuclear cells (PBMC) and/or skin tissue. In some embodiments, the sample is extracted RNA.

The expression levels of the genes in the IFN-I signature may be measured at the mRNA or the protein level. In certain embodiments of any of the methods or uses disclosed herein, the gene expression level of the IFN-I signature is measured by measuring the mRNA levels of the genes of the IFN-I signature. In certain embodiments of any of the methods or uses disclosed herein, the gene expression level of the IFN-I signature is measured by measuring the protein levels of the genes of the IFN-I signature.

Methods for measuring mRNA and protein levels are well known in the art. For instance, mRNA expression levels may be determined using Northern blotting, quantitative polymerase chain reaction (qPCR) or microarray and protein expression levels may be determined using ELISA, Western blotting and mass spectrometry.

In some embodiments, the measured expression level is normalized. For instance, the expression level may be normalized against the expression level of a gene which is known not to change significantly in expression among different samples. Genes that are commonly used for normalization include housekeeping genes, such as GAPDH, ACTB, TFRC, UBC and SDHA. In some embodiments, the genes ACTB, GAPDH and TFRC are used for normalization.

Certain embodiments disclosed herein refer to a reference IFN-I activity, reference IFN-I signature expression pattern or reference IFN-I signature score, which may be derived, for instance, from a second individual with or without the disease or from a population of individuals with or without the disease. In some embodiments, the reference IFN-I activity, reference IFN-I signature expression pattern or reference IFN-I signature score is determined retrospectively based on the IFN-I activity, IFN-I signature expression pattern or IFN-I signature score, respectively, in a patient population before treatment with the TLR inhibitor and the treatment outcome. The patient population may then be divided into two groups, one that showed a certain treatment outcome and one that did not. In some embodiments, the IFN-I activity or IFN-I signature score in between these two groups is defined as the reference IFN-I activity or reference IFN-I signature score, respectively. In other embodiments, the IFN-I activity or IFN-I signature score that is characteristic of one of the two groups is defined as the reference IFN-I activity or reference IFN-I signature score, respectively. In some embodiments, the IFN-I signature expression pattern that is characteristic for one or both groups is defined as the reference IFN-I signature expression pattern(s). Individuals having an IFN-I activity or IFN-I signature score that is comparable to one of the two groups (the group with or without the therapeutic effect) can then be defined as individuals where TLR inhibitor treatment is predicted to be therapeutically effective or as individuals where TLR inhibitor treatment is predicted to be therapeutically ineffective. In an alternative embodiment, the reference IFN-I activity, reference IFN-I signature expression pattern or reference IFN-I signature score is defined independently of a treatment outcome. For instance, in case of a patient population with a bimodal distribution of IFN-I activity or IFN-I signature scores, the reference IFN-I activity or reference IFN-I signature score may be defined as the IFN-I activity or IFN-I signature score that falls within the trough of the bimodal distribution, respectively, or it may be defined as the IFN-I activity or IFN-I signature score that is characteristic of one of the two groups, respectively. Moreover, a reference IFN-I activity or reference IFN-I signature score that is reflective of each of the two groups may be defined. Individuals having an IFN-I activity or IFN-I signature score that is comparable to one of the two groups can then be defined as having high IFN-I activity, where TLR inhibitor treatment is predicted to be therapeutically effective, or as having low IFN-I activity, where TLR inhibitor treatment is predicted to be therapeutically ineffective. Similarly, the IFN-I signature expression pattern that is characteristic for the group having lower IFN-I activity and/or having higher IFN-I activity in such population with a bimodal distribution may be defined as the reference IFN-I signature expression pattern(s). In one embodiment, in case of a population of healthy individuals, the reference IFN-I activity or reference IFN-I signature score may be defined as the IFN-I activity or IFN-I signature score around the upper limit of the IFN-I activity range or IFN-I signature score range for this population. In another embodiment, the IFN-I activity or IFN-I signature score of a healthy individual may be defined as the reference IFN-I activity and reference IFN-I signature score, respectively.

In some embodiments, the reference IFN-I activity is defined as follows:

(i) Defining a population of healthy individuals;

(ii) Determining the IFN-I activity in a sample from each individual in the population; and

(iii) Defining the IFN-I activity around the upper limit of the determined IFN-I activity range in the population as the reference IFN-I activity.

Such reference IFN-I activity may then be used to distinguish individuals in which therapeutic effectiveness is more likely (IFN-I activity above the reference IFN-I activity) from those individuals in which therapeutic effectiveness is less likely (IFN-I activity below the reference IFN-I activity).

In some embodiments, the reference IFN-I activity is defined as follows:
(i) Defining a population of patients having the same disease as the individual whose IFN-I activity is determined according to the methods or uses of the invention and who are treated with the same TLR inhibitor as said individual;
(ii) Determining the IFN-I activity in a sample from each patient in the patient population before treatment with the TLR inhibitor;
(iii) Determining therapeutic effectiveness for each patient in the patient population after treatment with the TLR inhibitor;
(iv) Dividing the patient population into a group that shows more therapeutic effectiveness and a group that shows less therapeutic effectiveness; and
(v) Defining the IFN-I activity that divides the two patient groups as the reference IFN-I activity.

Such reference IFN-I activity may then be used to distinguish individuals in which therapeutic effectiveness is more likely (IFN-I activity above the reference IFN-I activity) from those individuals in which therapeutic effectiveness is less likely (IFN-I activity below the reference IFN-I activity).

In some embodiments, the reference IFN-I activity is defined as follows:
(vi) Defining a population of patients having the same disease as the individual whose IFN-I activity is determined according to the methods or uses of the invention and who are treated with the same TLR inhibitor as said individual;
(vii) Determining the IFN-I activity in a sample from each patient in the patient population before treatment with the TLR inhibitor;
(viii) Determining therapeutic effectiveness for each patient in the patient population after treatment with the TLR inhibitor;
(ix) Dividing the patient population into a group that shows more therapeutic effectiveness and a group that shows less therapeutic effectiveness; and
(x) Defining the IFN-I activity that is characteristic for one or both of the two patient groups as the reference IFN-I activity.

Such reference IFN-I activity may then be used to identify those individuals in which therapeutic effectiveness is more likely (if the IFN-I activity characteristic for the patient group showing more therapeutic effectiveness was chosen as the reference IFN-I activity) and/or those individuals in which therapeutic effectiveness is less likely (if the IFN-I activity characteristic for the patient group showing less therapeutic effectiveness was chosen as the reference IFN-I activity).

In some embodiments, the reference IFN-I signature expression pattern is defined as follows:
(i) Defining a population of healthy individuals;
(ii) Determining the IFN-I signature expression pattern in a sample from each individual in the population; and
(iii) Defining a characteristic IFN-I signature expression pattern for the population as the reference IFN-I signature expression pattern.

Such reference IFN-I signature expression pattern derived from a healthy population may then be indicative of a lack of therapeutic effectiveness.

In some embodiments, the reference IFN-I signature expression pattern is defined as follows:
(i) Defining a population of patients having the same disease as the individual whose IFN-I signature expression pattern is determined according to the methods or uses of the invention and who are treated with the same TLR inhibitor as said individual;
(ii) Determining the IFN-I signature expression pattern in a sample from each patient in the patient population before treatment with the TLR inhibitor, wherein the IFN-I signature is the same IFN-I signature as the IFN-I signature of the individual whose IFN-I signature expression pattern is determined according to the methods or uses of the invention;
(iii) Determining therapeutic effectiveness for each patient in the patient population after treatment with the TLR inhibitor;
(iv) Dividing the patient population into a group that shows more therapeutic effectiveness and a group that shows less therapeutic effectiveness; and
(v) Defining a characteristic IFN-I signature expression pattern for one or both patient groups as the reference IFN-I signature expression pattern(s).

Such reference IFN-I signature expression pattern(s) derived from a patient population may then be indicative for the presence or lack of therapeutic effectiveness, respectively.

In some embodiments, the reference IFN-I signature score is defined as follows:

(iv) Defining a population of healthy individuals;

(v) Determining the IFN-I signature score in a sample from each individual in the population; and

(vi) Defining the IFN-I signature score around the upper limit of the determined IFN-I signature score range in the population as the reference IFN-I signature score.

Such reference IFN-I signature score may then be used to distinguish individuals in which therapeutic effectiveness is more likely (IFN-I signature score above the reference IFN-I signature score) from those individuals in which therapeutic effectiveness is less likely (IFN-I activity below the reference IFN-I signature score).

In some embodiments, the reference IFN-I signature score is defined as follows:
(i) Defining a population of patients having the same disease as the individual whose IFN-I signature score is determined according to the methods or uses of the invention and who are treated with the same TLR inhibitor as said individual;
(ii) Determining the IFN-I signature score in a sample from each patient in the patient population before treatment with the TLR inhibitor, wherein the IFN-I signature score is based on the same IFN-I signature as the IFN-I signature on which the IFN-I signature score of the individual whose IFN-I signature score is determined according to the methods or uses of the invention is based on;
(iii) Determining therapeutic effectiveness for each patient in the patient population after treatment with the TLR inhibitor;
(iv) Dividing the patient population into a group that shows more therapeutic effectiveness and a group that shows less therapeutic effectiveness; and
(v) Defining the IFN-I signature score that divides the two patient groups as the reference IFN-I signature score.

Such reference IFN-I signature score may then be used to distinguish individuals in which therapeutic effectiveness is more likely (IFN-I signature score above the reference IFN-I signature score) from those individuals in which therapeutic effectiveness is less likely (IFN-I activity below the reference IFN-I signature score).

In some embodiments, the reference IFN-I signature score is defined as follows:
(xi) Defining a population of patients having the same disease as the individual whose IFN-I signature score is determined according to the methods or uses of the invention and who are treated with the same TLR inhibitor as said individual;
(xii) Determining the IFN-I signature score in a sample from each patient in the patient population before treatment with the TLR inhibitor;
(xiii) Determining therapeutic effectiveness for each patient in the patient population after treatment with the TLR inhibitor;
(xiv) Dividing the patient population into a group that shows more therapeutic effectiveness and a group that shows less therapeutic effectiveness; and
(xv) Defining the IFN-I signature score that is characteristic for one or both of the two patient groups as the reference IFN-I signature score.

Such reference IFN-I signature score may then be used to identify those individuals in which therapeutic effectiveness is more likely (if the IFN-I signature score characteristic for the patient group showing more therapeutic effectiveness was chosen as the reference IFN-I signature score) and/or those individuals in which therapeutic effectiveness is less likely (if the IFN-I signature score characteristic for the patient group showing less therapeutic effectiveness was chosen as the reference IFN-I signature score).

Diseases for which the IFN-I activity (and the IFN-I signature expression pattern or IFN-I signature score) may serve as a biomarker include any disease that is caused, mediated and/or propagated by TLR activity, such as TLR7 and/or TLR8 activity. In some embodiments, the disease is an autoimmune disease. In some embodiments, the disease is an idiopathic inflammatory myopathy, such as polymyositis or dermatomyositis, or a lupus disease, such as systemic lupus erythematosus or lupus nephritis. In some aspects, the disease is selected from the group consisting of arthritis, pancreatitis, mixed connective tissue disease, lupus, myositis, antiphospholipid syndrome, systemic onset arthritis, and irritable bowel syndrome. In some embodiments, the disease is selected from the group consisting of rheumatoid arthritis, autoimmune pancreatitis, systemic lupus erythematosus, cutaneous lupus erythematosus, lupus nephritis, type I diabetes mellitus, multiple sclerosis, antiphospholipid syndrome, sclerosing cholangitis, systemic onset arthritis, irritable bowel disease, scleroderma, Sjogren's disease, vitiligo, polymyositis, dermatomyositis, pemphigus vulgaris, pemphigus foliaceus, inflammatory bowel disease including Crohn's disease and ulcerative colitis, autoimmune hepatitis, hypopituitarism, graft-versus-host disease, autoimmune skin diseases, uveitis, pernicious anemia, and hypoparathyroidism. In some embodiments, the disease is selected from the group consisting of polyangiitis overlap syndrome, Kawasaki's disease, sarcoidosis, glomerulonephritis, and cryopathies. In other aspects, the disease is selected from the group consisting of systemic lupus erythematosus, rheumatoid arthritis, autoimmune skin disease, and multiple sclerosis. In other aspects, the disease is selected from the group consisting of pancreatitis, glomerulonephritis, pyelitis, sclerosing cholangitis, and type I diabetes. In some aspects, the disease is diabetes and/or diabetic-related disease or disorder. In some embodiments, the disease is an inflammatory disease. In some variations, the disease is associated with chronic pathogen stimulation. In some variations, the disease is a viral disease, e.g., resulting from infection with HIV or SARS-COV-2, such as COVID-19. In some embodiments, the disease is selected from rheumatoid arthritis, psoriatic arthritis, osteoarthritis, systemic lupus erythematosus, lupus nephritis, ankylosing spondylitis, osteoporosis, systemic sclerosis, multiple sclerosis, polymyositis, dermatomyositis, psoriasis, type I diabetes, type II diabetes, inflammatory bowel disease, Crohn's disease, ulcerative colitis, hyperimmunoglobulinemia D, periodic fever syndrome, cryopyrin-associated periodic syndromes, Schnitzler's syndrome, systemic juvenile idiopathic arthritis, adult-onset Still's disease, gout, pseudogout, SAPHO syndrome, Castleman's disease, sepsis, stroke, atherosclerosis, celiac disease, DIRA, Alzheimer's disease, Parkinson's disease, and cancer.

TLR inhibitors

In some embodiments, the uses and methods of the invention involve the administration of a TLR inhibitor. In one embodiment, the TLR inhibitor is a TLR7 and/or TLR8 inhibitor. In one embodiment, the TLR inhibitor is a TLR7 and TLR8 inhibitor. In one embodiment, the TLR inhibitor is a small molecule, such as a small molecule inhibitor of TLR7 and/or TLR8.

In one embodiment, the TLR inhibitor is selected from the group consisting of 5-[(3R,5S)-3-amino-5-(trifluoromethyl) piperidin-1-yl]quinoline-8-carbonitrile; (3R,5S)-1-(8-methoxy-1,7-naphthyridin-5-yl)-5-methylpiperidin-3-amine; 2-{4-[2-(7,8-dimethyl[1,2,4]triazolo[1,5-a]pyridin-6-yl)-3-(propan-2-yl)-1H-indol-5-yl]piperidin-1-yl}acetamide; rel-(2R,6R)-4-(8-cyanoquinolin-5yl)-N-((3R,4S)-4-fluoropyrrolidin-3-yl)-6-methylmorpholine-2-carboxamide hydrochloride; (S)—N-(4-((5-(1,6-dimethyl-1H-pyrazolo[3,4-b] pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide; and (R)—N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide or a pharmaceutically acceptable salt of any of these compounds.

In some embodiments, the TLR7 and/or TLR8 inhibitor is a quinoline derivative.

In some embodiments, the TLR7 and/or TLR8 inhibitor is a compound of formula I,

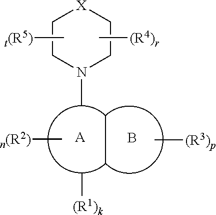

or a pharmaceutically acceptable salt thereof, wherein:
Ring A is aryl or heteroaryl having 1-4 heteroatoms independently selected from nitrogen, oxygen, or sulfur; each of which is optionally substituted;
Ring B is aryl or heteroaryl having 1-4 heteroatoms independently selected from nitrogen, oxygen, or sulfur; each of which is optionally substituted;
R¹ is -Me, —CF₃, —OMe, —OEt, or —CN;
each R² is independently —R, halogen, -haloalkyl, OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R³ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
X is C(R⁴)₂, O, NR₄, S, S(R⁴), or S(R⁴)₂;
each R⁴ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R⁵ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂;
each R is independently hydrogen, C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted; or
two R groups on the same atom are taken together with the atom to which they are attached to form a C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted;
k is 0 or 1;
n is 0, 1, or 2;
p is 0, 1, or 2;
r is 0, 1, or 2; and
t is 0, 1, or 2.

In certain embodiments, Ring A is C₆ aryl or a 6 membered monocyclic heteroaryl having 1-4 heteroatoms independently selected from nitrogen, oxygen, or sulfur; each of which is optionally substituted.

In certain embodiments, Ring A is phenyl, pyridyl, pyrimidinyl, pyrazinyl, pyridazinyl, or triazinyl; each of which is optionally substituted.

In certain embodiments, Ring A is phenyl, pyridyl, or pyrimidinyl; each of which is optionally substituted.

In certain embodiments, Ring B is C₆ aryl or a 5-6 membered monocyclic heteroaryl having 1-4 heteroatoms independently selected from nitrogen, oxygen, or sulfur; each of which is optionally substituted.

In certain embodiments, Ring B is phenyl, pyridyl, pyrimidinyl, pyrazinyl, pyridazinyl, triazinyl, pyrrole, imidazole, isoxazole, oxazole, or thiazole; each of which is optionally substituted.

In certain embodiments, Ring A and Ring B is

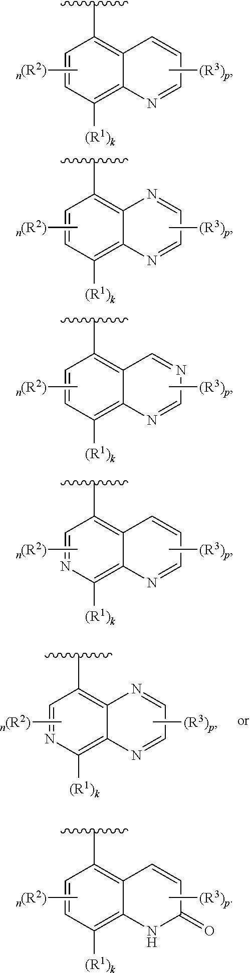

In certain embodiments, Ring A and Ring B is

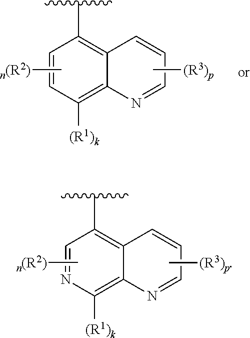

In certain embodiments, Ring A and Ring B is

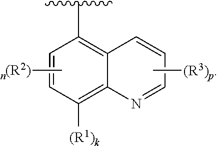

In certain embodiments, Ring A and Ring B is

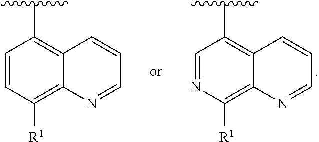

In certain embodiments, Ring A and Ring B is

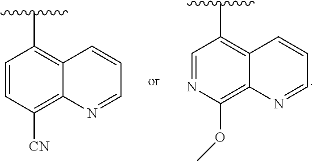 [SMILES: *c1ccc(C#N)c2ncccc12 ; *c1cnc(OC)c2ncccc12]

In certain embodiments, R¹ is —OMe or —CN.

In certain embodiments, each R² is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R² is independently methyl, ethyl, ethyl, propyl, i-propyl, butyl, s-butyl, t-butyl, straight or branched pentyl, or straight or branched hexyl; each of which is optionally substituted.

In certain embodiments, each R² is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R² is independently halogen, -haloalkyl, —OR, —SR, -CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R² is independently-F.

In certain embodiments, each R³ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R³ is independently methyl, ethyl, ethyl, propyl, i-propyl, butyl, s-butyl, t-butyl, straight or branched pentyl, or straight or branched hexyl; each of which is optionally substituted.

In certain embodiments, each R³ is independently methyl.

In certain embodiments, each R³ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R³ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R³ is independently —F.

In certain embodiments, X is C(R⁴)₂ or O.

In certain embodiments, X is C(R⁴)₂. In certain embodiments, X is CH₂.

In certain embodiments, X is O.

In certain embodiments, each R⁴ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁴ is independently methyl, ethyl, ethyl, propyl, i-propyl, butyl, s-butyl, t-butyl, straight or branched pentyl, or straight or branched hexyl; each of which is optionally substituted.

In certain embodiments, each R⁴ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b] tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, 1 pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁴ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or —N(R)₂.

In certain embodiments, each R⁴ is independently-H, C₁₋₆ aliphatic, -OR, —C(O)R, —CO₂R, —C(O)N(R)₂, —NRC(O)R, —NRC(O)N(R)₂, —NRSO₂R, or —N(R)₂; each of which is optionally substituted.

In certain embodiments, each R⁴ is independently —H, C₁₋₆ aliphatic, —C(O)N(R)₂, —NRC(O)R, or —N(R)₂; each of which is optionally substituted.

In certain embodiments, each R⁴ is independently

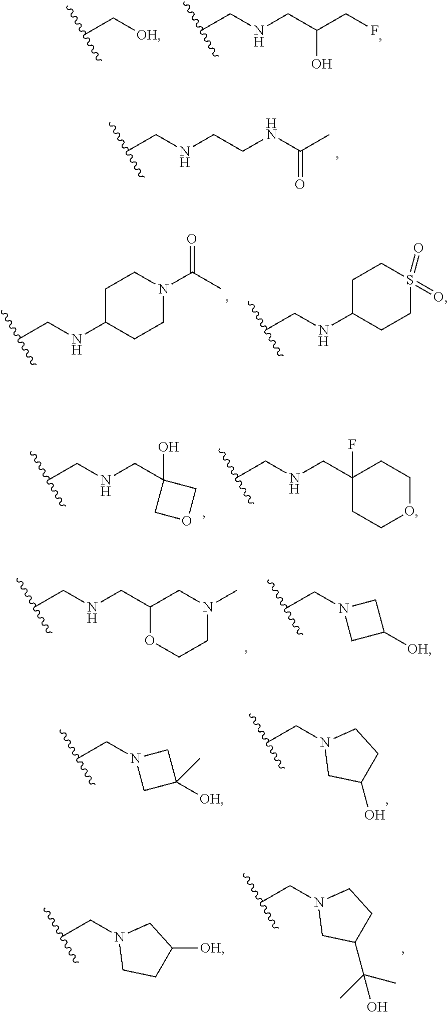

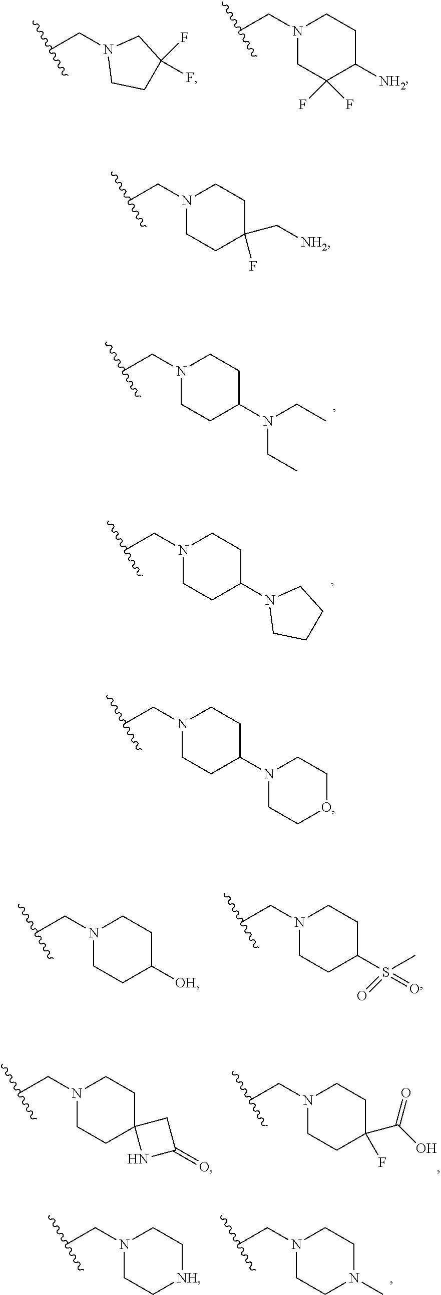

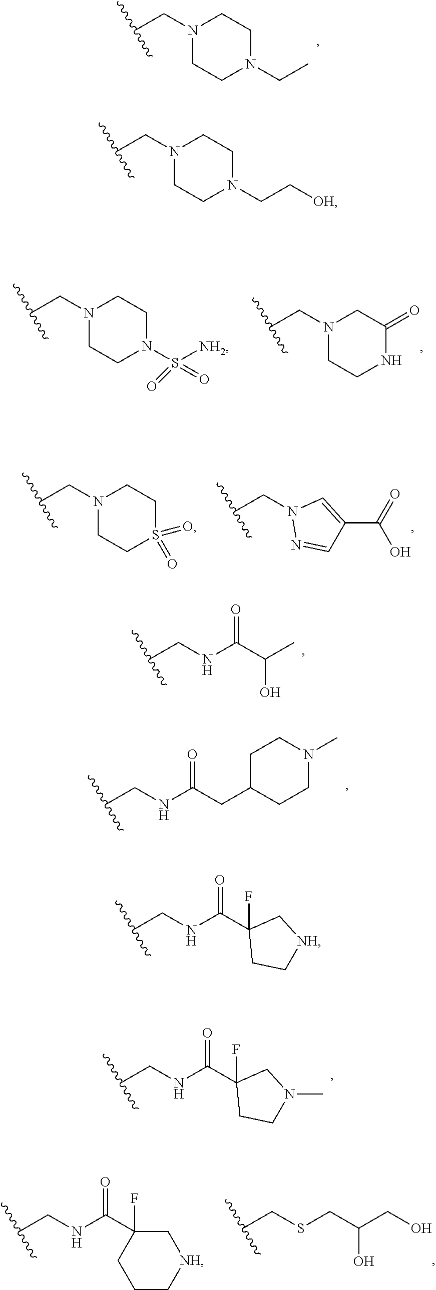

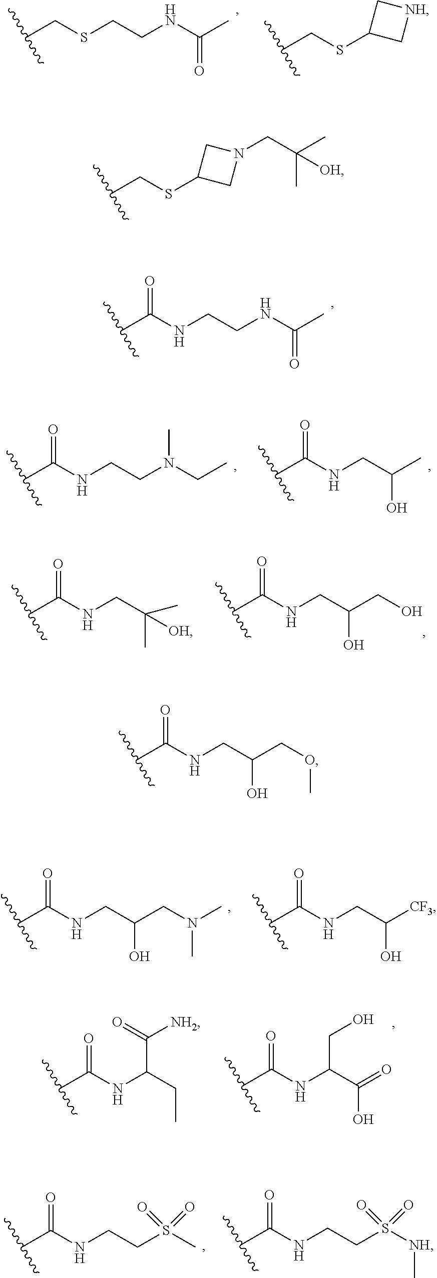

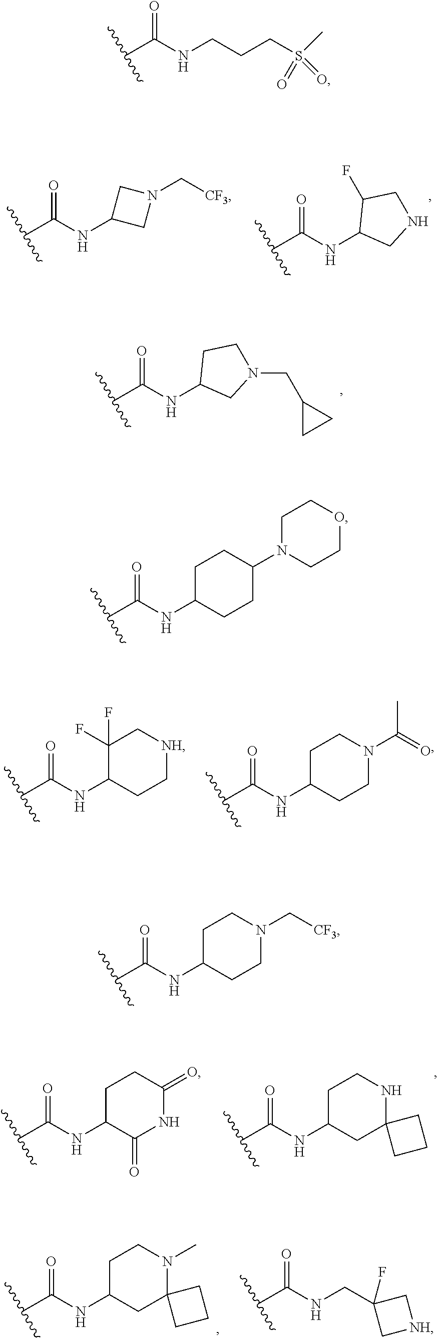

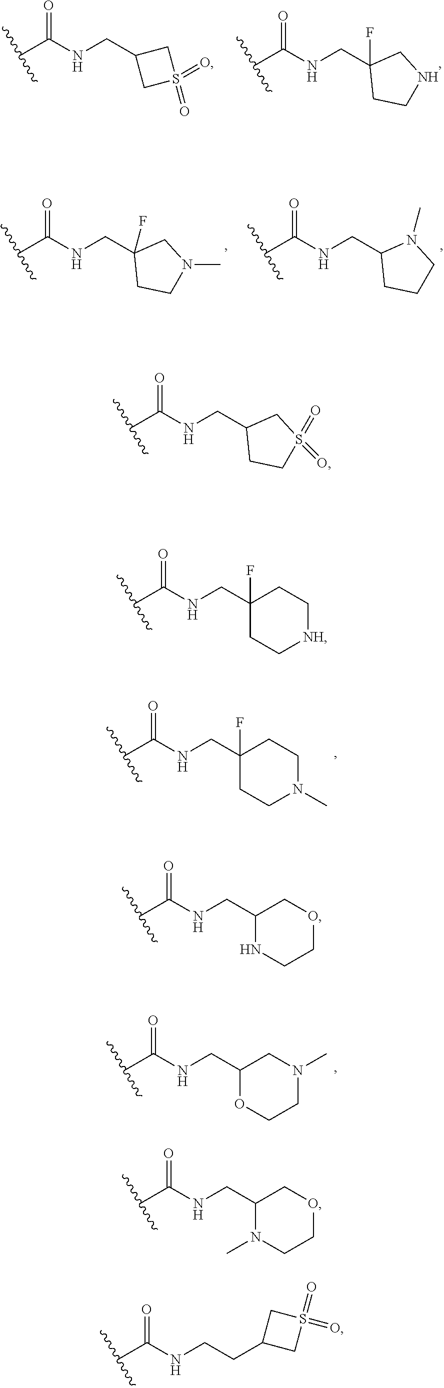

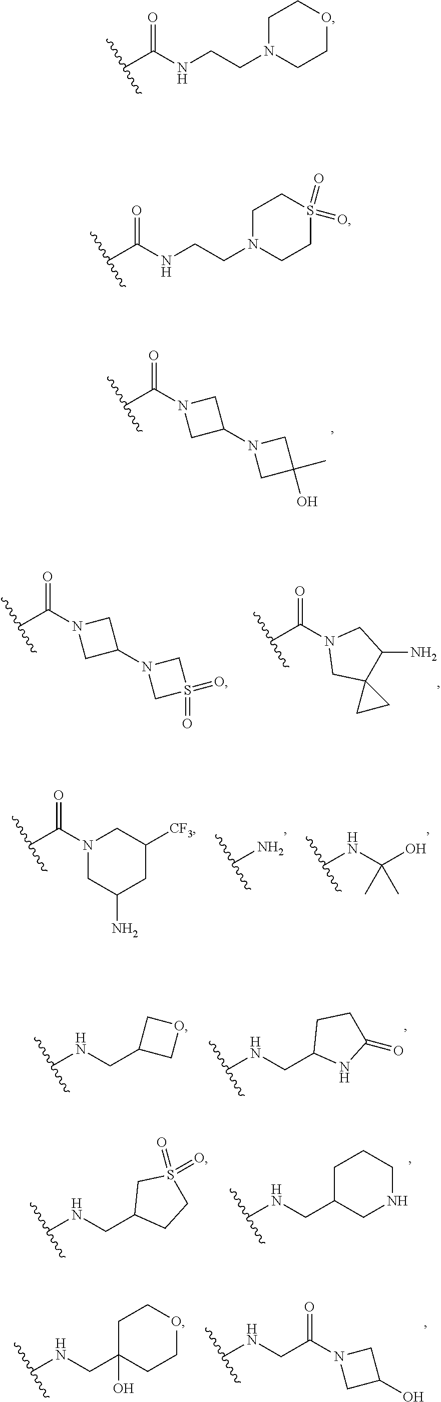

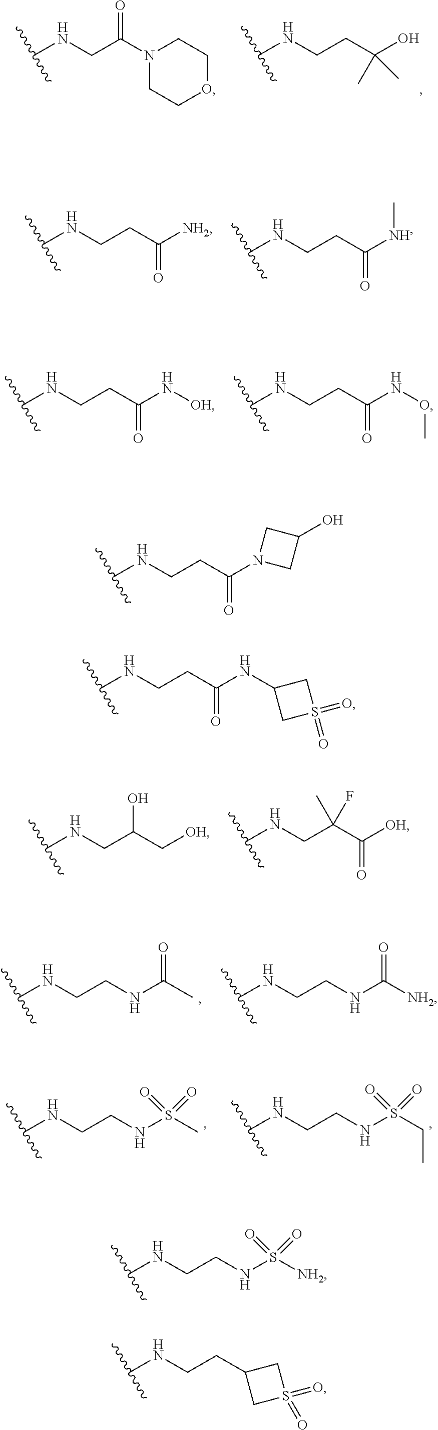

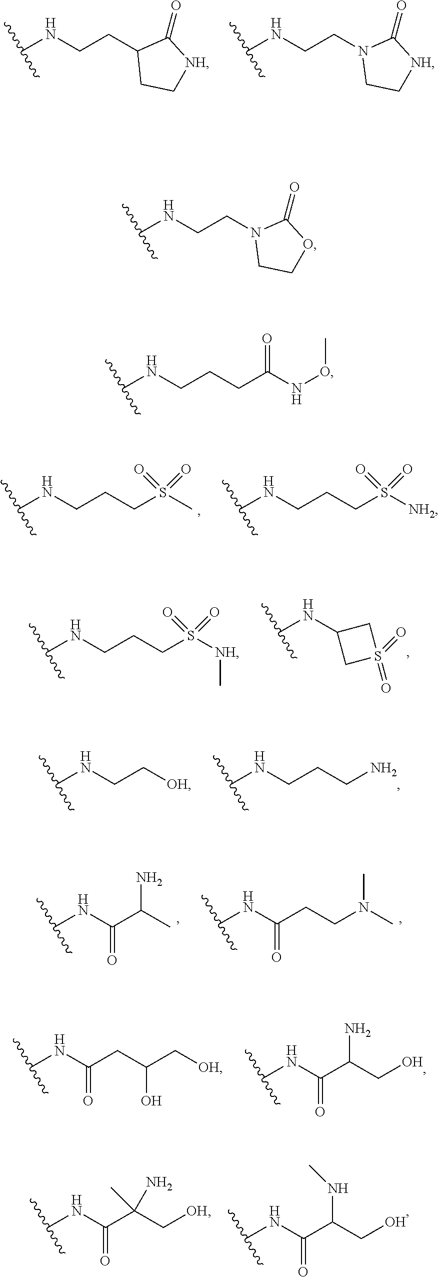

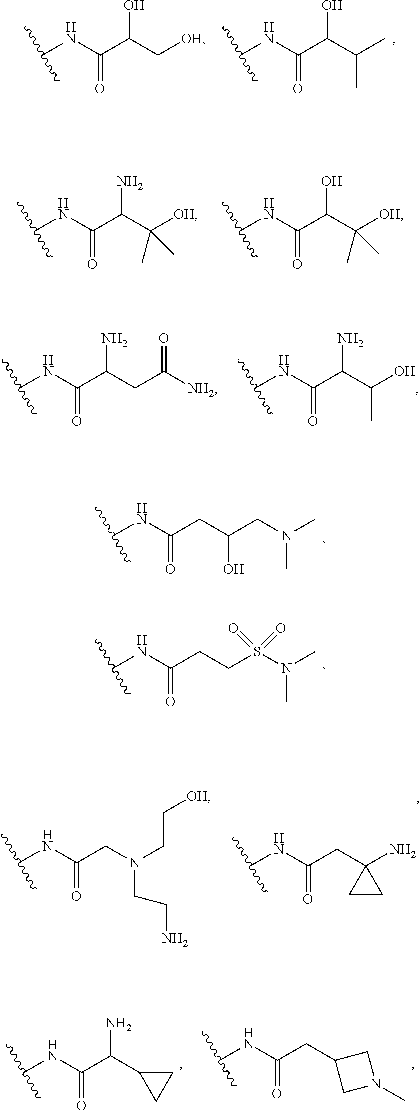

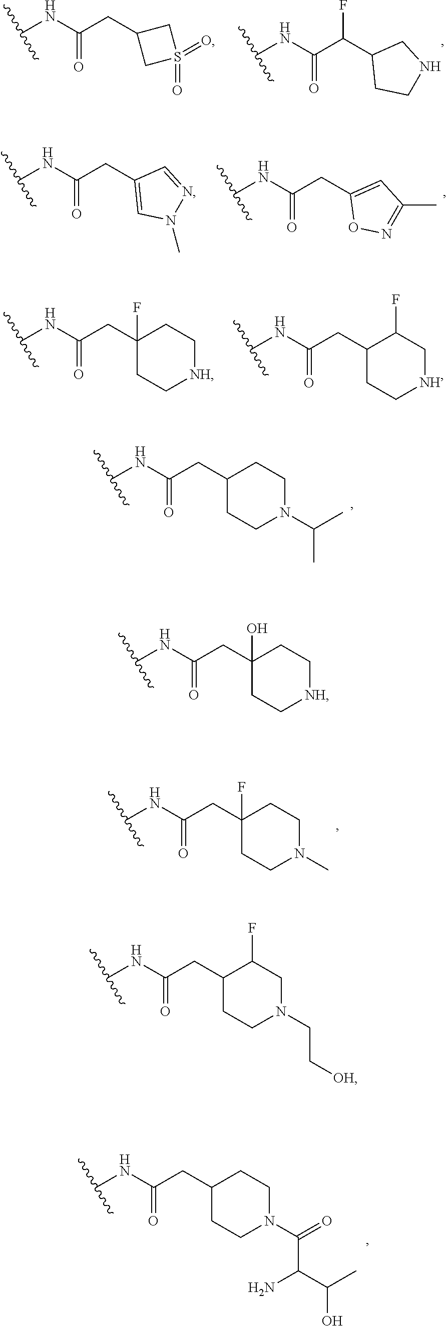

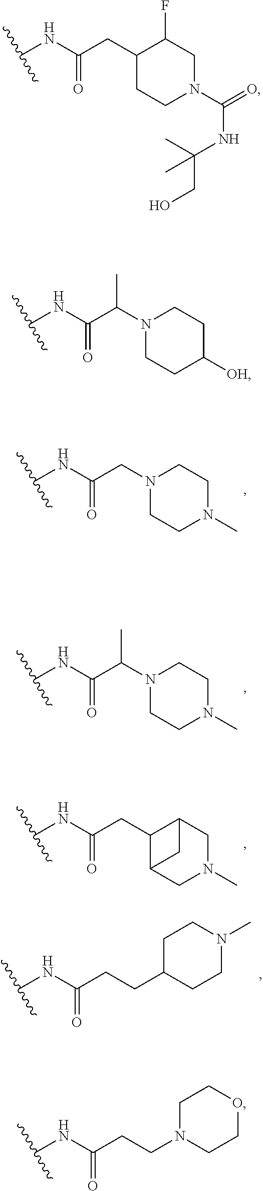

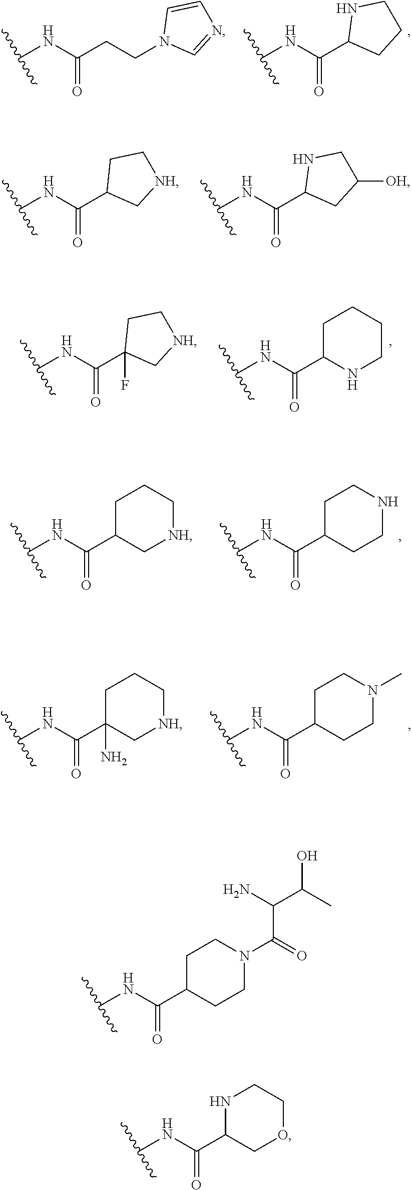

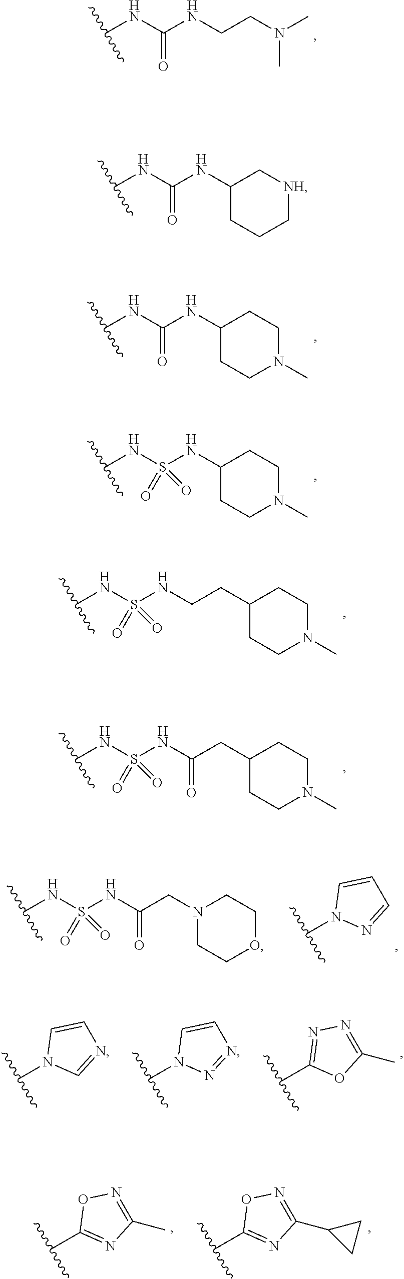

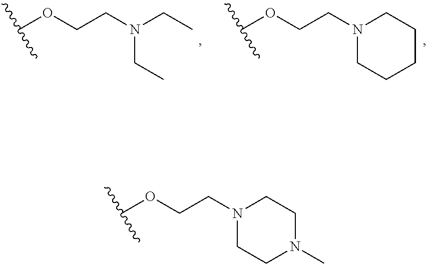

In certain embodiments, each R⁴ is independently

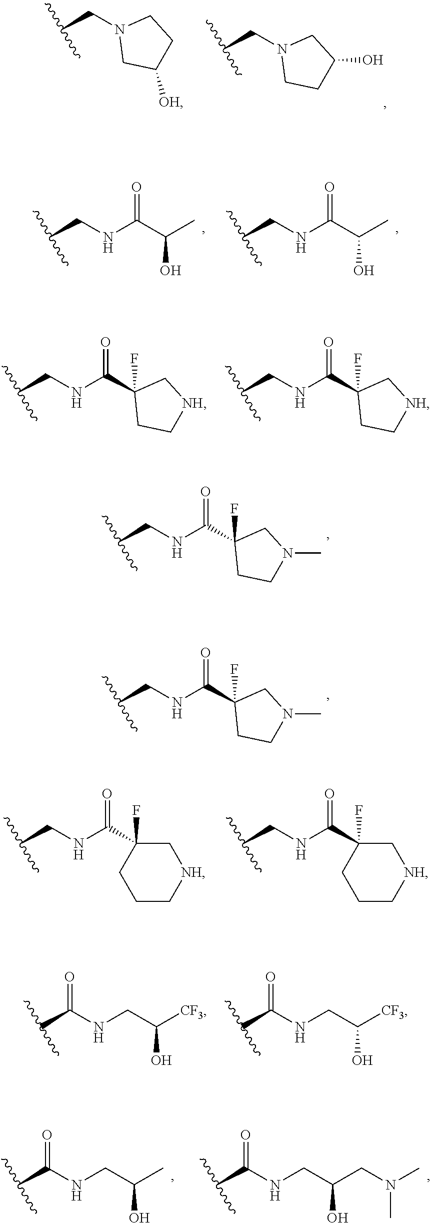

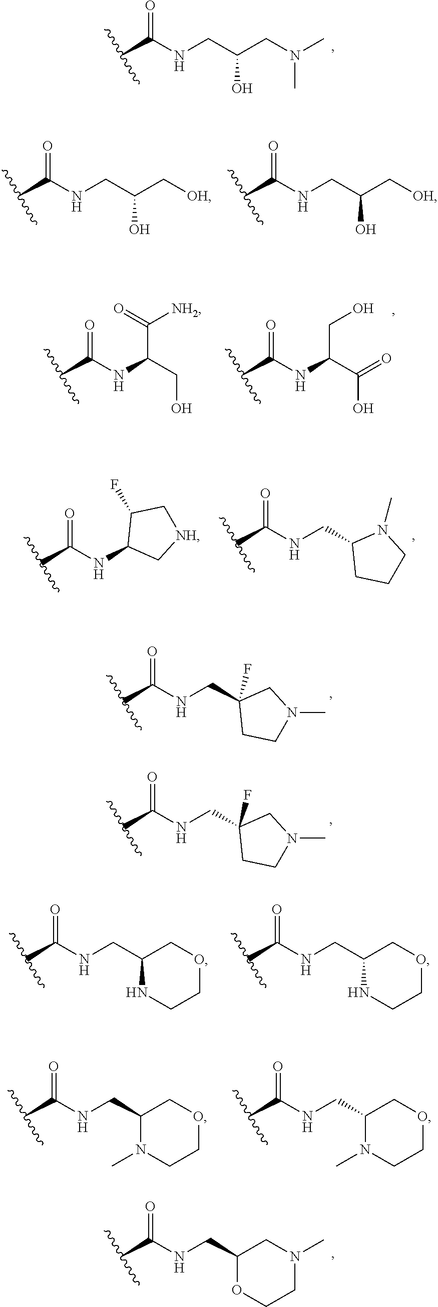

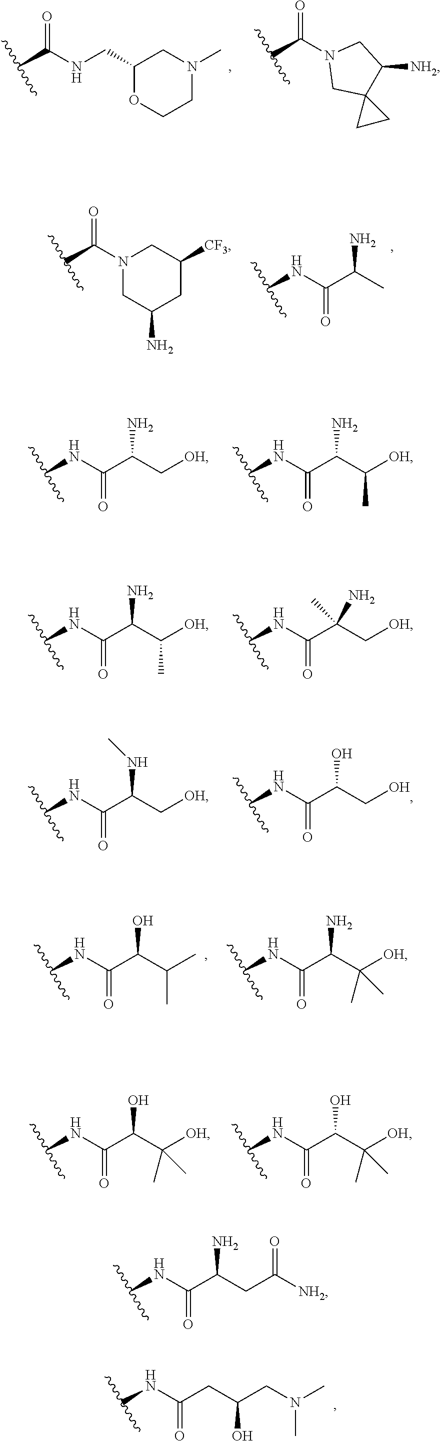

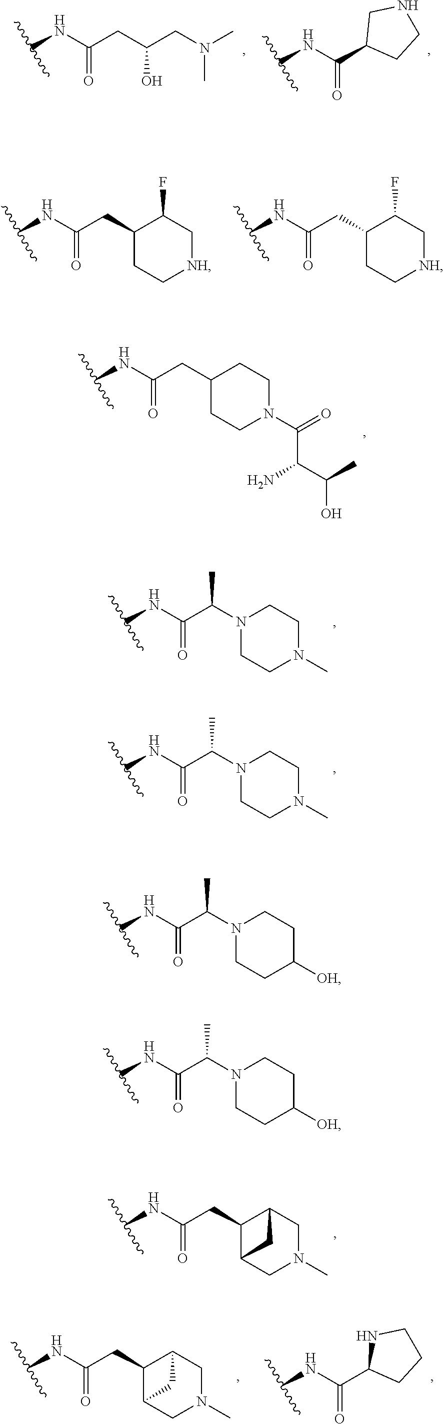

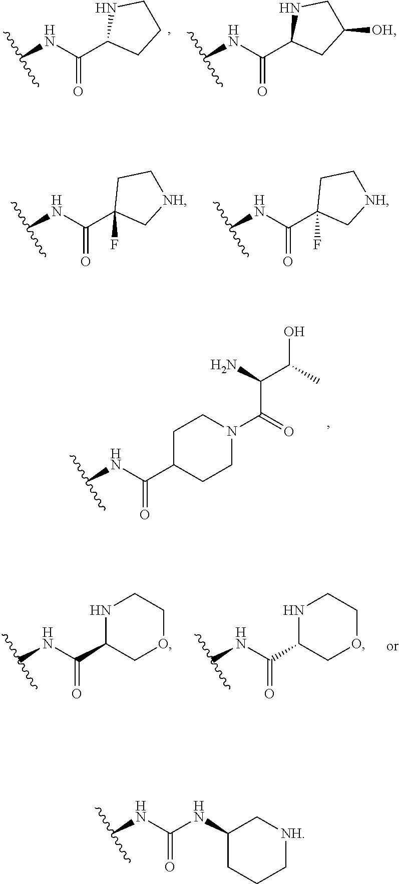

In certain embodiments, each R⁴ is independently

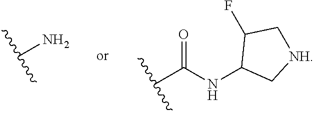

In certain embodiments, each R⁵ is independently C₁₋₆ aliphatic, C₃₋₁₀ aryl, a 3-8 membered saturated or partially unsaturated carbocyclic ring, a 3-7 membered heterocylic ring having 1-4 heteroatoms independently selected from nitrogen, oxygen, or sulfur, or a 5-6 membered monocyclic heteroaryl ring having 1-4 heteroatoms independently selected from nitrogen, oxygen, or sulfur; each of which is optionally substituted.

In certain embodiments, each R⁵ is independently methyl, ethyl, ethyl, propyl, i-propyl, butyl, s-butyl, t-butyl, straight or branched pentyl, or straight or branched hexyl; each of which is optionally substituted.

In certain embodiments, each R⁵ is independently phenyl, naphthyl, cyclopropyl, cyclobutyl, cyclopentyl, cyclohexyl, cycloheptyl, adamantyl, cyclooctyl, [3.3.0]bicyclooctanyl, [4.3.0]bicyclononanyl, [4.4.0]bicyclodecanyl, [2.2.2]bicyclooctanyl, fluorenyl, indanyl, tetrahydronaphthyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, NH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isoindolinyl, isoindolenyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl; -1,2,5oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, oxetanyl, azetidinyl, or xanthenyl; each of which is optionally substituted.

In certain embodiments, each R⁵ is independently halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or -N(R)₂.

In certain embodiments, each R⁵ is independently methyl, cyclopropyl, —F, or —CF₃.

In certain embodiments, each R⁵ is independently

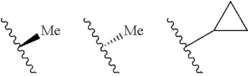

### —F, or —CF₃.

In certain embodiments, k=1. In certain embodiments, r=1. In certain embodiments, t=1. In certain embodiments, n=0. In certain embodiments, p=0. In certain embodiments, both n=0 and p=0. In certain embodiments, r=1 and t=1. In certain embodiments, r=1 and t=1 and k=1. In certain embodiments, r=1 and t=1 and k=1 and n=0 and p=0.

In certain embodiments, each of X, Ring A, Ring B, R¹, R², R³, R₄, R⁵, k, m, n, p, r, and t, is as defined above and described in embodiments, classes and subclasses above and herein, singly or in combination.

In certain embodiments, the TLR7 and/or TLR8 inhibitor is a compound of formula I-a.

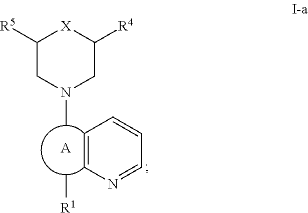

or a pharmaceutically acceptable salt thereof, wherein R¹ is —OMe or —CN, X is O or CH₂, R⁴ is

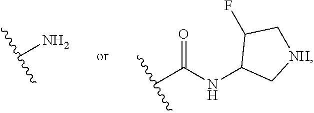

R⁵ is methyl or —CF₃ and Ring A is

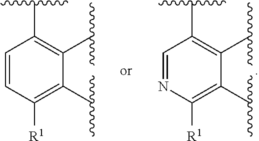

In one embodiment, the TLR inhibitor is a TLR7 and/or TLR8 inhibitor selected from the group consisting of:

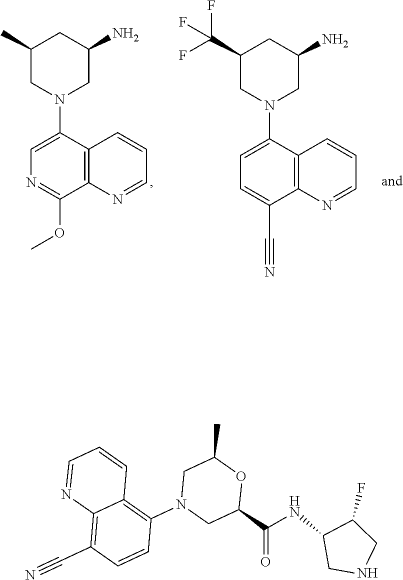 [SMILES: COc1ncc(N2C[C@@H](C)C[C@@H](N)C2)c2cccnc12 ; N#Cc1ccc(N2C[C@H](N)C[C@H](C(F)(F)F)C2)c2cccnc12 ; C[C@@H]1CN(c2ccc(C#N)c3ncccc23)C[C@H](C(=O)N[C@H]2CNC[C@H]2F)O1]

or a pharmaceutically acceptable salt of either of these.

In certain embodiments, the TLR7 and/or TLR8 inhibitor is a compound of formula I-b,

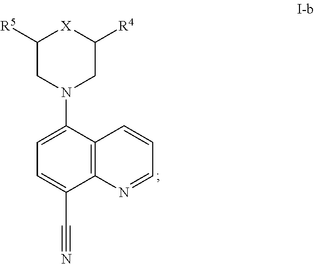

or a pharmaceutically acceptable salt thereof, wherein X is O or CH₂, R⁴ is

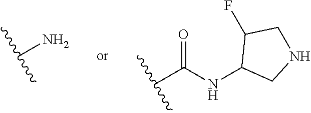

and R⁵ is methyl or —CF₃.

In one embodiment, the TLR inhibitor is a TLR7 and/or TLR8 inhibitor selected from

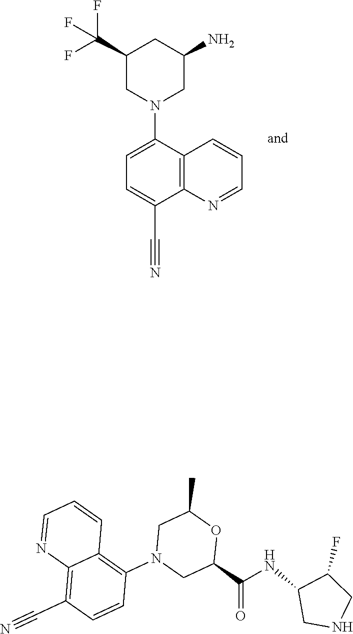 [SMILES: N#Cc1ccc(N2C[C@H](N)C[C@H](C(F)(F)F)C2)c2cccnc12 ; C[C@@H]1CN(c2ccc(C#N)c3ncccc23)C[C@H](C(=O)N[C@H]2CNC[C@H]2F)O1]

or a pharmaceutically acceptable salt of either of these.

In one embodiment, the TLR inhibitor is enpatoran, E6742 or afimetoran.

### Interferon Signature

The choice of the IFN-I signature is not particularly limited, in particular since the IFN-I signatures tend to correlate well.

In one embodiment, the IFN-I signature comprises one or more genes selected from the group consisting of BST2, CMPK2, CXCL10, EPSTII, GBP5, HERC5, HERC6, IFI6, IFI27, IFI44, IFI44L, IFIH1, IFIT1, IFIT2, IFIT3, IRF7, ISG15, LY6E, MX1, MX2, OAS1, OAS2, OAS3, OASL, PKR, RSAD2, SIGLEC1, STAT1, TNFSF10 and USP18, one or more genes selected from the group consisting of BST2, CMPK2, CXCL10, GBP5, HERC6, IFI44, IFIH1, IFIT1, IFIT2, IFIT3, IRF7, ISG15, MX1, MX2, OAS1, OAS2, OAS3, OASL, RSAD2, STATI, TNFSF10 and USP18, one or more genes selected from the group consisting of CMPK2, CXCL10, EPSTI1, HERC5, IFI27, IFI44, IFI44L, IFI6, IFIT1, IFIT3, ISG15, LY6E, MX1, OAS1, OAS2, OAS3, OASL, RSAD2, SIGLEC1 and USP18, one or more genes selected from the group consisting of HERC5, IFI27, IFIT1 and RSAD2, one or more genes selected from the group consisting of ISG15, MX1 and OAS1, one or more genes selected from the group consisting of EPSTI1, HERC5, IFI44L, ISG15, LY6E, MX1, MX2 and RSAD2, one or more genes selected from the group consisting of IFIT1, MX1 and PKR, one or more genes selected from the group consisting of CMPK2, EPSTI and HERC5, one or more genes selected from the group consisting of IFI6, IFI27, IFI44, IFI44L and RSAD2 or one or more genes selected from the group consisting of IFI27, IFI44, IFI44L and RSAD2. In one embodiment, the IFN-I signature comprises at least two, at least three or all genes of one of the groups of genes of the preceding sentence.

In some embodiments, an IFN-I signature score that is higher than a reference IFN-I signature score is at least 10%, 20%, 30%, 40%, 50%, 60%, 70%, 80%, 90%, 100%, 200%, 500% or 1000% higher than the reference IFN-I signature score. In some embodiments, an IFN-I signature score that is lower than a reference IFN-I signature score is at least 10%, 20%, 30%, 40%, 50%, 60%, 70%, 80%, 90% or 95%, lower than the reference IFN-I signature score.

In some embodiments, the IFN-I activity is higher than a reference IFN-I activity is at least 10%, 20%, 30%, 40%, 50%, 60%, 70%, 80%, 90%, 100%, 200%, 500% or 1000% higher than the reference IFN-I activity. In some embodiments, the IFN-I activity that is lower than a reference IFN-I activity is at least 10%, 20%, 30%, 40%, 50%, 60%, 70%, 80%, 90% or 95%, lower than the reference IFN-I activity.

### Formulations and Administration

The TLR inhibitor and other therapeutic agents disclosed herein are administered as such or in a pharmaceutically acceptable composition. They may be administered orally, parenterally, by inhalation spray, topically, rectally, nasally, buccally, vaginally or via an implanted reservoir. In one embodiment, the TLR inhibitor is a small molecule and administered orally. In one embodiment, the oral formulation is a tablet or capsule. In another embodiment, the oral formulation is a solution or suspension which may be given to a subject in need thereof via mouth or nasogastric tube. Any oral formulations of the invention may be administered with or without food. In some embodiments, pharmaceutically acceptable compositions of this invention are administered without food. In other embodiments, pharmaceutically acceptable compositions of this invention are administered with food.

Pharmaceutically acceptable compositions of this invention are orally administered in any orally acceptable dosage form. Exemplary oral dosage forms are capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents are optionally also added.

The amount of compounds of the present invention that are optionally combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

In one embodiment, the total amount of TLR inhibitor administered to the subject in need thereof is between about 10 mg to about 500 mg per day. In one aspect of this embodiment, the total amount of TLR inhibitor administered is between about 50 mg and about 300 mg per day. In another aspect, the total amount of TLR inhibitor administered is between about 100 mg and about 200 mg per day.

In another embodiment, the TLR inhibitor is administered once a day. In another aspect of this embodiment, the TLR inhibitor is administered twice a day.

In one embodiment, the amount of TLR inhibitor administered to the subject in need thereof is about 50 mg twice a day. In another embodiment, the amount of TLR inhibitor administered to the subject in need thereof is about 100 mg twice a day. In another embodiment, the amount of TLR inhibitor administered to the subject in need thereof is about 200 mg twice a day.

In any of the above embodiments, the TLR inhibitor is administered for a period of about 7 day to about 21 days. In one aspect of any of the above embodiments, the TLR inhibitor is administered for about 14 days. In one embodiment, the TLR inhibitor is administered for longer periods of time, e.g., for months or years. In one embodiment, the TLR inhibitor is administered for an indefinite period of time.

In one embodiment of the invention, 50 mg of the TLR inhibitor of the invention is administered twice a day for about 14 days. In one embodiment of the invention, 50 mg of the TLR inhibitor of the invention is administered twice a day for an indefinite period of time. In another embodiment of the invention, 100 mg of the TLR inhibitor of the invention is administered twice a day for about 14 days. In one embodiment of the invention, 100 mg of the TLR inhibitor of the invention is administered twice a day for an indefinite period of time. In another embodiment of the invention, 200 mg of the TLR inhibitor of the invention is administered twice a day for about 14 days. In one embodiment of the invention, 200 mg of the TLR inhibitor of the invention is administered twice a day for an indefinite period of time.

### Combination Treatments

In any of the methods of treatment disclosed herein, the TLR inhibitor can be administered in combination with other known therapeutic agents.

In one aspect of this embodiment, the one or more additional therapeutic agents is selected from anti-inflammatoires, antibiotics, anti-coagulants, antiparasitic agent, antiplatelet agents and dual antiplatelet therapy, angiotensin converting enzyme (ACE) inhibitors, angiotensin II receptor blockers, beta-blockers, statins and other combination cholesterol lowering agents, specific cytokine inhibitors, complement inhibitors, anti-VEGF treatments, JAK inhibitors, immunomodulators, anti-inflammasone therapies, sphingosine-1 phosphate receptors binders, N-methyl-d-aspartate (NDMA) receptor glutamate receptor antagonists, corticosteroids, Granulocyte-macrophage colony-stimulating factor (GM-CSF), anti-GM-CSF, interferons, angiotensin receptor -neprilysin inhibitors, calcium channel blockers, vasodilators, diuretics, muscle relaxants, and antiviral medications.

In one embodiment, the TLR inhibitor is administered in combination with an antiviral agent. In one aspect of this embodiment, the antiviral agent is remdesivir. In another aspect of this embodiment, the antiviral agent is lopinavir-ritonavir, alone or in combination with ribavirin and interferon-beta.

In one embodiment, the TLR inhibitor is administrated in combination with a broad-spectrum antibiotic.

The present inventors surprisingly found increased potency of glucocorticosteroids when combined with TLR7 and/or 8 inhibitors in the context of IFN-α pre-treatment, despite previous studies showing that glucocorticosteroid therapy is less potent in patients with SLE who had a high IFN-I signature score. This suggests that treatment with a TLR inhibitor may even have a glucocorticosteroid-sparing effect in the clinical setting of patients with high IFN-I activitiy. Accordingly, in some embodiments, a TLR inhibitor as described herein is administered in combination with a corticosteroid. In some embodiments, the corticosteroid is a glucocorticosteroid. In some embodiments, the corticosteroid is a mineralocorticoid. Corticosteroids include, but are not limited to, corticosterone and derivatives, prodrugs, isomers and analogs thereof, cortisone and derivatives, prodrugs, isomers and analogs thereof (i.e., Cortone), aldosterone and derivatives, prodrugs, isomers and analogs thereof, dexamethasone and derivatives, prodrugs, isomers and analogs thereof (i.e., Decadron), prednisone and derivatives, prodrugs, isomers and analogs thereof (i.e., Prelone), fludrocortisones and derivatives, prodrugs, isomers and analogs thereof, hydrocortisone and derivatives, prodrugs, isomers and analogs thereof (i.e., cortisol or Cortef), hydroxycortisone and derivatives, prodrugs, isomers and analogs thereof, betamethasone and derivatives, prodrugs, isomers and analogs thereof (i.e., Celestone), budesonide and derivatives, prodrugs, isomers and analogs thereof (i.e., Entocort EC), methylprednisolone and derivatives, prodrugs, isomers and analogs thereof (i.e., Medrol), prednisolone and derivatives, prodrugs, isomers and analogs thereof (i.e., Deltasone, Crtan, Meticorten, Orasone, or Sterapred), triamcinolone and derivatives, prodrugs, isomers and analogs thereof (i.e., Kenacort or Kenalog), and the like. In some embodiments, the corticosteroid is fludrocortisone or a derivative, prodrug, isomer or analog thereof. In some embodiments, the corticosteroid is fludrocortisone. In some embodiments, the corticosteroid is hydroxycortisone or a derivative, prodrug, isomer or analog thereof. In some embodiments, the corticosteroid is hydroxycortisone.

In one embodiment, the TLR inhibitor is administered in combination with chloroquine or hydroxychloroquine. In one aspect of this embodiment, the TLR inhibitor is further combined with azithromycin.

In one embodiment, the TLR inhibitor is administered in combination with interferon-1-beta (Rebif®).

In one embodiment, the TLR inhibitor is administered in combination with dexamethasone.

In one embodiment, the TLR inhibitor is administered in combination with one or more additional therapeutic agents selected from hydroxychloroquine, chloroquine, ivermectin, tranexamic acid, nafamostat, virazole, ribavirin, lopinavir/ritonavir, favipiravir, arbidol, leronlimab, interferon beta-la, interferon beta-1b, beta-interferon, azithromycin, nitrazoxamide, lovastatin, clazakizumab, adalimumab, etanercept, golimumab, infliximab, sarilumab, tocilizumab, anakinra, emapalumab, pirfenidone, belimumab, rituximab, ocrelizumab, anifrolumab, ravulizumab-cwvz, eculizumab, bevacizumab, heparin, enoxaparin, apremilast, coumadin, baricitinib, ruxolitinib, dapagliflozin, methotrexate, leflunomide, azathioprine, sulfasalazine, mycophenolate mofetil, colchicine, fingolimod, ifenprodil, prednisone, cortisol, dexamethasone, methylprednisolone, melatonin, otilimab, ATR-002, APN-01, camostat mesylate, brilacidin, IFX-1, PAX-1-001, BXT-25, NP-120, intravenous immunoglobulin (IVIG), and solnatide.

In one embodiment, the TLR inhibitor is administered in combination with one or more anti-inflammatory agent. In one aspect of this embodiment, the anti-inflammatory agent is selected from corticosteroids, steroids, COX-2 inhibitors, and non-steroidal anti-inflammatory drugs (NSAID). In one aspect of this embodiment, the anti-inflammatory agent is diclofenac, etodolac, fenoprofen, flurbirprofen, ibuprofen, indomethacin, meclofenamate, mefenamic acid, meloxicam, nabumetone, naproxen, oxaprozin, piroxicam, sulindac, tolmetin, celecoxib, prednisone, hydrocortisone, fludocortisone, bethamethasone, prednisolone, triamcinolone, methylprednisone, dexamethasone, fluticasone, and budesonide (alone or in combination with formoterol, salmeterol, or vilanterol).

In one embodiment, the TLR inhibitor is administered in combination with one or more immune modulators. In one aspect of this embodiment the immune modulator is a calcineurin inhibitor, antimetabolite, or alkylating agent. In another aspect of this embodiment, the immune modulator is selected from azathioprine, mycophenolate mofetil, methotrexate, dapson, cyclosporine, cyclophosphamide, and the like.

In one embodiment, the TLR inhibitor is administered in combination with one or more antibiotics. In one aspect of this embodiment, the antibiotic is a broad-spectrum antibiotic. In another aspect of this embodiment, the antibiotic is a pencillin, anti-straphylococcal penicillin, cephalosporin, aminopenicillin (commonly administered with a betalactamase inhibitor), monobactam, quinoline, aminoglycoside, lincosamide, macrolide, tetracycline, glycopeptide, antimetabolite or nitroimidazole. In a further aspect of this embodiment, the antibiotic is selected from penicillin G, oxacillin, amoxicillin, cefazolin, cephalexin, cephotetan, cefoxitin, ceftriazone, augmentin, amoxicillin, ampicillin (plus sulbactam), piperacillin (plus tazobactam), ertapenem, ciprofloxacin, imipenem, meropenem, levofloxacin, moxifloxacin, amikacin, clindamycin, azithromycin, doxycycline, vancomycin, Bactrim, and metronidazole.

In one embodiment, the TLR inhibitor is administered in combination with one or more anti-coagulants. In one aspect of this embodiment, the anti-coagulant is selected from apixaban, dabigatran, edoxaban, heparin, rivaroxaban, and warfarin.

In one embodiment, the TLR inhibitor is administered in combination with one or more antiplatlet agents and/or dual antiplatelet therapy. In one aspect of this embodiment, the antiplatelet agent and/or dual antiplatelet therapy is selected from aspirin, clopidogrel, dipyridamole, prasugrel, and ticagrelor.

In one embodiment, the TLR inhibitor is administered in combination with one or more ACE inhibitors. In one aspect of this embodiment, the ACE inhibitor is selected from benazepril, captopril, enalapril, fosinopril, lisinopril, moexipril, perindopril, quinapril, ramipril and trandoliapril.

In one embodiment, the TLR inhibitor is administered in combination with one or more angiotensin II receptor blockers. In one aspect of this embodiment, the angiotensin II receptor blocker is selected from azilsartan, candesartan, eprosartan, irbesartan, losartan, Olmesartan, telmisartan, and valsartan.

In one embodiment, the TLR inhibitor is administered in combination with one or more beta-blockers. In one aspect of this embodiment, the beta-blocker is selected from acebutolol, atenolol, betaxolol, bisoprolol/hydrochlorothiazide, bisoprolol, metoprolol, nadolol, propranolol, and sotalol.

In another embodiment, the TLR inhibitor is administered in combination with one or more alpha and beta-blocker. In one aspect of this embodiment, the alpha and beta-blocker is carvedilol or labetalol hydrochloride.

In one embodiment, the TLR inhibitor is administered in combination with one or more interferons.

In one embodiment, the TLR inhibitor is administered in combination with one or more angiotensin receptor -neprilysin inhibitors. In one aspect of this embodiment, the angiotensin receptor -neprilysin inhibitor is is sacubitril/valsartan.

In one embodiment, the TLR inhibitor is administered in combination with one or more calcium channel blockers. In one aspect of this embodiment, the calcium channel blocker is selected from amlodipine, diltiazem, felodipine, nifedipine, nimodipine, nisoldipine, and verapamil.

In one embodiment, the TLR inhibitor is administered in combination with one or more vasodilators. In one aspect of this embodiment, the one or more vasodilator is selected from isosorbide dinitrate, isosorbide mononitrate, nitroglycerin, and minoxidil.

In one embodiment, the TLR inhibitor is administered in combination with one or more diuretics. In one aspect of this embodiment, the one or more diuretics is selected from amiloride, bumetanide, chlorothiazide, chlorthalidone, furosemide, acetazolamide, hydrochlorothiazide, indapamide, metalozone, spironolactone, and torsemide.

In one embodiment, the TLR inhibitor is administered in combination with one or more muscle relaxants. In one aspect of this embodiment, the muscle relaxant is an antispasmodic or antispastic. In another aspect of this embodiment, the one or more muscle relaxants is selected from casisoprodol, chlorzoxazone, cyclobenzaprine, metaxalone, methocarbamol, orphenadrine, tizanidine, baclofen, dantrolene, and diazepam.

In one embodiment, the TLR inhibitor is administered in combination with one or more antiviral medications. In one aspect of this embodiment, the antiviral medication is remdesivir.

In one embodiment, the TLR inhibitor is administered in combination with one or more additional therapeutic agents selected from antiparasitic drugs (including, but not limited to, hydroxychloroquine, chloroquine, ivermectin), antivirals (including, but not limited to, tranexamic acid, nafamostat, virazole [ribavirin], lopinavir/ritonavir, favipiravir, leronlimab, interferon beta-la, interferon beta-1b, beta-interferon), antibiotics with intracellular activities (including, but not limited to azithromycin, nitrazoxamide), statins and other combination cholesterol lowering and anti-inflammatory drugs (including, but not limited to, lovastatin), specific cytokine inhibitors (including, but not limited to, clazakizumab, adalimumab, etanercept, golimumab, infliximab, sarilumab, tocilizumab, anakinra, emapalumab, pirfenidone), complement inhibitors (including, but not limited to, ravulizumab-cwvz, eculizumab), anti-VEGF treatments (including, but not limited to, bevacizumab), anti-coagulants (including, but not limited to, heparin, enoxaparin, apremilast, coumadin), JAK inhibitors (including, but not limited to, baricitinib, ruxolitinib, dapagliflozin), anti-inflammasone therapies (including, but not limited to, colchicine), sphingosine-1 phosphate receptors binders (including, but not limited to, fingolimod), N-methyl-d-aspartate (NDMA) receptor glutamate receptor antagonists (including, but not limited to, ifenprodil), corticosteroids (including, but not limited to, prednisone, cortisol, dexamethasone, methylprednisolone), GM-CSF, anti-GM-CSF (otilimab), ATR-002, APN-01, camostat mesylate, arbidol, brilacidin, IFX-1, PAX-1-001, BXT-25, NP-120, intravenous immunoglobulin (IVIG), and solnatide.

In some embodiments, the combination of a TLR inhibitor with one or more additional therapeutic agents reduces the effective amount (including, but not limited to, dosage volume, dosage concentration, and/or total drug dose administered) of the TLR inhibitor and/or the one or more additional therapeutic agents administered to achieve the same result as compared to the effective amount administered when the TLR inhibitor or the additional therapeutic agent is administered alone. In some embodiments, reduced effective amounts of corticosteroids can be administered when co-administered with a TLR inhibitor, for instance, in an individual having high IFN-I activity. In some embodiments, the combination of a TLR inhibitor with the additional therapeutic agent reduces the total duration of treatment compared to administration of the additional therapeutic agent alone. In some embodiments, the combination of a TLR inhibitor with the additional therapeutic agent reduces the side effects associated with administration of the additional therapeutic agent alone. In some embodiments, the combination of an effective amount of the TLR inhibitor with the additional therapeutic agent is more efficacious compared to an effective amount of the TLR inhibitor or the additional therapeutic agent alone. In one embodiment, the combination of an effective amount of the TLR inhibitor with the one or more additional therapeutic agent results in one or more additional clinical benefits than administration of either agent alone.

### Methods for Advertising

In one aspect, the invention provides a method for advertising a TLR inhibitor comprising promoting, to a target audience, the use of the TLR inhibitor for treating a disease in an individual based on IFN-I activity. In another aspect, the invention provides a method for advertising a TLR inhibitor comprising promoting, to a target audience, the use of the TLR inhibitor for treating a disease in an individual having high IFN-I activity, e.g., in an individual having an IFN-I signature score above a reference IFN-I signature score. Promotion may be conducted by any means available. In some embodiments, the promotion is by a package insert accompanying the TLR inhibitor. The promotion may also be by a package insert accompanying another therapeutic agent, such as the therapeutic agents mentioned herein that the TLR inhibitor can be combined with. In some embodiments, the promotion is by a package insert where the package insert provides instructions to receive therapy with the TLR inhibitor after measuring IFN-I activity, e.g., by determining an IFN-I signature score, and in some embodiments, in combination with another therapeutic agent. In some embodiments, the promotion is followed by the treatment of the patient with the TLR inhibitor with or without another therapeutic agent. In some embodiments, the package insert indicates that the TLR inhibitor is to be used to treat the patient if the patient's sample is characterized by high IFN-I activity, e.g., an IFN-I signature score of the patient above a reference IFN-I signature score. In some embodiments, the package insert indicates that the TLR inhibitor is not to be used to treat the patient if the patient's sample is characterized by low IFN-I activity, e.g., an IFN-I signature score of the patient below a reference IFN-I signature score. In some embodiments, a high IFN-I activity means a measured IFN-I activity that correlates with a likelihood of increased therapeutic effectiveness when the patient is treated with the TLR inhibitor, and vice versa.

### Further Embodiments of the Present Disclosure

E1. A method for predicting therapeutic effectiveness of a TLR inhibitor in an individual with a disease comprising determining the IFN-I activity in a sample from the individual, wherein the IFN-I activity in the sample indicates the therapeutic effectiveness of the TLR inhibitor.

E2. A method for predicting therapeutic effectiveness of a TLR inhibitor in an individual with a disease comprising determining the IFN-I activity in a sample from the individual, wherein the IFN-I activity in the sample indicates the therapeutic effectiveness of the TLR inhibitor and wherein the TLR inhibitor is a TLR7 and/or TLR8 small molecule inhibitor.

E3. A method for predicting therapeutic effectiveness of a TLR inhibitor in an individual with a disease comprising determining the IFN-I activity in a sample from the individual, wherein the IFN-I activity in the sample indicates the therapeutic effectiveness of the TLR inhibitor and wherein the TLR inhibitor is a TLR7 and/or TLR8 inhibitor of formula I-a,

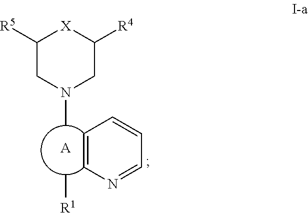

or a pharmaceutically acceptable salt thereof, wherein R¹ is —OMe or —CN, X is O or CH₂, R⁴ is

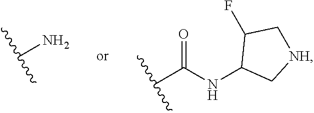

R⁵ is methyl or —CF₃ and Ring A is

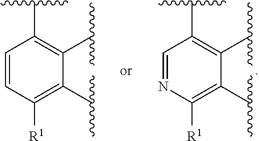

E4. The method according to any one of E1 to E3, wherein the TLR inhibitor is predicted to be therapeutically effective if the individual has high IFN-I activity and the TLR inhibitor is predicted not to be therapeutically effective if the individual has low IFN-I activity.

E5. The method according to any one of E1 to E3, wherein the IFN-I activity is compared to a reference IFN-I activity and the TLR inhibitor is predicted to be therapeutically effective if the IFN-I activity of the individual is higher than the reference IFN-I activity and predicted not to be therapeutically effective if the IFN-I activity of the individual is less than the reference IFN-I activity.

E6. A method for predicting therapeutic effectiveness of a TLR inhibitor in an individual with a disease comprising determining an IFN-I signature expression pattern in a sample from the individual, wherein the IFN-I signature expression pattern in the sample indicates the therapeutic effectiveness of the TLR inhibitor.

E7. A method for predicting therapeutic effectiveness of a TLR inhibitor in an individual with a disease comprising determining an IFN-I signature expression pattern in a sample from the individual, wherein the IFN-I signature expression pattern in the sample indicates the therapeutic effectiveness of the TLR inhibitor and the TLR inhibitor is a TLR7 and/or TLR8 small molecule inhibitor.

E8. A method for predicting therapeutic effectiveness of a TLR inhibitor in an individual with a disease comprising determining an IFN-I signature expression pattern in a sample from the individual, wherein the IFN-I signature expression pattern in the sample indicates the therapeutic effectiveness of the TLR inhibitor and the TLR inhibitor is a TLR7 and/or TLR8 inhibitor of formula I-a,

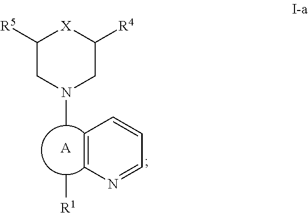

or a pharmaceutically acceptable salt thereof, wherein R¹ is —OMe or —CN, X is O or CH₂, R⁴ is

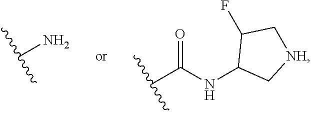

R⁵ is methyl or —CF₃ and Ring A is

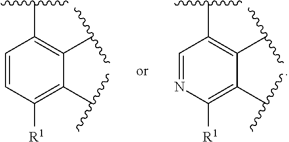

E9. A method for predicting therapeutic effectiveness of a TLR inhibitor in an individual with a disease comprising determining an IFN-I signature score in a sample from the individual, wherein the TLR inhibitor is predicted to be therapeutically effective if the determined IFN-I signature score is higher than a reference IFN-I signature score and predicted not to be therapeutically effective if the determined IFN-I signature score is lower than the reference IFN-I signature score.

E10. A method for predicting therapeutic effectiveness of a TLR inhibitor in an individual with a disease comprising determining an IFN-I signature score in a sample from the individual, wherein the TLR inhibitor is predicted to be therapeutically effective if the determined IFN-I signature score is higher than a reference IFN-I signature score and predicted not to be therapeutically effective if the determined IFN-I signature score is lower than the reference IFN-I signature score and wherein the TLR inhibitor is a TLR7 and/or TLR8 small molecule inhibitor.

E11. A method for predicting therapeutic effectiveness of a TLR inhibitor in an individual with a disease comprising determining an IFN-I signature score in a sample from the individual, wherein the TLR inhibitor is predicted to be therapeutically effective if the determined IFN-I signature score is higher than a reference IFN-I signature score and predicted not to be therapeutically effective if the determined IFN-I signature score is lower than the reference IFN-I signature score and wherein the TLR inhibitor is a TLR7 and/or TLR8 inhibitor of formula I-a,

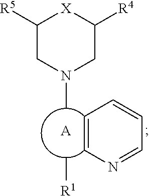

or a pharmaceutically acceptable salt thereof, wherein R¹ is —OMe or —CN, X is O or CH₂, R⁴ is

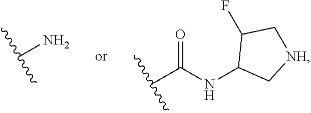

R⁵ is methyl or —CF₃ and Ring A is

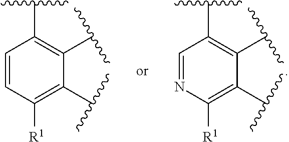

E12. A method for predicting the suitability of an individual with a disease to initiate treatment with a TLR inhibitor comprising determining the IFN-I activity in a sample from the individual, wherein the IFN-I activity in the sample indicates the suitability of the individual to initiate the treatment.

E13. A method for predicting the suitability of an individual with a disease to initiate treatment with a TLR inhibitor comprising determining the IFN-I activity in a sample from the individual, wherein the IFN-I activity in the sample indicates the suitability of the individual to initiate the treatment and wherein the TLR inhibitor is a TLR7 and/or TLR8 small molecule inhibitor.

E14. A method for predicting the suitability of an individual with a disease to initiate treatment with a TLR inhibitor comprising determining the IFN-I activity in a sample from the individual, wherein the IFN-I activity in the sample indicates the suitability of the individual to initiate the treatment and wherein the TLR inhibitor is a TLR7 and/or TLR8 inhibitor of formula I-a,

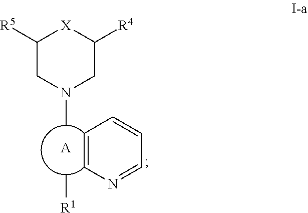

or a pharmaceutically acceptable salt thereof, wherein R¹ is —OMe or —CN, X is O or CH₂, R⁴ is

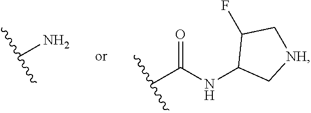

R⁵ is methyl or —CF₃ and Ring A is

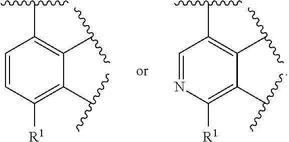

E15. The method according to any one of E12 to E14, wherein an individual having high IFN-I activity is assessed to be suitable for initiating the treatment and an individual having low IFN-I activity is assessed to be unsuitable for initiating the treatment.

E16. The method according to any one of E12 to E14, wherein the IFN-I activity is compared to a reference IFN-I activity and the individual is assessed to be suitable to initiate the treatment if the IFN-I activity of the individual is higher than the reference IFN-I activity and assessed to be unsuitable to initiate the treatment if the IFN-I activity of the individual is lower than the reference IFN-I activity.

E17. A method for predicting the suitability of an individual with a disease to initiate treatment with a TLR inhibitor comprising determining an IFN-I signature expression pattern in a sample from the individual, wherein the IFN-I signature expression pattern in the sample indicates the suitability of the individual to initiate the treatment.

E18. A method for predicting the suitability of an individual with a disease to initiate treatment with a TLR inhibitor comprising determining an IFN-I signature expression pattern in a sample from the individual, wherein the IFN-I signature expression pattern in the sample indicates the suitability of the individual to initiate the treatment and wherein the TLR inhibitor is a TLR7 and/or TLR8 small molecule inhibitor.

E19. A method for predicting the suitability of an individual with a disease to initiate treatment with a TLR inhibitor comprising determining an IFN-I signature expression pattern in a sample from the individual, wherein the IFN-I signature expression pattern in the sample indicates the suitability of the individual to initiate the treatment and wherein the TLR inhibitor is a TLR7 and/or TLR8 inhibitor of formula I-a,

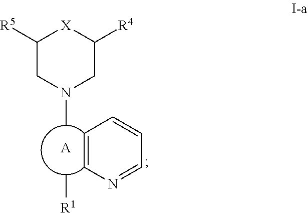

or a pharmaceutically acceptable salt thereof, wherein R¹ is —OMe or —CN, X is O or CH₂, R⁴ is

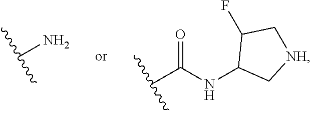

R⁵ is methyl or —CF₃ and Ring A is

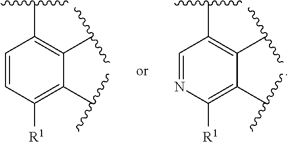

E20. A method for predicting the suitability of an individual with a disease to initiate treatment with a TLR inhibitor comprising determining an IFN-I signature score in a sample from the individual, wherein the individual is assessed to be suitable to initiate the treatment if the determined IFN-I signature score is higher than a reference IFN-I signature score and unsuitable to initiate the treatment if the determined IFN-I signature score is less than the reference IFN-I signature score.

E21. A method for predicting the suitability of an individual with a disease to initiate treatment with a TLR inhibitor comprising determining an IFN-I signature score in a sample from the individual, wherein the individual is assessed to be suitable to initiate the treatment if the determined IFN-I signature score is higher than a reference IFN-I signature score and unsuitable to initiate the treatment if the determined IFN-I signature score is less than the reference IFN-I signature score and wherein the TLR inhibitor is a TLR7 and/or TLR8 small molecule inhibitor.

E22. A method for predicting the suitability of an individual with a disease to initiate treatment with a TLR inhibitor comprising determining an IFN-I signature score in a sample from the individual, wherein the individual is assessed to be suitable to initiate the treatment if the determined IFN-I signature score is higher than a reference IFN-I signature score and unsuitable to initiate the treatment if the determined IFN-I signature score is less than the reference IFN-I signature score and wherein the TLR inhibitor is a TLR7 and/or TLR8 inhibitor of formula I-a,

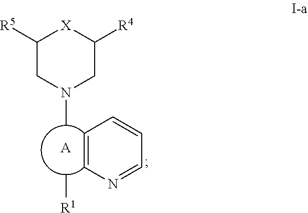

or a pharmaceutically acceptable salt thereof, wherein R¹ is —OMe or —CN, X is O or CH₂, R⁴ is

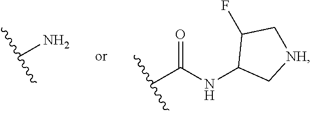

R⁵ is methyl or —CF₃ and Ring A is

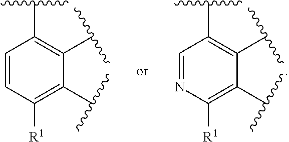

E23. A method for predicting the suitability of an individual with a disease who is receiving treatment with a TLR inhibitor to continue the treatment comprising determining the IFN-I activity in a sample from the individual, wherein the IFN-I activity in the sample indicates the suitability of the individual to continue the treatment.

E24. A method for predicting the suitability of an individual with a disease who is receiving treatment with a TLR inhibitor to continue the treatment comprising determining the IFN-I activity in a sample from the individual, wherein the IFN-I activity in the sample indicates the suitability of the individual to continue the treatment and wherein the TLR inhibitor is a TLR7 and/or TLR8 small molecule inhibitor.

E25. A method for predicting the suitability of an individual with a disease who is receiving treatment with a TLR inhibitor to continue the treatment comprising determining the IFN-I activity in a sample from the individual, wherein the IFN-I activity in the sample indicates the suitability of the individual to continue the treatment and wherein the TLR inhibitor is a TLR7 and/or TLR8 inhibitor of formula I-a,

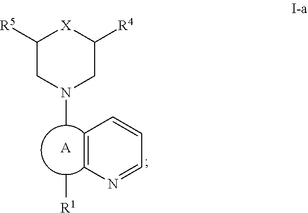

or a pharmaceutically acceptable salt thereof, wherein R¹ is —OMe or —CN, X is O or CH₂, R⁴ is

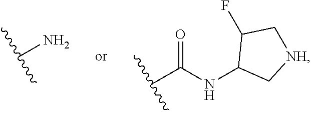

R⁵ is methyl or —CF₃ and Ring A is

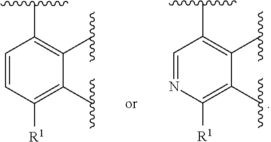

E26. The method according to any one of E23 to E25, wherein an individual having high IFN-I activity is assessed to be suitable to continue the treatment and an individual having low IFN-I activity is assessed to be unsuitable to continue the treatment.

E27. The method according to any one of E23 to E25, wherein the IFN-I activity is compared to a reference IFN-I activity and the individual is assessed to be suitable to continue the treatment if the IFN-I activity of the individual is higher than the reference IFN-I activity and unsuitable to continue the treatment if the IFN-I activity of the individual is lower than the reference IFN-I activity.

E28. A method for predicting the suitability of an individual with a disease who is receiving treatment with a TLR inhibitor to continue the treatment comprising determining an IFN-I signature expression pattern in a sample from the individual, wherein the IFN-I signature expression pattern in the sample indicates the suitability of the individual to continue the treatment.

E29. A method for predicting the suitability of an individual with a disease who is receiving treatment with a TLR inhibitor to continue the treatment comprising determining an IFN-I signature expression pattern in a sample from the individual, wherein the IFN-I signature expression pattern in the sample indicates the suitability of the individual to continue the treatment and wherein the TLR inhibitor is a TLR7 and/or TLR8 small molecule inhibitor.

E30. A method for predicting the suitability of an individual with a disease who is receiving treatment with a TLR inhibitor to continue the treatment comprising determining an IFN-I signature expression pattern in a sample from the individual, wherein the IFN-I signature expression pattern in the sample indicates the suitability of the individual to continue the treatment and wherein the TLR inhibitor is a TLR7 and/or TLR8 inhibitor of formula I-a,

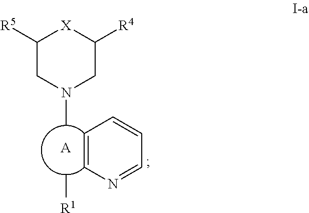

or a pharmaceutically acceptable salt thereof, wherein R¹ is —OMe or —CN, X is O or CH₂, R⁴ is

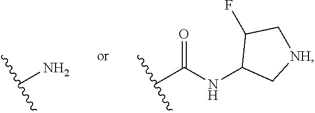

R⁵ is methyl or —CF₃ and Ring A is

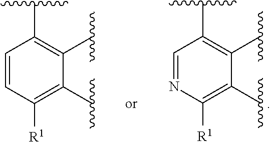

E31. A method for predicting the suitability of an individual with a disease who is receiving treatment with a TLR inhibitor to continue said treatment comprising determining an IFN-I signature score in a sample from the individual, wherein the individual is assessed to be suitable to continue the treatment if the determined IFN-I signature score is higher than a reference IFN-I signature score and unsuitable to continue the treatment if the determined IFN-I signature score is lower than the reference IFN-I signature score.

E32. A method for predicting the suitability of an individual with a disease who is receiving treatment with a TLR inhibitor to continue said treatment comprising determining an IFN-I signature score in a sample from the individual, wherein the individual is assessed to be suitable to continue the treatment if the determined IFN-I signature score is higher than a reference IFN-I signature score and unsuitable to continue the treatment if the determined IFN-I signature score is lower than the reference IFN-I signature score and wherein the TLR inhibitor is a TLR7 and/or TLR8 small molecule inhibitor.

E33. A method for predicting the suitability of an individual with a disease who is receiving treatment with a TLR inhibitor to continue said treatment comprising determining an IFN-I signature score in a sample from the individual, wherein the individual is assessed to be suitable to continue the treatment if the determined IFN-I signature score is higher than a reference IFN-I signature score and unsuitable to continue the treatment if the determined IFN-I signature score is lower than the reference IFN-I signature score and wherein the TLR inhibitor is a TLR7 and/or TLR8 inhibitor of formula I-a,

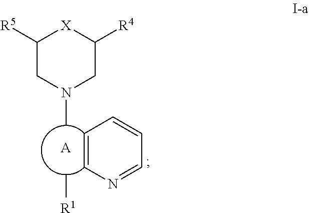

or a pharmaceutically acceptable salt thereof, wherein R¹ is —OMe or —CN, X is O or CH₂, R⁴ is

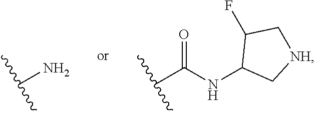

R⁵ is methyl or —CF₃ and Ring A is

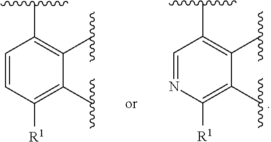

E34. A TLR inhibitor for use in a method of treating a disease in an individual comprising administering the TLR inhibitor to the individual, wherein treatment is based upon IFN-I acitivity in a sample from the individual.

E35. A TLR inhibitor for use in a method of treating a disease in an individual comprising administering the TLR inhibitor to the individual, wherein treatment is based upon IFN-I acitivity in a sample from the individual and wherein the TLR inhibitor is a TLR7 and/or TLR8 small molecule inhibitor.

E36. A TLR inhibitor for use in a method of treating a disease in an individual comprising administering the TLR inhibitor to the individual, wherein treatment is based upon IFN-I acitivity in a sample from the individual and wherein the TLR inhibitor is a TLR7 and/or TLR8 inhibitor of formula I-a,

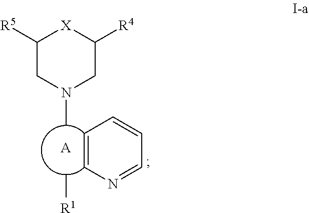

or a pharmaceutically acceptable salt thereof, wherein R¹ is —OMe or —CN, X is O or CH₂, R⁴ is

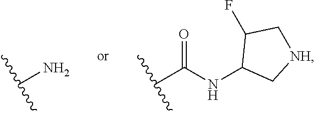

R⁵ is methyl or —CF₃ and Ring A is

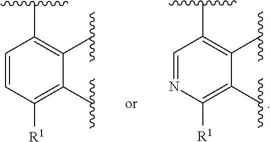

E37. The TLR inhibitor for use according to any one of E34 to E36, wherein the IFN-I activity in a sample from the individual is compared to a reference IFN-I activity and the individual is administered the TLR inhibitor if the IFN-I activity of the individual is higher than the reference IFN-I activity.

E38. A TLR inhibitor for use in a method of treating a disease in an individual comprising selecting an individual having the disease and high IFN-I activity and administering the TLR inhibitor to the individual.

E39. A TLR inhibitor for use in a method of treating a disease in an individual comprising selecting an individual having the disease and high IFN-I activity and administering the TLR inhibitor to the individual, wherein the TLR inhibitor is a TLR7 and/or TLR8 small molecule inhibitor.

E40. A TLR inhibitor for use in a method of treating a disease in an individual comprising selecting an individual having the disease and high IFN-I activity and administering the TLR inhibitor to the individual, wherein the TLR inhibitor is a TLR7 and/or TLR8 inhibitor of formula I-a,

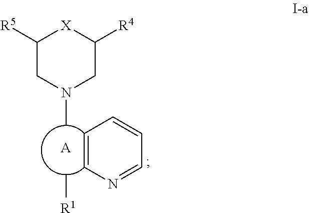

or a pharmaceutically acceptable salt thereof, wherein R¹ is —OMe or —CN, X is O or CH₂, R⁴ is

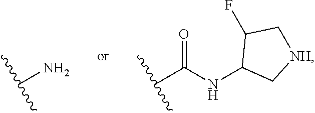

R⁵ is methyl or —CF₃ and Ring A is

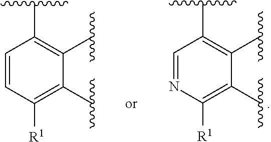

E41. The TLR inhibitor for use according to any one of E38 to E40, wherein the individual is selected as having high IFN-I activity by comparing the IFN-I activity in a sample from the individual to a reference IFN-I activity and determining that the IFN-I activity of the individual is above the reference IFN-I activity.

E42. A TLR inhibitor for use in a method of treating a disease in an individual comprising administering the TLR inhibitor to the individual, wherein treatment is based upon an IFN-I signature expression pattern in a sample from the individual.

E43. A TLR inhibitor for use in a method of treating a disease in an individual comprising administering the TLR inhibitor to the individual, wherein treatment is based upon an IFN-I signature expression pattern in a sample from the individual, wherein the TLR inhibitor is a TLR7 and/or TLR8 small molecule inhibitor.

E44. A TLR inhibitor for use in a method of treating a disease in an individual comprising administering the TLR inhibitor to the individual, wherein treatment is based upon an IFN-I signature expression pattern in a sample from the individual, wherein the TLR inhibitor is a TLR7 and/or TLR8 inhibitor of formula I-a,

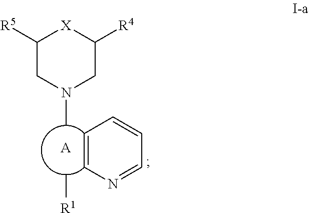

or a pharmaceutically acceptable salt thereof, wherein R¹ is —OMe or —CN, X is O or CH₂, R⁴ is

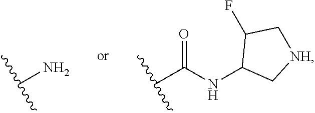

R⁵ is methyl or —CF₃ and Ring A is

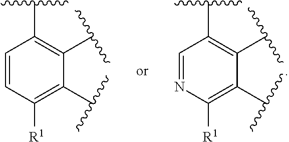

E45. A TLR inhibitor for use in a method of treating a disease in an individual comprising determining an IFN-I signature score in a sample from the individual and administering the TLR inhibitor to the individual if the IFN-I signature score is higher than a reference IFN-I signature score.

E46. A TLR inhibitor for use in a method of treating a disease in an individual comprising determining an IFN-I signature score in a sample from the individual and administering the TLR inhibitor to the individual if the IFN-I signature score is higher than a reference IFN-I signature score, wherein the TLR inhibitor is a TLR7 and/or TLR8 small molecule inhibitor.

E47. A TLR inhibitor for use in a method of treating a disease in an individual comprising determining an IFN-I signature score in a sample from the individual and administering the TLR inhibitor to the individual if the IFN-I signature score is higher than a reference IFN-I signature score, wherein the TLR inhibitor is a TLR7 and/or TLR8 inhibitor of formula I-a,

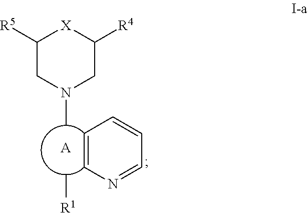

or a pharmaceutically acceptable salt thereof, wherein R¹ is —OMe or —CN, X is O or CH₂, R⁴ is

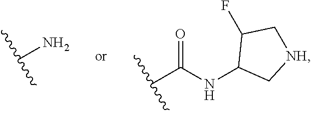

R⁵ is methyl or —CF₃ and Ring A is

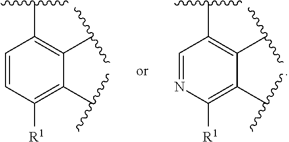

E48. A combination of a TLR inhibitor and a corticosteroid for use in a method of treating a disease in an individual comprising selecting an individual having the disease and high IFN-I activity and administering the combination to the individual.

E49. A combination of a TLR inhibitor and a corticosteroid for use in a method of treating a disease in an individual comprising selecting an individual having the disease and high IFN-I activity and administering the combination to the individual, wherein the TLR inhibitor is a TLR7 and/or TLR8 small molecule inhibitor and the corticosteroid is a glucocorticosteroid.

E50. A combination of a TLR inhibitor and a corticosteroid for use in a method of treating a disease in an individual comprising selecting an individual having the disease and high IFN-I activity and administering the combination to the individual, wherein the TLR inhibitor is a TLR7 and/or TLR8 inhibitor of formula I-a,

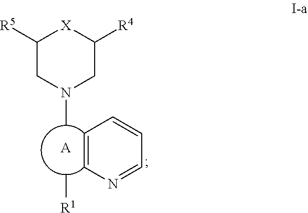

or a pharmaceutically acceptable salt thereof, wherein R¹ is —OMe or —CN, X is O or CH₂, R⁴ is

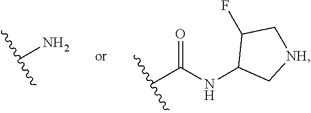

R⁵ is methyl or —CF₃ and Ring A is

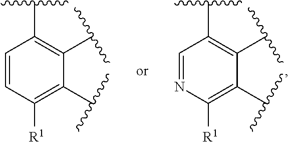

and wherein the corticosteroid is a glucocorticosteroid.

E51. The combination for use according to any one of E48 to E50, wherein the individual is selected as having high IFN-I activity by comparing the IFN-I activity in a sample from the individual to a reference IFN-I activity and determining that the IFN-I activity of the individual is above the reference IFN-I activity.

E52. A combination of a TLR inhibitor and a corticosteroid for use in a method of treating a disease in an individual comprising determining an IFN-I signature score in a sample from the individual and administering the combination to the individual if the IFN-I signature score is higher than a reference IFN-I signature score.

E53. A combination of a TLR inhibitor and a corticosteroid for use in a method of treating a disease in an individual comprising determining an IFN-I signature score in a sample from the individual and administering the combination to the individual if the IFN-I signature score is higher than a reference IFN-I signature score, wherein the TLR inhibitor is a TLR7 and/or TLR8 small molecule inhibitor, and wherein the corticosteroid is a glucocorticosteroid.

E54. A combination of a TLR inhibitor and a corticosteroid for use in a method of treating a disease in an individual comprising determining an IFN-I signature score in a sample from the individual and administering the TLR inhibitor to the individual if the IFN-I signature score is higher than a reference IFN-I signature score, wherein the TLR inhibitor is a TLR7 and/or TLR8 inhibitor of formula I-a,

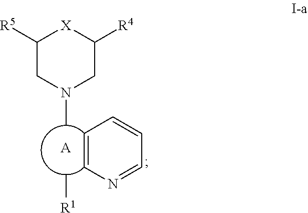

or a pharmaceutically acceptable salt thereof, wherein R¹ is —OMe or —CN, X is O or CH₂, R⁴ is

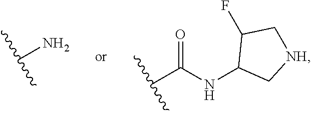

R⁵ is methyl or —CF₃ and Ring A is

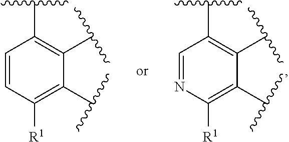

and wherein the corticosteroid is a glucocorticosteroid.

E55. The method, TLR inhibitor or combination for use according to any one of E9, E10, E11, E20, E21, E22, E31, E32, E33, E45, E46, E47, E52, E53 and E54, wherein the determination of the IFN-I signature score comprises the following steps:
(i) obtaining a sample from the individual;
(ii) measuring the expression level for each gene in the IFN-I signature in the sample;
(iii) normalizing each of the gene expression levels; and
(iv) calculating the arithmetic mean of the normalized gene expression levels to obtain the IFN-I signature score.

E56. The method or TLR inhibitor for use according to any one of E6, E7, E8, E17, E18, E19, E28, E29, E30, E42, E43 and E44, wherein the IFN-I signature of the IFN-I signature expression pattern comprises one or more genes selected from the group consisting of BST2, CMPK2, CXCL10, EPSTII, GBP5, HERC5, HERC6, IFI6, IFI27, IFI44, IFI44L, IFIH1, IFIT1, IFIT2, IFIT3, IRF7, ISG15, LY6E, MX1, MX2, OAS1, OAS2, OAS3, OASL, PKR, RSAD2, SIGLEC1, STAT1, TNFSF10 and USP18.

E57. The method or TLR inhibitor for use according to E56, wherein the IFN-I signature of the IFN-I signature expression pattern comprises one or more genes selected from the group consisting of HERC5, IFI27, IFIT1 and RSAD2.

E58. The method or TLR inhibitor for use according to E57, wherein the IFN-I signature of the IFN-I signature expression pattern comprises or consists of HERC5, IFI27, IFIT1 and RSAD2.

E59. The method, TLR inhibitor or combination for use according to any one of E9, E10, E11, E20, E21, E22, E31, E32, E33, E45, E46, E47 and E55, wherein the IFN-I signature of the IFN-I signature score comprises one or more genes selected from the group consisting of BST2, CMPK2, CXCL10, EPSTI1, GBP5, HERC5, HERC6, IFI6, IFI27, IFI44, IFI44L, IFIH1, IFIT1, IFIT2, IFIT3, IRF7, ISG15, LY6E, MX1, MX2, OAS1, OAS2, OAS3, OASL, PKR, RSAD2, SIGLEC1, STAT1, TNFSF10 and USP18.

E60. The method, TLR inhibitor or combination for use according to E59, wherein the IFN-I signature of the IFN-I signature score comprises one or more genes selected from the group consisting of HERC5, IFI27, IFIT1 and RSAD2.

E61. The method, TLR inhibitor or combination for use according to E60, wherein the IFN-I signature of the IFN-I signature score comprises or consists of HERC5, IFI27, IFIT1 and RSAD2.

E62. The method, TLR inhibitor or combination for use according to any one of E9, E10, E11, E20, E21, E22, E31, E32, E33, E45, E46, E47 and E55, wherein the IFN-I signature of the IFN-I signature score consists of the genes HERC5, IFI27, IFIT1 and RSAD2 and the reference IFN-I signature score is -0.5.

E63. The method, TLR inhibitor or combination for use according to E55, wherein the IFN-I signature of the IFN-I signature score consists of the genes HERC5, IFI27, IFIT1 and RSAD2, the genes ACTB, GAPDH and TFRC are used for normalization and the reference IFN-I signature score is -0.5.

E64. The method, TLR inhibitor or combination for use according to any one of E5, E16, E27, E37, E41 and E51, wherein the reference IFN-I activity is defined as follows:
(i) Defining a population of patients having the same disease as the individual whose IFN-I activity is determined and who are treated with the same TLR inhibitor as said individual;
(ii) Determining the IFN-I activity in a sample from each patient in the patient population before treatment with the TLR inhibitor;
(iii) Determining therapeutic effectiveness for each patient in the patient population after treatment with the TLR inhibitor;
(iv) Dividing the patient population into a group that shows more therapeutic effectiveness and a group that shows less therapeutic effectiveness; and
(v) Defining the IFN-I activity that divides the two patient groups as the reference IFN-I activity.

E65. The method, TLR inhibitor or combination for use according to any one of E9, E10, E11, E20, E21, E22, E31, E32, E33, E45, E46, E47, E52, E53 and E54, wherein the reference IFN-I signature score is defined as follows:
(i) Defining a population of patients having the same disease as the individual whose IFN-I signature score is determined and who are treated with the same TLR inhibitor as said individual;
(ii) Determining the IFN-I signature score in a sample from each patient in the patient population before treatment with the TLR inhibitor, wherein the IFN-I signature score is based on the same IFN-I signature as the IFN-I signature on which the IFN-I signature score of the individual whose IFN-I signature score is determined is based on;
(iii) Determining therapeutic effectiveness for each patient in the patient population after treatment with the TLR inhibitor;
(iv) Dividing the patient population into a group that shows more therapeutic effectiveness and a group that shows less therapeutic effectiveness; and
(v) Defining the IFN-I signature score that divides the two patient groups as the reference IFN-I signature score.

E66. The method, TLR inhibitor or combination for use according to any one of E1 to E65, wherein the disease is caused, mediated and/or propagated by TLR activity.

E67. The method, TLR inhibitor or combination for use according to any one of E1 to E66, wherein the disease is an autoimmune disease or a viral disease.

E68. The method, TLR inhibitor or combination for use according to any one of E1 to E67, wherein the disease is selected from the group consisting of rheumatoid arthritis, systemic lupus erythematosus, cutaneous lupus erythematosus, lupus nephritis, type I diabetes mellitus, multiple sclerosis, Sjogren's disease, polymyositis and dermatomyositis.

E69. The method, TLR inhibitor or combination for use according to any one of E1 to E66, wherein the disease is COVID-19.

E70. The method, TLR inhibitor or combination for use according to any one of E1 to E66, wherein the disease is systemic lupus erythematosus or cutaneous lupus erythematosus.

E71. The method, TLR inhibitor or combination for use according to any one of E1 to E66, wherein the disease is polymyositis or dermatomyositis.

E72. The method, TLR inhibitor or combination for use according to any one of E1 to E71, wherein the TLR inhibitor is selected from the group consisting of:

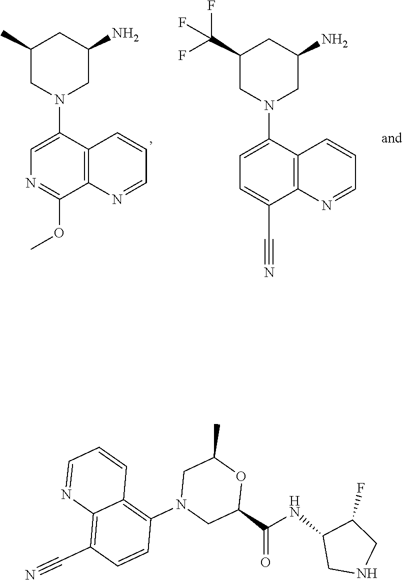 [SMILES: COc1ncc(N2C[C@@H](C)C[C@@H](N)C2)c2cccnc12 ; N#Cc1ccc(N2C[C@H](N)C[C@H](C(F)(F)F)C2)c2cccnc12 ; C[C@@H]1CN(c2ccc(C#N)c3ncccc23)C[C@H](C(=O)N[C@H]2CNC[C@H]2F)O1]

or a pharmaceutically acceptable salt thereof.

E73. The method, TLR inhibitor or combination for use according to E72, wherein the TLR inhibitor is

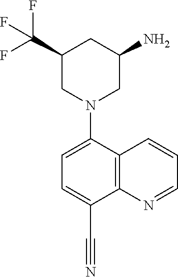 [SMILES: N#Cc1ccc(N2C[C@H](N)C[C@H](C(F)(F)F)C2)c2cccnc12]

or a pharmaceutically acceptable salt thereof.

All the references cited herein are incorporated by reference in the disclosure of the invention hereby.

Although methods and materials similar or equivalent to those described herein can be used in the practice or testing of the present invention, suitable examples are described below. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXEMPLIFICATION

### Example 1: Comparison of IFN-I signatures

The purpose of this experiment was to evaluate the correlation of the Dx 4 IFN-I signature to the various published IFN-I signature of Table 1:

TABLE 1
IFN-I signatures used for correlation analysis:

| IFN-I | | |
|---|---|---|
| signature | Genes | Reference |
| Dx_4 | HERC5, IFI27, IFIT1, RSAD2 | |
| Anif_5 | IFI27, IFI44, IFI44L, IFI6, | Guo et al., J Invest |
| | RSAD2 | Dermatol. 2015 October; |
| | | 135(10): 2402-2409 |
| Ront_3 | HERC5, EPSTI1, CMPK2 | Kalunian et al., Ann Rheum |
| | | Dis. 2016 January; 75(1): |
| | | 196-202 |
| Ront_7 | IFI27, IFI44, MX1, IFIT1, | McBride et al., Arthritis |
| | OAS1, OAS2, OAS3 | Rheum. 2012 November; |
| | | 64(11): 3666-76 |
| Sifa_4 | IFI27, IFI44, IFI44L, RSAD2 | Khamashta et al., Ann |
| | | Rheum Dis. 2016 |
| | | November; 75(11): |
| | | 1909-1916 |
| EMD_9 | CMPK2, CXCL10, EPSTI1, | |
| | HERC5, IFI27, IFI44, IFI44L, | |
| | IFI6, RSAD2 | |

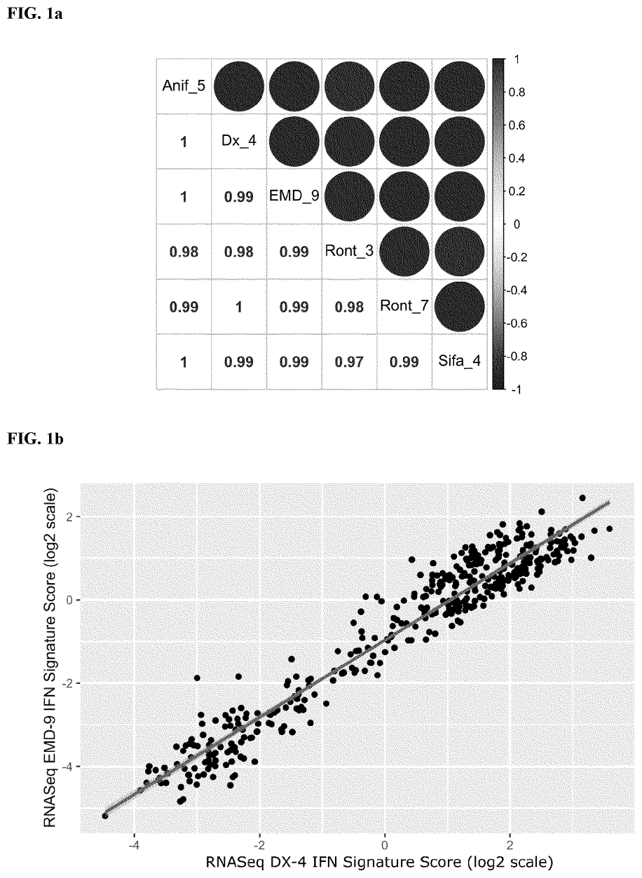

To compare the IFN-I signatures, gene expression data from 417 placebo-treated SLE patients from clinical study NCT01972568 was used. For each SLE patient, an IFN-I signature score was calculated for each of the IFN-I signatures of Table 1 and the correlation coefficient r was calculated (the closer r is to zero, the weaker the linear relationship and the values 1 and 1 represent a perfect positive and negative correlation, respectively). As reflected by FIG. 1, all of the IFN-I signatures, including the DX_4 IFN-I signature, were found to be highly correlated. Therefore, the DX_4 IFN-I signature represents a comparable measure of Type I IFN status to other IFN-I signatures previously reported in the literature.

### Example 2: Use of IFN-I Signatures as a Biomarker

An exploratory phase II clinical study in patients hospitalized with COVID-19 pneumonia was conducted to inter alia assess whether the TLR inhibitor enpatoran improves time to recovery (NCT04448756).

Statistical testing was considered exploratory, and results are presented with no adjustment of type I error for multiplicity. Time to recovery was defined as time from Day 1 to first occurrence of WHO 9-point ordinal scale of ≤3 and was estimated via Kaplan-Meier (KM) analysis, presented with two-sided 95% confidence intervals (CI). The effect of each dose level compared with placebo was evaluated using a stratified log-rank test.

Blood was collected directly in PAXgene RNA tubes from patients at baseline prior to treatment for measurement of gene expression. IFN-I activity was measured by DxTerity Diagnostics (Rancho Dominguez, California, USA), using the IFN-1 test, a commercially available chemical ligation-dependent probe amplification and gene expression test with relative quantitative analysis by capillary electrophoresis. Sample testing and analysis was performed as previously described (Kim et al., J Mol Diagn. 2015 March;17 (2): 118-27). The IFN-1 test measures the expression levels of four IFN response genes (HERC5, IFI27, IFIT1 and RSAD2), i.e. the Dx-4 IFN-I signature, relative to the expression levels of three housekeeping normalizer genes (ACTB, GAPDH and TFRC). Normalized expression values of each respective response gene were calculated per the following function: Normalized Expression_{Gene i}=Log₂(Height_{Gene i})−Mean (Log₂ (Normaliser Gene Height)). The IFN-I signature score was calculated by averaging the normalized expression values of the four response genes. The cut-off between high IFN-I signature and low IFN-I signature scores (−0.5), was set to the mean±2 SDs (95^{th} percentile) of IFN-I signature scores from 281 healthy human donors. This cut-off falls within the trough of the observed bimodal distribution of IFN-I signature scores for cohorts of SLE patients.

149 patients received either placebo (n=49), or enpatoran 50 mg (n=54)₀ᵣ 100 mg (n=46) BID. Mean IFN-I signature scores (50 mg BID-0.84; 100 mg BID-0.74; placebo-0.91) and the proportion of patients with high IFN-I signature scores at baseline (50 mg BID 35%; 100 mg BID 43%; placebo 35%) were reasonably consistent across treatment groups, as reflected by Table 2:

TABLE 2
Baseline IFN-signature characteristics:

| | All Subjects | Placebo | Enp 50 BID | Enp 100 BID |
|---|---|---|---|---|
| N | 134 | 46 | 46 | 42 |
| IFN-I signature | [−4.08, 3.47] | [−4.08, 2.70] | [−3.45, 3.47] | [−3.32, 2.64] |
| scores range | | | | |
| IFN-I signature | −0.83 ± 1.72 | −0.91 ± 1.74 | −0.84 ± 1.65 | −0.74 ± 1.82 |
| scores Mean ± | | | | |
| SD | | | | |
| IFN-I signature | −1.11 | −1.08 | −1.02 | −1.42 |
| scores Median | | | | |
| High IFN-I | 50 (37%) | 16 (35%) | 16 (35%) | 18 (43%) |
| signature | | | | |
| scores N (%) | | | | |
| Low IFN-I | 84 (63%) | 30 (65%) | 30 (65%) | 24 (57%) |
| signature | | | | |
| scores N (%) | | | | |

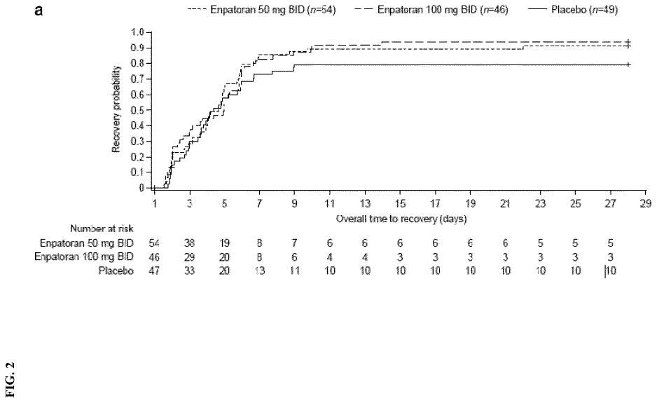

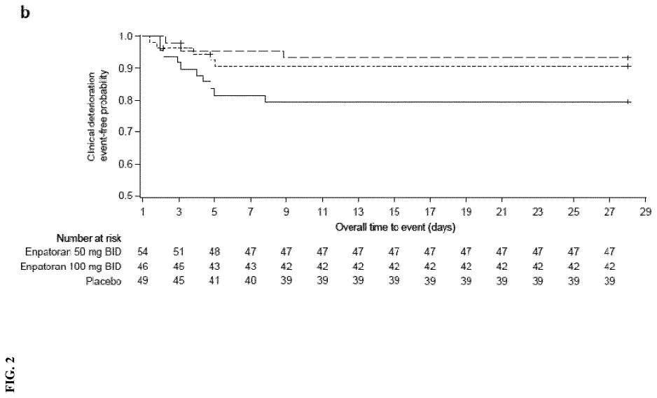

The primary efficacy endpoint of the time to recovery from Day 1 through Day 28 was not met for the unstratified patient populations, despite a numerical trend towards higher recovery rates in both enpatoran groups (50 mg BID n=48, 88.9%, P=0.054; 100 mg BID n=42, 91.3%, P=0.107) compared with the placebo group (n=37, 75.5%). The median time to recovery was similar across groups (3.4-3.9 days), with a lack of differentiation until Day 6 (FIG. 2).

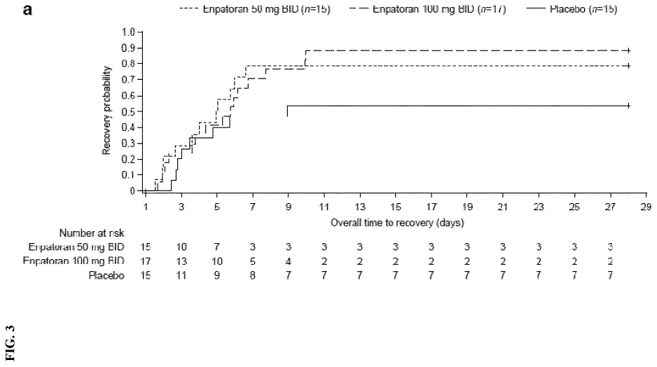

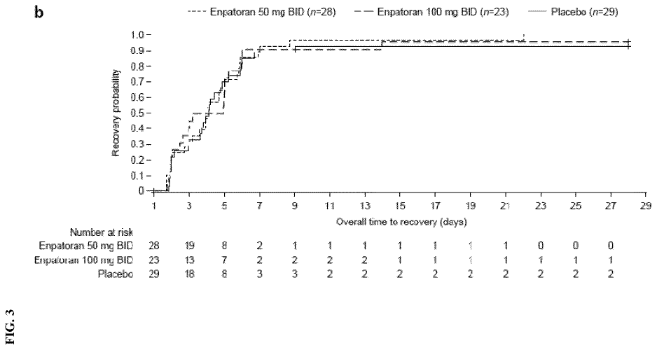

In the subgroup with high IFN-I signature scores at baseline, the KM-estimated cumulative recovery rates up to Day 14 were higher for patients who received enpatoran (50 mg BID n=11, 78.6% [95% CI 55.2, 94.8]; 100 mg BID n=15, 88.2% [68.8, 98.0]) compared with those who received placebo (n=8, 53.3% [31.1, 78.8]; FIG. 3). This was consistent to Day 28, when recovery rates were 73.3% (P=0.031) and 88.2% (P=0.031) with enpatoran 50 mg BID and enpatoran 100 mg BID, respectively, and 53.3% with placebo. Placebo-treated patients in the subgroup with low IFN-I signature scores at baseline had higher recovery rates at Day 28 (n=25, 86.2%) than those in the high IFN-I signature subgroup, and there was a lack of differentiation between placebo and enpatoran (50 mg BID n=28, 100%, P=0.236; 100 mg BID n=21, 91.3%, P=0.458).

These exploratory analyses suggest that the time to recovery was improved with enpatoran versus placebo for patients with broad immune activation determined by high IFN-I signature scores at baseline and, more generally, suggest IFN-I activity as a potential predictive biomarker for TLR inhibitors, such as enpatoran, in COVID-19 patients, as well as other autoimmune and inflammatory diseases, such as lupus.

### Example 3: Co-Treatment with a TLR Inhibitor and Corticosteroids in the Context of IFN-α Pre-Treatment

Previous studies showed that IFN-α can reduce the potency of GCs (Guiducci et al., Nature. 2010 Jun. 17;465 (7300): 937-41). Thus, the purpose of this experiment was to determine if pre-treatment with IFN-α impacts the response of peripheral blood mononuclear cells (PBMCs) to TLR7 and/or TLR8 agonists and if potency of the glucocorticosteroid dexamethasone (Dex) is reduced further.

### Materials and Methods

Blood was obtained from healthy donor leukopaks (New York Blood Center, New York, USA). PBMCs were isolated using ACCUPSIN Tubes according to the manufacturer's protocol (Sigma-Aldrich, Missouri, USA). Cell viability was assessed with Trypan Blue Stain (BioRad, California, USA). Cells were cultured in RPMI 1640 Medium (Gibco, ThermoFisher Scientific, Massachusetts, USA) with 10% Fetal Bovine Serum (Corning, Arizona, USA) and 1× Penicillin-Streptomycin (Gibco).

PBMCs were pre-treated for 15 minutes with Dex (Sigma-Aldrich) starting at 10 μM with a 3-fold serial dilution and 1 μM CMPD2 (TLR7 and TLR8 inhibitor synthesized in-house; structure published previously (Vlach et al., J Pharmacol Exp Ther. 2021 March;376 (3): 397-409)) with a 2-fold serial dilution. Cells were then stimulated with 3-5 μM of the TLR7 and 8 agonist R848 (InvivoGen, Toulouse, France), 3 μM of the TLR7 agonist CL-087 (synthesized in-house), or 1 μM of the TLR8 agonist motolimod (Selleckchem, Texas, USA). Plates were incubated overnight at 37° C. and 5% CO₂. Cell viability was assessed using CellTiter Glo Luminescent Cell Viability Assay (Promega, Wisconsin, USA).

Cytokine secretion in supernatants was detected using AlphaLISA Detection Kits for human interleukin-6 (IL-6), tumor necrosis factor -alpha (TNF-α) and IFN-alpha (IFN-α) following the manufacturer's protocol (Perkin Elmer, Ohio, USA). When pre-treatment with cytokines was required, PBMCs were exposed to 10 ng/mL IFN-α2a (Sigma-Aldrich) for 4-5 hours before being treated with the compounds and TLR7 and/or 8 agonists described above.

Data were analyzed using Graphpad Prism (Dotmatics, Massachusetts, USA). Group medians were determined and statistical significance was tested using a Kruskal-Wallis test, ANOVA or T test (as described in the figure legends). Synergistic effects were evaluated on Loewe matrix plots using Combenefit (SourceForge, California, USA), as described by (Di Veroli et al., Bioinformatics. 2016 Sep. 15;32 (18): 2866-8), and synergy scores were calculated from the area under the curve.

### Results

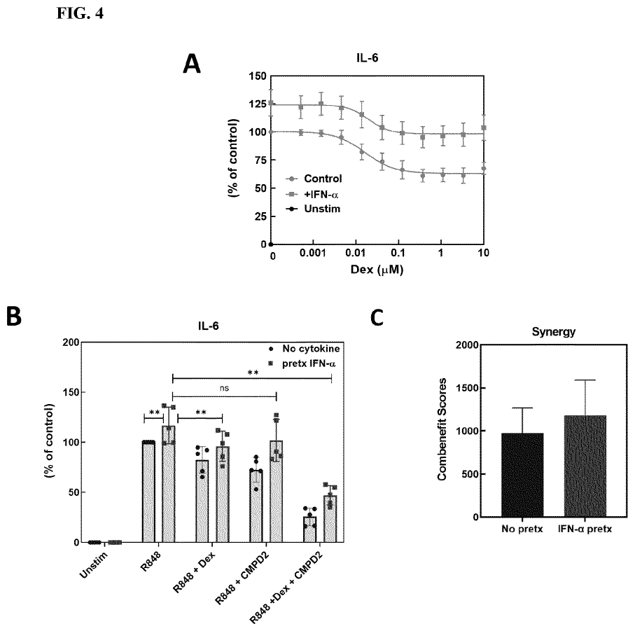

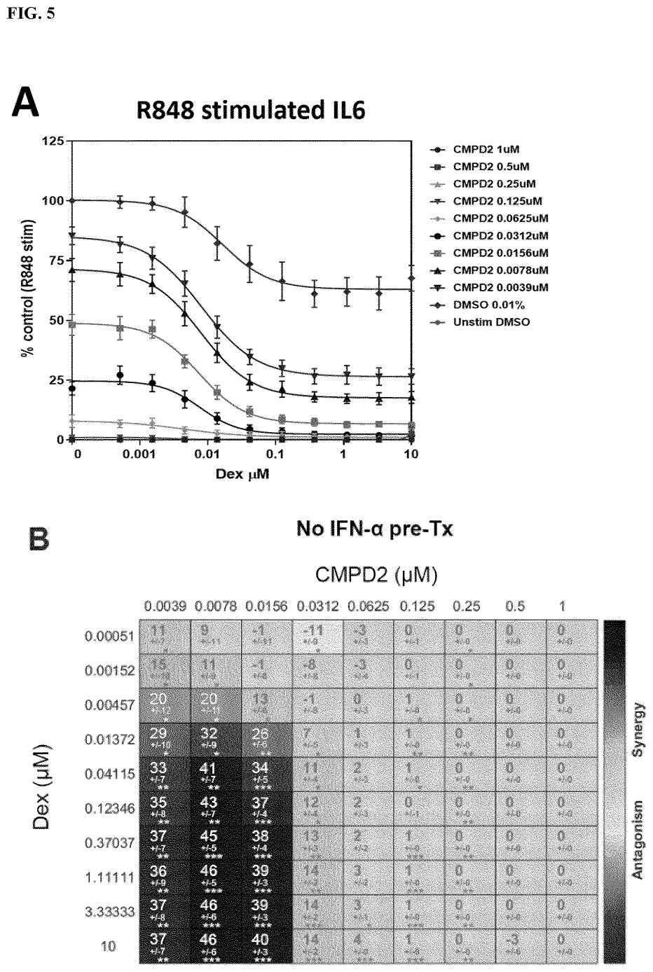

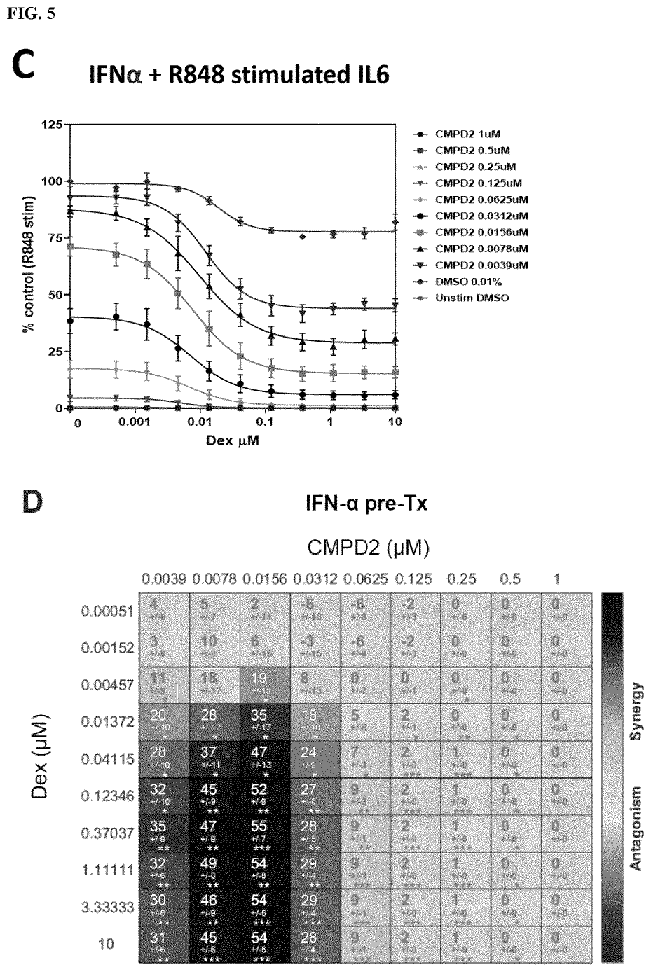

It was found that IFN-α pre-treatment increased the responsiveness of PBMCs to R⁸⁴⁸, as measured by IL-6 secretion (FIGS. 4A and B; FIGS. 5A and C). Dex alone was less potent at blocking R⁸⁴⁸-induced IL-6 secretion in IFN-α pre-treated cells than Dex in combination with CMPD2. The low dose Dex and CMPD2 combination treatment reduced this heightened responsiveness (FIG. 4B) and synergy was confirmed by Combenefit analysis (FIGS. 5B and D), with similar synergy scores in R⁸⁴⁸-stimulated cells with and without IFN-α pre-treatment (FIG. 4C). These results were not due to changes in cell viability, which did not change greatly across the matrix titration doses tested (data not shown).

In summary, these results suggest that even in the context of a pre-existing pro-inflammatory environment, which can be envisioned in autoimmune diseases such as lupus, the inhibition of TLR7 and/or TLR8 can potentiate the action of glucocorticosteroids to provide synergistic anti-inflammatory benefits.

### Example 4: IFN-α Stimulation by RNA-Containing Immune Complexes

In order to further confirm the link between TLR7 and/or TLR8 and the expression of IFN-I and interferon-stimulated genes (ISGs), immune complexes of patients having autoimmune diseases were tested for their ability to stimulate expression of IFN-α and ISGs in healthy donor peripheral blood mononuclear cells (PBMCs), as well as the ability of the TLR inhibitor enpatoran to block such stimulation.

### Materials and Methods

Blood sample collection: Blood samples were collected from subjects with lupus nephritis (LN), systemic lupus erythematosus (SLE), dermatomyositis (DM), polymyositis (PM), patients with inclusion body myositis (IBM) and healthy controls (HC). Blood plasma was isolated and frozen. Later, immunoglobulin G (IgG) was purified using protein A resin and the protein concentration determined.

Immune complex formation and peripheral blood mononuclear cell (PBMC) stimulation: To generate necrotic cell lysates, human embryonic kidney 293 cells were suspended at a concentration of 50 x10⁶ cells/mL in phosphate buffered saline (PBS; Gibco, Grand Island, NY). The cells were frozen at −80° C. for 10 minutes followed by thawing at 37° C. Four freeze/thaw cycles were performed. The lysate was centrifuged at 400 g for 5 minutes to separate non-lysed cells and the supernatant was collected as necrotic cell lysate.

PBMCs were isolated from leukopacks collected from healthy donors (New York Blood Center, New York, NY) in sodium heparin tubes using Ficoll-Paque Plus (Cytiva Life Sciences, Uppsala, Sweeden). The cells were plated in 96-well U-bottom plates (4 x10⁵ cells/well) in RPMI 1640 Medium (Gibco, Grand Island, NY) supplemented with 10% fetal bovine serum (FBS; Corning, Woodland, CA). Before stimulation, PBMCs were pre-treated with 1 μM of enpatoran for 30 minutes. The necrotic cell lysate (10% vol/vol) and IgG (0.1 mg/mL) purified from patients' plasma were added to the PBMCs, which were incubated at 37° C. for 24 hours. The supernatants were collected and cytokine production was measured by AlphaLISA (PerkinElmer, Waltham, MA).

NanoString analysis: Gene expression in purified RNA samples was analyzed by NanoString. A 46-gene custom panel with markers of inflammation was used. A total of 500 ng of RNA per sample was run on the nCounter Pro Analysis System (NanoString, Seattle, WA). The data were processed using nSolver (NanoString) and Log₂ fold changes were calculated for each sample relative to cells treated with supernatants from control PBMCs.

### Results

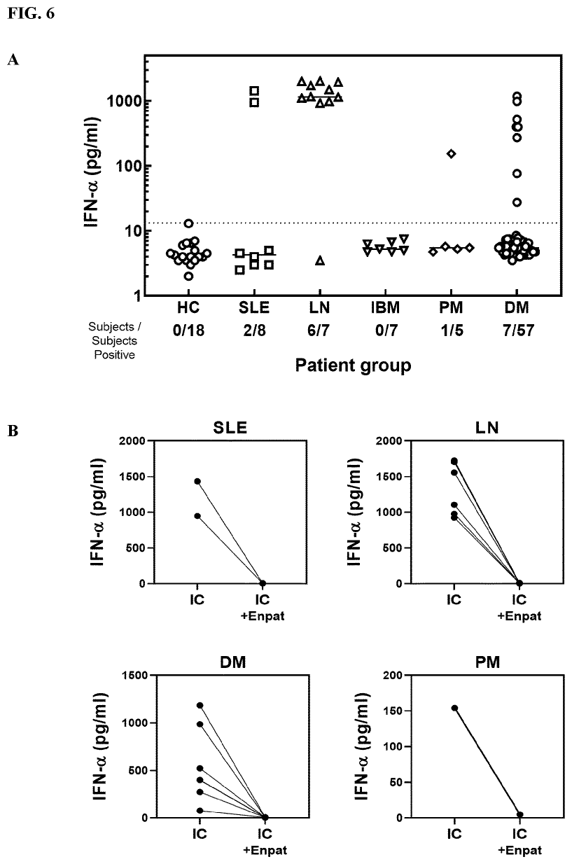

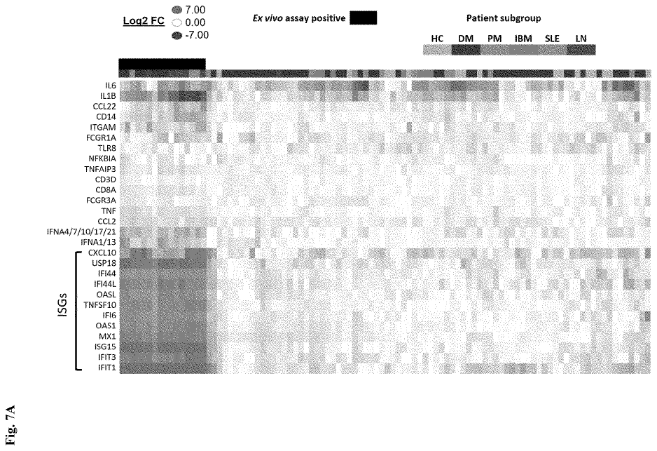

IgG isolated from 69 patients with idiopathic inflammatory myopathy (IIM), including DM, PM and IBM, as well as 15 patients with lupus and 18 healthy controls was combined with necrotic cell lysate to form immune complexes which were then added to healthy donor PBMCs. IFN-α production was stimulated by immune complexes generated using IgG from 6/7 of the LN patients tested and 2/8 of the SLE patients tested (FIG. 6A). In the IIM subsets, immune complexes from patients with PM and DM showed activity for IFN-α, while IBM did not.

When the PBMCs were pre-treated with enpatoran, IFN-α production was completely inhibited (FIG. 6B), demonstrating that IFN-α production was mediated by TLR7 and/or TLR8. IgG and necrotic cell lysate alone had no stimulatory activity (data not shown).

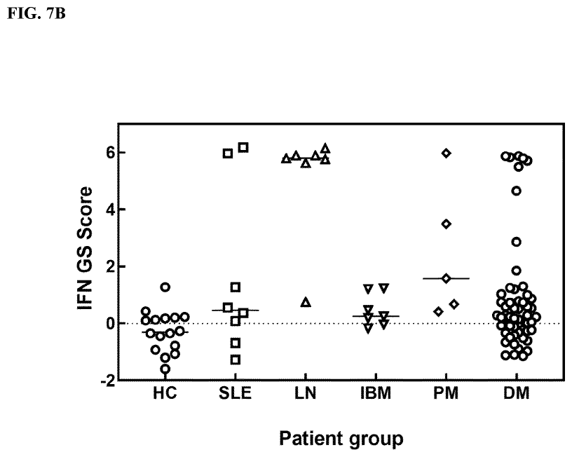

Gene expression analysis on the PBMC lysates showed that the samples which stimulated IFN-α protein also induced changes in gene expression, and the most prominent effect was induction of IFN-stimulated genes (ISGs; FIG. 7A). An IFN-I signature score (IFN GS Score), calculated based on ISG expression, was induced and upregulation was apparent in the patients also positive for protein induction (FIG. 7B).

By demonstrating that the stimulation of IFN-α and ISG expression by immune complexes of autoimmune disease patients is dependent on TLR7 and/or TLR8, it is further supported that IFN-I activity may be suitable as a predictive biomarker for TLR inhibitors.

1. Use of the IFN-I activity as a predictive biomarker for the TLR inhibitor treatment of an individual with a disease;
wherein the TLR inhibitor is a TLR7 and/or TLR8 small molecule inhibitor.

2. A method for predicting therapeutic effectiveness of a TLR inhibitor in an individual with a disease comprising determining the IFN-I activity in a sample from the individual,
wherein the IFN-I activity in the sample indicates the therapeutic effectiveness of the TLR inhibitor; and
wherein the TLR inhibitor is a TLR7 and/or TLR8 small molecule inhibitor.

3. A method for predicting the suitability of an individual with a disease to initiate treatment with a TLR inhibitor comprising determining the IFN-I activity in a sample from the individual,
wherein the IFN-I activity in the sample indicates the suitability of the individual to initiate the treatment; and
wherein the TLR inhibitor is a TLR7 and/or TLR8 small molecule inhibitor.

4. A method for predicting the suitability of an individual with a disease who is receiving treatment with a TLR inhibitor to continue said treatment comprising determining the IFN-I activity in a sample from the individual,
wherein the IFN-I activity in the sample indicates the suitability of the individual to continue the treatment; and
wherein the TLR inhibitor is a TLR7 and/or TLR8 small molecule inhibitor.

5. A TLR inhibitor for use in a method of treating a disease in an individual comprising administering the TLR inhibitor to the individual,
wherein treatment is based upon IFN-I acitivity in a sample from the individual; and
wherein the TLR inhibitor is a TLR7 and/or TLR8 small molecule inhibitor.

6. The method according to claim 2,
wherein the TLR inhibitor is predicted to be therapeutically effective if the IFN-I activity of the individual is determined to be high and predicted not to be therapeutically effective if the IFN-I activity of the individual is determined to be low.

7. The method according to claim 3,
wherein the individual is assessed to be suitable to initiate the treatment if the IFN-I activity of the individual is determined to be high and assessed to be unsuitable to initiate the treatment if the IFN-I activity of the individual is determined to be low.

8. The method according to claim 4,
wherein the individual is assessed to be suitable to continue the treatment if the IFN-I activity of the individual is determined to be high and unsuitable to continue the treatment if the IFN-I activity of the individual is is determined to be low.

9. The TLR inhibitor for use according to claim 5,
wherein the individual is administered the TLR inhibitor if the IFN-I activity of the individual is determined to be high.

10. The method or TLR inhibitor for use according to any one of claims 1 to 9,
wherein the IFN-I activity is determined by determining an IFN-I signature expression pattern or an IFN-I signature score in the sample from the individual.

11. The method according to claim 2,
wherein the IFN-I activity is determined by determining an IFN-I signature score in the sample from the individual; and
wherein the TLR inhibitor is predicted to be therapeutically effective if based on a comparison of the IFN-I signature score in the sample from the individual with a reference IFN-I signature score the individual is determined as having high IFN-I activity and/or wherein the TLR inhibitor is predicted to be therapeutically ineffective if based on a comparison of the IFN-I signature score in the sample from the individual with a reference IFN-I signature score the individual is determined as having low IFN-I activity.

12. The method according to claim 3,
wherein the IFN-I activity is determined by determining an IFN-I signature score in the sample from the individual; and
wherein the individual is assessed to be suitable to initiate the treatment if based on a comparison of the IFN-I signature score in the sample from the individual with a reference IFN-I signature score the individual is determined as having high IFN-I activity and/or wherein the individual is assessed to be unsuitable to initiate the treatment if based on a comparison of the IFN-I signature score in the sample from the individual with a reference IFN-I signature score the individual is determined as having low IFN-I activity.

13. The method according to claim 4,
wherein the IFN-I activity is determined by determining an IFN-I signature score in the sample from the individual; and
wherein the individual is assessed to be suitable to continue the treatment if based on a comparison of the IFN-I signature score in the sample from the individual with a reference IFN-I signature score the individual is determined as having high IFN-I activity and/or wherein the individual is assessed to be unsuitable to continue the treatment if based on a comparison of the IFN-I signature score in the sample from the individual with a reference IFN-I signature score the individual is determined as having low IFN-I activity.

14. The TLR inhibitor for use according to claim 5,
wherein the IFN-I activity is determined by determining an IFN-I signature score in the sample from the individual; and
wherein the TLR inhibitor is administered to the individual if based on a comparison of the IFN-I signature score in the sample from the individual with a reference IFN-I signature score the individual is determined as having high IFN-I activity.

15. The method or TLR inhibitor for use according to any one of claims 1 to 14, wherein the TLR7 and/or TLR8 small molecule inhibitor is selected from the group consisting of 5-[(3R,5S)-3-amino-5-(trifluoromethyl) piperidin-1-yl]quinoline-8-carbonitrile; (3R,5S)-1-(8-methoxy-1,7-naphthyridin-5-yl)-5-methylpiperidin-3-amine; 2-{4-[2-(7,8-dimethyl[1,2,4]triazolo[1,5-a]pyridin-6-yl)-3-(propan-2-yl)-1H-indol-5-yl]piperidin-1-yl}acetamide; rel-(2R,6R)-4-(8-cyanoquinolin-5yl)-N-((3R,4S)-4-fluoropyrrolidin-3-yl)-6-methylmorpholine-2-carboxamide hydrochloride; (S)—N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide; and (R)—N-(4-((5-(1,6-dimethyl-1H-pyrazolo[3,4-b]pyridin-4-yl)-3-methyl-4,5,6,7-tetrahydro-1H-pyrazolo[4,3-c]pyridin-1-yl)methyl)bicyclo[2.2.2]octan-1-yl)morpholine-3-carboxamide or a pharmaceutically acceptable salt of any of these compounds.

16. The method or use according to any one of claims 1 to 14, wherein the TLR7 and/or TLR8 small molecule inhibitor is a compound of formula I-a, 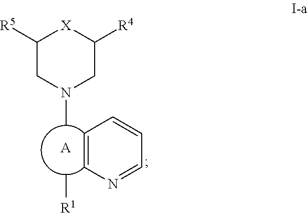
or a pharmaceutically acceptable salt thereof, wherein R¹ is —OMe or —CN, X is O or CH₂, R⁴ is 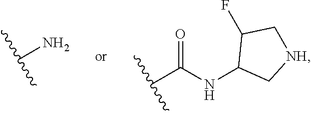
R⁵ is methyl or —CF₃ and Ring A is

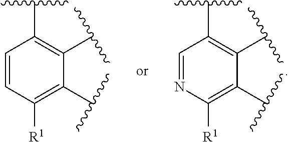

17. The TLR inhibitor for use according to any one of claims 5, 9, 10 and 14, wherein the TLR inhibitor is administered in combination with a glucocorticosteroid.

18. A TLR7 and/or TLR8 inhibitor and a glucocorticosteroid for use in a method of treating a disease in an individual having high IFN-I activity, wherein the method comprises administering the TLR7 and/or TLR8 inhibitor in combination with the glucocorticosteroid to the individual.
